(19) United States
(12) Patent Application Publication
(10) Pub. No.: US 2024/0101626 A1
(43) Pub. Date: Mar. 28, 2024

(54) CONSENSUS PROSTATE ANTIGENS, NUCLEIC ACID MOLECULES ENCODING THE SAME, AND VACCINES AND USES COMPRISING THE SAME

(71) Applicants: Kar MUTHUMANI, Cherry Hill, NJ (US); David WEINER, Merion, PA (US); The Wistar Institute of Anatomy and Biology, Philadelphia, PA (US)

(72) Inventors: Kar Muthumani, Collegeville, PA (US); David Weiner, Merion, PA (US)

(21) Appl. No.: 18/546,306

(22) PCT Filed: Feb. 11, 2022
(86) PCT No.: PCT/US22/16142

§ 371 Date: Aug. 14, 2023

Related U.S. Application Data
(60) Provisional application No. 63/151,229, filed on Feb. 19, 2021.
(60) Provisional application No. 63/148,969, filed on Feb. 12, 2021.

(51) Int. Cl. C07K 14/47; A61K 39/00; A61P 37/04; C12N 9/16

(52) U.S. Cl. CPC C07K 14/4748; A61K 39/001193; A61P 37/04; C12N 9/16; C12Y 301/03002; A61K 39/00; A61K 2039/53

(57) ABSTRACT

The present invention provides consensus amino acid sequences of prostate antigens that are capable of breaking tolerance in a targeted species, including PAP, PARM, PCTA, PSCA, PSP94, and STEAP antigens. Also provided are nucleic acid sequences that encode one or more consensus amino acid sequences of prostate antigens PAP, PARM, PCTA, PSCA, PSP94, and STEAP antigens, as well as genetic constructs/vectors and vaccines expressing the sequences. Also provided herein are methods for generating an autoimmune response against prostate cancer cells by administering one or more of the vaccines, proteins, and/or nucleic acid sequences that are provided.

### CROSS-REFERENCE TO RELATED APPLICATIONS

This application is entitled to priority under 35 U.S.C. § 119(e) to and claims the benefit of U.S. Provisional Application No. 63/148,969, filed Feb. 12, 2021, and U.S. Provisional Application No. 63/151,229, filed Feb. 19, 2021, the disclosures of which are hereby incorporated by reference herein in their entireties.

### BACKGROUND OF THE INVENTION

Prostate cancer (PCa) represents the most prevalent cancer type among males across the globe (Rheinbay E et al., 2020, Nature, 578:102-111; Lucas A R et al., 2020, PLoS One, 15:e0228773). It is the second leading cause of mortality due to cancer among American men and accounts for an estimated 191,930 cases diagnosed in 2020 with 33,330 deaths (Siegel R L et al., 2020, Cancer J Clin, 70:7-30). There have been important advances in management of prostate cancer. Treatment modalities, such as surgery, radiation, chemo, and hormone therapies, have improved outcomes in patients with early-stage PCa. However, recurrence and progression of disease has been reported in 30-40% of patients who undergo radical prostatectomy, as defined by increased prostate serum antigen (PSA) levels in sera. Therapy for advanced stages of this disease presents a major challenge (Yang B et al., 2020, Urol Int, 1-8). This supports advancing novel therapeutic strategies for improving management of PCa. Recent studies suggested that immunotherapy for the induction of immune responses in PCa is among a handful of highly promising therapeutic strategies to be further advanced (Gerritsen W R, 2012, Oncol., 23, Suppl 8:viii22-7; van den Eertwegh A J et al., 2012, Lancet Oncol, 13:509-517; Gerritsen W R et al., 2012, J Clin Immunol., 32:25-35). The determination of suitable antigens with expression confined to relevant tumors and high potential for immunogenicity in human, still remains a challenge (Duperret E K et al., 2018, Cancer Res., 78:6363-6370; Duperret E K et al., 2018, Clin Cancer Res., 24:1190-1201).

Thus, there is a need in the art for nucleic acid constructs that encode prostate cancer antigens and for compositions useful to induce immune responses against prostate cancer antigens and thus break immune tolerance. There is also a need in the art for effective prophylactic and therapeutic vaccines against prostate cancer that are economical and effective.

### SUMMARY OF THE INVENTION

In one aspect, the present invention relates, in part, to a nucleic acid molecule comprising a coding sequence encoding one or more proteins selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof, and/or comprising one or more nucleotide sequences selected from SEQ ID NO:1, a coding sequence that is at least about 90% homologous to SEQ ID NO:1; SEQ ID NO:3, a coding sequence that is at least about 90% homologous to SEQ ID NO:3; SEQ ID NO:5, a coding sequence that is at least about 90% homologous to SEQ ID NO:5; SEQ ID NO:7, a coding sequence that is at least about 90% homologous to SEQ ID NO:7; SEQ ID NO:9, a coding sequence that is at least about 90% homologous to SEQ ID NO:9; SEQ ID NO:11, a coding sequence that is at least about 90% homologous to SEQ ID NO: 11; SEQ ID NO:13, a coding sequence that is at least about 90% homologous to SEQ ID NO: 13; SEQ ID NO:15, a coding sequence that is at least about 90% homologous to SEQ ID NO: 15; SEQ ID NO:17, a coding sequence that is at least about 90% homologous to SEQ ID NO: 17; SEQ ID NO:19, a coding sequence that is at least about 90% homologous to SEQ ID NO: 19; SEQ ID NO:21, a coding sequence that is at least about 90% homologous to SEQ ID NO:21; and/or SEQ ID NO:23, a coding sequence that is at least about 90% homologous to SEQ ID NO:23.

In some embodiments, the nucleic acid molecule comprises a coding sequence encoding one or more proteins selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments, the nucleic acid molecule comprises nucleic acid molecule comprising one or more nucleotide sequences selected from SEQ ID NO:1, a coding sequence that is at least about 90% homologous to SEQ ID NO:1; SEQ ID NO:3, a coding sequence that is at least about 90% homologous to SEQ ID NO:3; SEQ ID NO:5, a coding sequence that is at least about 90% homologous to SEQ ID NO:5; SEQ ID NO:7, a coding sequence that is at least about 90% homologous to SEQ ID NO:7; SEQ ID NO:9, a coding sequence that is at least about 90% homologous to SEQ ID NO:9; SEQ ID NO:11, a coding sequence that is at least about 90% homologous to SEQ ID NO:11; SEQ ID NO: 13, a coding sequence that is at least about 90% homologous to SEQ ID NO:13; SEQ ID NO:15, a coding sequence that is at least about 90% homologous to SEQ ID NO:15; SEQ ID NO: 17, a coding sequence that is at least about 90% homologous to SEQ ID NO:17; SEQ ID NO: 19, a coding sequence that is at least about 90% homologous to SEQ ID NO:19; SEQ ID NO:21, a coding sequence that is at least about 90% homologous to SEQ ID NO:21; and/or SEQ ID NO:23, a coding sequence that is at least about 90% homologous to SEQ ID NO:23.

In some embodiments, the nucleic acid molecule comprises one or more nucleotide sequences selected from at least one selected from either SEQ ID NO:1, a coding sequence that is at least about 90% homologous to SEQ ID NO:1; or SEQ ID NO:3, a coding sequence that is at least about 90% homologous to SEQ ID NO:3; at least one selected from either SEQ ID NO:5, a coding sequence that is at least about 90% homologous to SEQ ID NO:5; or SEQ ID NO:7, a coding sequence that is at least about 90% homologous to SEQ ID NO:7; at least one selected from either SEQ ID NO:9, a coding sequence that is at least about 90% homologous to SEQ ID NO:9; or SEQ ID NO:11, a coding sequence that is at least about 90% homologous to SEQ ID NO:11; at least one selected from either SEQ ID NO:13, a coding sequence that is at least about 90% homologous to SEQ ID NO:13; or SEQ ID NO:15, a coding sequence that is at least about 90% homologous to SEQ ID NO: 15; at least one selected from either SEQ ID NO:17, a coding sequence that is at least about 90% homologous to SEQ ID NO:17; or SEQ ID NO:19, a coding sequence that is at least about 90% homologous to SEQ ID NO:19; and/or at least one selected from either SEQ ID NO:21, a coding sequence that is at least about 90% homologous to SEQ ID NO:21; and/or SEQ ID NO:23, a coding sequence that is at least about 90% homologous to SEQ ID NO:23.

In some embodiments, the nucleic acid molecule comprises one or more nucleotide sequences selected from SEQ ID NO:1; SEQ ID NO:3; SEQ ID NO:5; SEQ ID NO:7; SEQ ID NO:9; SEQ ID NO:11; SEQ ID NO:13; SEQ ID NO: 15; SEQ ID NO: 17; SEQ ID NO: 19; SEQ ID NO:21; and/or SEQ ID NO:23.

In some embodiments, the nucleic acid molecule encodes one or more proteins selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments, the nucleic acid molecule encodes one or more proteins selected from at least one selected from either selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, or SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, at least one selected from either SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, or SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, at least one selected from either SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, or SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, at least one selected from either SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, or SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof; at least one selected from either SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, or SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, and/or at least one selected from either SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments, the nucleic acid molecule encodes one or more proteins selected from SEQ ID NO:2; SEQ ID NO:4; SEQ ID NO:6; SEQ ID NO:8; SEQ ID NO:10; SEQ ID NO:12; SEQ ID NO:14; SEQ ID NO: 16; SEQ ID NO: 18; SEQ ID NO:20; SEQ ID NO:22; and/or SEQ ID NO:24.

In some embodiments, the sequences encoding said one more proteins are operable linked to regulatory elements.

In some embodiments, the nucleic acid molecule is an expression vector.

In some embodiments, the nucleic acid molecule is an expression vector and sequences encoding said one more proteins are operable linked to regulatory elements.

In some embodiments, the nucleic acid molecule is a plasmid.

In one aspect, the present invention also relates, in part, to a composition comprising at least one nucleic acid molecule of the present invention.

In one aspect, the present invention also relates, in part, to a protein comprising one or more proteins selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments, the protein comprises one or more proteins selected from at least one selected from either selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, or SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, at least one selected from either SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, or SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof; at least one selected from either SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, or SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof; at least one selected from either SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, or SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, at least one selected from either SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, or SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, and/or at least one selected from either SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments, the protein comprises one or more proteins selected from SEQ ID NO:2; SEQ ID NO:4; SEQ ID NO:6; SEQ ID NO:8; SEQ ID NO:10; SEQ ID NO:12; SEQ ID NO: 14; SEQ ID NO: 16; SEQ ID NO: 18; SEQ ID NO:20; SEQ ID NO:22; and/or SEQ ID NO: 24.

In some embodiments, the protein is selected from SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and/or SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In one aspect, the present invention also relates, in part, to a composition comprising at least one protein of the present invention.

In one aspect, the present invention also relates, in part, to a method of treating a subject who has been diagnosed with prostate cancer.

In one aspect, the present invention also relates, in part, to a method of inducing an immune response in a subject.

In some embodiments, the method comprises administering at least one nucleic acid molecule of the present invention to the subject. In some embodiments, the method comprises administering at least one protein of the present invention to the subject. In some embodiments, the method comprises administering at least one composition of the present invention to the subject.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A and FIG. 1B, depicts representative design, generation, characterization, and expression analysis of prostate cancer antigens (PCaA) SEV constructs. FIG. 1A depicts a schematic representation of the PCaA-SEV construct generated.

FIG. 1B depicts representative results of a western blot analysis of SEV of STEAP-1, PAP, PARM1, PCTA, PSCA and PSP94. Human 293T cells were transfected with 2 μg of each DNA vaccines or pMV101 and the cell lysates were collected after 48 hours. 25 μg of each lysate was then separated using polyacrylamide gel electrophoresis and subsequently transblotted; followed by the incubation with respective primary and then secondary antibodies. Cell lysates transfected with different PCaA-SEV revealed the correct molecular sized bands corresponding to the expression of respective proteins; however, no bands were present in pMV101 lanes. 3-actin was used as loading control in all the cases.

FIG. 2, comprising FIG. 2A and FIG. 2B, depicts representative results demonstrating that PCaA-SEV induced potent antigen specific cellular immune responses in mice. FIG. 2A depicts a schematic representation of different time points of EP mediated immunization and immune analysis of this study. C57BL/6 mice (n=4 per group) were immunized with 50 μg of PCaA-SEV or pMV101 using EP mediated enhanced delivery. A week after the third immunization (day 35), mice belonging to all the groups were euthanized and splenocytes were collected for ELISpot assay. FIG. 2B depicts representative results of IFN-γ ELISpot assay, which was performed on splenocytes obtained from mice immunized with PCaA-SEV through EP after ex vivo stimulation with PCaA specific peptides. IFN-γ produced by the cells specific to these antigens are reported as spot forming units (SFUs) per million cells. In case of different PCaA vaccine groups, notably higher cellular immune responses were found to be generated compared to pMV101 group of mice. The graphs represent average IFN-γ SFUs generated per 10⁶ splenocytes+/−SEM for the target peptide. Group average SFUs per million cells are presented.

FIG. 3, comprising FIG. 3A and FIG. 3B, depicts representative results demonstrating that PCaA-SEV induced both CD4⁺ and CD8⁺ T cell responses in mice. Splenocytes obtained from mice, after three immunizations of PCaA-SEV or pMV101, were stimulated with respective PCaA target peptides ex vivo and then stained with different fluorophore-tagged antibodies as shown, for determining the production of cytokines by both CD4⁺ and CD8⁺ T cells. Graphs indicate the total percentage of IFN-γ⁺, TNF-α⁺, and IL-2⁺ T cells (mean±SD) pooled from four mice and two independently performed experiments. PCaA-SEV resulted in higher frequency of CD4+ as well as CD8+ 583 cells secreting intracellular cytokines upon ex vivo stimulation with antigen specific peptides. FIG. 3A depicts representative results demonstrating that splenocytes from PCaA-SEV immunized mice as shown in the previous experiments were also evaluated by polychromatic flow cytometry to measure CD4⁺ T cells producing different cytokines. FIG. 3B depicts representative results demonstrating that splenocytes from PCaA-SEV immunized mice as shown in the previous experiments were also evaluated by polychromatic flow cytometry to measure CD8⁺ T cells producing different cytokines.

FIG. 4, comprising FIG. 4A and FIG. 4B, depicts representative results demonstrating that PCaA-SEV induced humoral immune response. FIG. 4A depicts representative results for ELISA reactive antibodies following the third dose of immunization with PCaA-SEV (day 35). Sera were diluted as shown and vaccine-specific IgG reacting with each antigen was determined through ELISA. Mean optical density and standard error for each group/dilution against each antigen is indicated. FIG. 4B depicts representative results of indirect immunofluorescence analysis of prostate antigen expression in HepG2 cells expressing different PCaA-SEV to confirm whether antibodies induced by the experimental prostate antigen recognized vaccine-transfected cells. 48 hours post transfection of HepG2 cells, incubated with pooled day 35 sera (1:100) from mice immunized with different PCaA-SEV (50 ug/immunization); FITC-tagged anti-mouse IgG secondary antibody (green), and DAPI (blue) were used in this assay, pre-bleed immune serum was used as negative control.

FIG. 5, comprising FIG. 5A through FIG. 5C, depicts representative results demonstrating that PCaA-SEV delayed tumor progression and enhanced survival of prostate cancer bearing mice. FIG. 5A depicts a schematic representation of TRAMP-C2 tumor cell administration and pMV101 or PCaA-SEV administration into C57BL/6 mice. Mice were administered subcutaneously 1.0×10⁶ TRAMP-C2 cells. After TRAMP-C2 tumor challenge in C57BL/6 mice on day 0, mice were immunized with PCaA-SEV (50 μg/immunization) on day 7, 21, and 35 through optimized EP enhanced delivery. FIG. 5B depicts representative assessment of tumor development in control plasmid (pMV101) and PCaA-SEV+TRAMP-C2 cells injected mice. Tumor volumes (mm³) were measured weekly, by a digital caliper, for up to 80 days post tumor administration in mice. Mice inoculated with PCaA-SEV plasmid exhibited delayed tumor growth, as evinced by tumor volume. FIG. 5C depicts representative Kaplan-Meier survival curves of TRAMP-C2 prostate tumor bearing mice immunized with PCaA-SEV or pMV101 vector. Mice immunized with PCaA-SEV were found to exhibit improved survival compared to the pMV101 vaccinated mice.

FIG. 6, comprising FIG. 6A and FIG. 6B, depicts representative results demonstrating that PCaA-SEV promoted T cell recruitment to the tumor microenvironment. Flow cytometric representation of CD8⁺ T cells from the total CD3⁺ and CD45 cells. Mice were immunized with PCaA-SEV, 3 times at 2-week intervals and challenged with TRAMP-C2. Three weeks after tumor inoculation, a paracentesis was performed for analysis of leukocyte subsets by flow cytometry. FIG. 6A depicts representative flow cytometric representation of CD8⁺ T cells from the total CD3⁺ and CD45⁺ cells. FIG. 6B depicts representative results demonstrating enhanced infiltration of anti-tumor CD8⁺ T cells in the tumor microenvironment of mice vaccinated with PCaA-SEV, after inoculation with TRAMP-C2 prostate tumors for 3 weeks.

### DETAILED DESCRIPTION

In one aspect, the present invention provides consensus amino acid sequences of prostate antigens, including prostatic acid phosphatase (PAP), prostate androgen regulated mucin-like protein 1 (PARM1), prostate carcinoma tumor antigen-1 (PCTA), prostate stem cell antigen (PSCA), prostate secretory protein of 94 amino acids (PSP94), and six-transmembrane epithelial antigen of the prostate-1 (STEAP1) antigens. In another aspect, the present invention provides consensus amino acid sequences of prostate antigens that are capable of breaking tolerance in a targeted species, including PAP, PARM, PCTA, PSCA, PSP94, and STEAP antigens. In various embodiments, the consensus amino acid sequences of prostate antigens comprise one or more amino acid sequence that is at least about 60% identical to SEQ ID NO: 2 or a variant or fragment thereof, SEQ ID NO: 4 or a variant or fragment thereof, SEQ ID NO: 6 or a variant or fragment thereof, SEQ ID NO: 8 or a variant or fragment thereof, SEQ ID NO: 10 or a variant or fragment thereof, SEQ ID NO: 12 or a variant or fragment thereof, SEQ ID NO: 14 or a variant or fragment thereof, SEQ ID NO: 16 or a variant or fragment thereof, SEQ ID NO: 18 or a variant or fragment thereof, SEQ ID NO: 20 or a variant or fragment thereof, SEQ ID NO: 22 or a variant or fragment thereof, SEQ ID NO: 24 or a variant or fragment thereof, or any combination thereof.

In one aspect, the present invention also provides nucleic acid molecules that encode one or more consensus amino acid sequences of prostate antigens (e.g., PAP, PARM, PCTA, PSCA, PSP94, and STEAP antigens). In various embodiments, the nucleic acid molecules comprise one or more nucleotide sequence that is at least about 60% identical to SEQ ID NO: 1 or a variant or fragment thereof, SEQ ID NO: 3 or a variant or fragment thereof, SEQ ID NO: 5 or a variant or fragment thereof, SEQ ID NO: 7 or a variant or fragment thereof, SEQ ID NO: 9 or a variant or fragment thereof, SEQ ID NO: 11 or a variant or fragment thereof, SEQ ID NO: 13 or a variant or fragment thereof, SEQ ID NO: 15 or a variant or fragment thereof, SEQ ID NO: 17 or a variant or fragment thereof, SEQ ID NO: 19 or a variant or fragment thereof, SEQ ID NO: 21 or a variant or fragment thereof, SEQ ID NO: 23 or a variant or fragment thereof, or any combination thereof.

In one aspect, the present invention further provides genetic constructs/vectors and vaccines expressing the consensus amino acid sequences of prostate antigens (e.g., PAP, PARM, PCTA, PSCA, PSP94, and STEAP antigens). In one aspect, the present invention further provides genetic constructs/vectors, immunogenic compositions, and vaccines comprising the consensus amino acid sequences of prostate antigens (e.g., PAP, PARM, PCTA, PSCA, PSP94, and STEAP antigens). In various embodiments, the vaccines, immunogenic compositions, proteins, and/or nucleic acid sequences of the present invention can be used to protect against and treat prostate cancer. The vaccines, immunogenic compositions, proteins, and/or nucleic acid sequences of the present invention can elicit both humoral and cellular immune responses that target the antigen. The vaccines, immunogenic compositions, proteins, and/or nucleic acid sequences of the present invention can elicit neutralizing antibodies and immunoglobulin G (IgG) antibodies that are reactive with the antigen. The vaccines, immunogenic compositions, proteins, and/or nucleic acid sequences of the present invention can also elicit a CD8+ T cell response that is reactive to the antigen and produce one or more of interferon-gamma (IFN-γ) and tumor necrosis factor alpha (TNF-α). In one embodiment, the vaccines, immunogenic compositions, proteins, and/or nucleic acid sequences of the present invention can also elicit a CD4+ T cell response that is reactive to the antigen and produce one or more of IFN-γ and TNF-α. Thus, in one aspect, the present invention provides methods for generating an autoimmune response against prostate cancer cells by administering one or more of the vaccines, immunogenic compositions, proteins, and/or nucleic acid sequences of the present invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value, such as an amount, a temporal duration, and the like, is meant to encompass variations of 20%, ±10%, +5%, +1%, or +0.1% from the specified value, as such variations are appropriate to perform the disclosed methods.

“Consensus” or “consensus sequence” as used herein means a polypeptide sequence based on analysis of an alignment of multiple subtypes of a particular prostate antigen. Nucleic acid sequences that encode a consensus polypeptide sequence may be prepared. Vaccines comprising proteins that comprise consensus sequences and/or nucleic acid molecules that encode such proteins can be used to induce broad immunity against a particular prostate antigen.

A “peptide,” “protein,” or “polypeptide” as used herein can mean a linked sequence of amino acids and can be natural, synthetic, or a modification or combination of natural and synthetic.

“Signal peptide” and “leader sequence” are used interchangeably herein and refer to an amino acid sequence that can be linked at the amino terminus of a tumor microenvironment protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid can be used for the same purpose as a given nucleic acid. Thus, a nucleic acid also encompasses substantially identical nucleic acids and complements thereof. A single strand provides a probe that can hybridize to a target sequence under stringent hybridization conditions. Thus, a nucleic acid also encompasses a probe that hybridizes under stringent hybridization conditions. Nucleic acids can be single stranded or double stranded, or can contain portions of both double stranded and single stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides”. The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the term “genetic construct” refers to the DNA or RNA molecules that comprise a nucleotide sequence which encodes a protein. The coding sequence includes initiation and termination signals operably linked to regulatory elements including a promoter and polyadenylation signal capable of directing expression in the cells of the subject to whom the nucleic acid molecule is administered. As used herein, the term “expressible form” refers to gene constructs that contain the necessary regulatory elements operable linked to a coding sequence that encodes a protein such that when present in the cell of the subject, the coding sequence will be expressed.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of a subject or mammal to whom the nucleic acid is administered.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting there from. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Promoter” as used herein means a synthetic or naturally-derived molecule which is capable of conferring, activating or enhancing expression of a nucleic acid in a cell. A promoter can comprise one or more specific transcriptional regulatory sequences to further enhance expression and/or to alter the spatial expression and/or temporal expression of same. A promoter can also comprise distal enhancer or repressor elements, which can be located as much as several thousand base pairs from the start site of transcription. A promoter can be derived from sources including viral, bacterial, fungal, plants, insects, and animals. A promoter can regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, S V40 early promoter or SV40 late promoter and the CMV IE promoter.

As used herein, the term “expressible form” refers to gene constructs that contain the necessary regulatory elements operably linked to a coding sequence that encodes a target protein or an immunomodulating protein, such that when present in the cell of the individual, the coding sequence will be expressed.

“Operably linked” as used herein means that expression of a gene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function. n. Promoter

As used herein, “linked” means to couple directly or indirectly one molecule with another by whatever means, e.g., by covalent bonding, by non-covalent bonding, by ionic bonding, or by non-ionic bonding. Covalent bonding includes bonding by various linkers such as thioether linkers or thioester linkers. Direct linking involves one molecule attached to the molecule of interest. Indirect linking involves one molecule attached to another molecule which in turn is attached directly or indirectly to the molecule of interest.

“Stringent hybridization conditions” as used herein means conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions can be selected to be about 5-10° C. lower than the thermal melting point (Tm) for the specific sequence at a defined ionic strength pH. The Tm can be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions can be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions can also be achieved with the addition of destabilizing agents such as formamide. For selective or specific hybridization, a positive signal can be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein means that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 180, 270, 360, 450, 540, 630, 720, 810, 900, 990, 1080, 1170, 1260, 1350, 1440, 1530, 1620, 1710, 1800, 1890, 1980, 2070 or more nucleotides or amino acids, or that the two sequences hybridize under stringent hybridization conditions.

“Complement” or “complementary” as used herein means a nucleic acid can mean Watson-Crick (e.g., A-T/U and C-G) or Hoogsteen base pairing between nucleotides or nucleotide analogs of nucleic acid molecules.

The term “recombinant DNA” as used herein is defined as DNA produced by joining pieces of DNA from different sources.

The term “recombinant RNA” as used herein is defined as RNA produced by joining pieces of RNA from different sources.

“Identical” or “identity” as used herein in the context of two or more nucleic acids or polypeptide sequences, means that the sequences have a specified percentage of residues that are the same over a specified region. The percentage can be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) can be considered equivalent. Identity can be performed manually or by using a computer sequence algorithm such as BLAST or BLAST 2.0.

The term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, “substantially identical” as used herein can mean that a first and second amino acid sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1170, 1260, 1350, 1440, 1530, 1620, 1710, 1800, 1890, 1980, 2070 or more amino acids. Substantially identical can also mean that a first nucleotide sequence and a second nucleotide sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1164, 1170, 1260, 1350, 1440, 1530, 1620, 1710, 1800, 1890, 1980, 2070 or more nucleotides. The identity of a sequence can exist over a region that is at least about 75-100 sequential units in length, over a region that is about 50 sequential units in length, or, where not specified, across the entire sequence. This definition also refers to the complement of a test sequence.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

Variant can further be defined as a peptide or polypeptide that differs in amino acid sequence by the insertion, deletion, or conservative substitution of amino acids, but retain at least one biological activity. Representative examples of “biological activity” include the ability to be bound by a specific antibody or to promote an immune response. Variant can also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of 2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. Substitution of amino acids having similar hydrophilicity values can result in peptides retaining biological activity, for example immunogenicity, as is understood in the art. Substitutions can be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleotide sequence that is substantially identical over the full length of the full gene sequence or a fragment thereof. The nucleotide sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Fragment” as used herein with respect to nucleic acid sequences means a nucleic acid sequence or a portion thereof, that encodes a polypeptide capable of eliciting an immune response in a mammal that cross reacts with a full length prostate antigen. For example, in one embodiment, the term “fragment” refers to a portion of the variable region of the immunoglobulin molecule which binds to its target, i.e. the antigen binding region. Some of the constant region of the immunoglobulin may be included. The fragments can be DNA fragments selected from at least one of the various nucleotide sequences that encode the consensus amino acid sequences and constructs comprising such sequences. DNA fragments can comprise coding sequences for the immunoglobulin leader such as IgE or IgG sequences. DNA fragments can encode the protein fragments set forth below.

“Fragment” as used herein means a nucleotide sequence or a portion thereof that encodes a polypeptide capable of eliciting an immune response in a mammal. The fragments can be DNA fragments selected from at least one of the various nucleotide sequences that encode protein fragments set forth below. “Fragment” or “immunogenic fragment” with respect to polypeptide sequences means a polypeptide capable of eliciting an immune response in a mammal that cross reacts with a full length endogenous antigen. Fragments of consensus proteins can comprise at least 10%, at least 20%, at least 30%, at least 40%, at least 50%, at least 60%, at least 70%, at least 80%, at least 90% or at least 95% of a consensus protein. In some embodiments, fragments of consensus proteins can comprise at least 20 amino acids or more, at least 30 amino acids or more, at least 40 amino acids or more, at least 50 amino acids or more, at least 60 amino acids or more, at least 70 amino acids or more, at least 80 amino acids or more, at least 90 amino acids or more, at least 100 amino acids or more, at least 110 amino acids or more, at least 120 amino acids or more, at least 130 amino acids or more, at least 140 amino acids or more, at least 150 amino acids or more, at least 160 amino acids or more, at least 170 amino acids or more, at least 180 amino acids or more, at least 190 amino acids or more, at least 200 amino acids or more, at least 210 amino acids or more, at least 220 amino acids or more, at least 230 amino acids or more, at least 240 amino acids or more of a consensus protein or at least 340 amino acids or more of a consensus protein. “Fragment” with respect to polypeptide sequences means a polypeptide capable of eliciting an immune response in a mammal that cross reacts with a prostate antigen, including, e.g. PAP, PARM1, PCTA, PSCA, PSP94, and STEAP1.

“Subtype” or “serotype”: as used herein, interchangeably, and in reference to prostate cancer antigens, means genetic variants of a prostate cancer antigen such that one subtype (or variant) is recognized by an immune system apart from a different subtype.

“Vector” as used herein means a nucleic acid sequence containing an origin of replication. A vector can be a viral vector, bacteriophage, bacterial artificial chromosome or yeast artificial chromosome. A vector can be a DNA or RNA vector. A vector can be a self-replicating extrachromosomal vector, and preferably, is a DNA plasmid.

“Adjuvant” as used herein means any molecule added to the DNA plasmid vaccines described herein to enhance the immunogenicity of the antigens encoded by the DNA plasmids and the encoding nucleic acid sequences described hereinafter.

The term “antibody,” as used herein, refers to an immunoglobulin molecule which is able to specifically bind to a specific epitope of an antigen. The antibody can be intact immunoglobulins derived from natural sources, or from recombinant sources and can be immunoreactive portions of intact immunoglobulins. The antibody can be an antibody isolated from the serum sample of mammal, a polyclonal antibody, affinity purified antibody, or mixtures thereof which exhibits sufficient binding specificity to a desired epitope or a sequence derived therefrom. The antibody in the present invention may exist in a variety of forms including, for example, polyclonal antibodies, monoclonal antibodies, intracellular antibodies (“intrabodies”), Fv, Fab, Fab′, F(ab)2 and F(ab′)2, as well as single chain antibodies (scFv), heavy chain antibodies, such as camelid antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, New York; Houston et al., 1988, Proc. Natl. Acad. Sci. USA 85:5879-5883; Bird et al., 1988, Science 242:423-426). “Antibody” as used herein means an antibody of classes IgG, IgM, IgA, IgD or IgE, or fragments, fragments or derivatives thereof, including Fab, F(ab′)₂, Fd, and single chain antibodies, diabodies, bispecific antibodies, bifunctional antibodies and derivatives thereof.

The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen.

As used herein, “antigen-binding domain” means that part of the antibody, recombinant molecule, the fusion protein, or the immunoconjugate of the invention which recognizes the target or portions thereof.

By the term “specifically binds,” as used herein, is meant a molecule, such as an antibody, which recognizes and binds to another molecule or feature, but does not substantially recognize or bind other molecules or features in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, the terms “targeting domain”, “targeting moiety”, or “targeting group” are used interchangeably and refer to all molecules capable of specifically binding to a particular target molecule and forming a bound complex as described above. Thus, the ligand and its corresponding target molecule form a specific binding pair.

The terms “effective amount” and “pharmaceutically effective amount” refer to a sufficient amount of an agent to provide the desired biological result. That result can be reduction and/or alleviation of the signs, symptoms, or causes of a disease or disorder (e.g., prostate cancer), or any other desired alteration of a biological system. An appropriate effective amount in any individual case may be determined by one of ordinary skill in the art using routine experimentation. An “effective amount” or “therapeutically effective amount” of a compound is that amount of compound, which is sufficient to provide a beneficial effect to the subject to which the compound is administered.

As used herein, “pharmaceutically-acceptable” means that drugs, medicaments or inert ingredients which the term describes are suitable for use in contact with the tissues of humans and lower animals without undue toxicity, incompatibility, instability, irritation, allergic response, and the like, commensurate with a reasonable benefit/risk ratio.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the terms “therapeutic compound”, “therapeutic agent”, “drug”, “active pharmaceutical”, and “active pharmaceutical ingredient” are used interchangeably to refer to chemical entities that display certain pharmacological effects in a body and are administered for such purpose. Non-limiting examples of therapeutic agents include, but are not limited to, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, metabolites, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents. In certain embodiments, the one or more therapeutic agents are water-soluble, poorly water-soluble drug or a drug with a low, medium or high melting point. The therapeutic agents may be provided with or without a stabilizing salt or salts.

Some examples of active ingredients suitable for use in the pharmaceutical formulations and methods of the present invention include: hydrophilic, lipophilic, amphiphilic or hydrophobic, and that can be solubilized, dispersed, or partially solubilized and dispersed, on or about the microparticle cluster. The active agent-microparticle cluster combination may be coated further to encapsulate the agent-microparticle cluster combination and may be directed to a target by functionalizing the microparticle cluster with, e.g., aptamers and/or antibodies. Alternatively, an active ingredient may also be provided separately from the solid pharmaceutical composition, such as for co-administration. Such active ingredients can be any compound or mixture of compounds having therapeutic or other value when administered to an animal, particularly to a mammal, such as drugs, nutrients, cosmeceuticals, nutraceuticals, diagnostic agents, nutritional agents, and the like. The active agents described herein may be found in their native state, however, they will generally be provided in the form of a salt. The active agents described herein include their isomers, analogs and derivatives.

The term “solvate” in accordance with this invention should be understood as meaning any form of the active compound in accordance with the invention in which the said compound is bonded by a non-covalent bond to another molecule (normally a polar solvent), including especially hydrates and alcoholates.

As used herein, the term “stabilizers” refers to either, or both, primary particle and/or secondary stabilizers, which may be polymers or other small molecules. Non-limiting examples of primary particle and/or secondary stabilizers for use with the present invention include, e.g., starch, modified starch, and starch derivatives, gums, including but not limited to polymers, polypeptides, albumin, amino acids, thiols, amines, carboxylic acid and combinations or derivatives thereof. Other examples include xanthan gum, alginic acid, other alginates, benitoniite, veegum, agar, guar, locust bean gum, gum arabic, quince psyllium, flax seed, okra gum, arabinoglactin, pectin, tragacanth, scleroglucan, dextran, amylose, amylopectin, dextrin, etc., cross-linked polyvinylpyrrolidone, ion-exchange resins, potassium polymethacrylate, carrageenan (and derivatives), gum karaya and biosynthetic gum. Other examples of useful primary particle and/or secondary stabilizers include polymers such as: polycarbonates (linear polyesters of carbonic acid); microporous materials (bisphenol, a microporous poly(vinylchloride), micro-porous polyamides, microporous modacrylic copolymers, microporous styrene-acrylic and its copolymers); porous polysulfones, halogenated poly(vinylidene), polychloroethers, acetal polymers, polyesters prepared by esterification of a dicarboxylic acid or anhydride with an alkylene polyol, poly(alkylenesulfides), phenolics, polyesters, asymmetric porous polymers, cross-linked olefin polymers, hydrophilic microporous homopolymers, copolymers or interpolymers having a reduced bulk density, and other similar materials, poly(urethane), cross-linked chain-extended poly(urethane), poly(mides), poly(benzimidazoles), collodion, regenerated proteins, semi-solid cross-linked poly(vinylpyrrolidone).

A “disease” is a state of health of a subject wherein the subject cannot maintain homeostasis, and wherein if the disease is not ameliorated then the subject's health continues to deteriorate.

In contrast, a “disorder” in a subject is a state of health in which the subject is able to maintain homeostasis, but in which the subject's state of health is less favorable than it would be in the absence of the disorder. Left untreated, a disorder does not necessarily cause one decrease in the subject's state of health.

The terms “patient”, “subject”, “individual”, and the like are used interchangeably herein, and refer to any animal, in some embodiments a mammal, and in some embodiments a human, having a complement system, including a human in need of therapy for, or susceptible to, a condition or its sequelae. As used herein, the terms “patient”, “subject”, “individual”, and the like can be a vertebrate, such as a mammal, a fish, a bird, a reptile, or an amphibian. Thus, the subject of the herein disclosed methods can be a human, non-human primate, monkey, horse, pig, rabbit, dog, sheep, goat, cow, cat, mouse, rat, guinea pig or rodent. The terms do not denote a particular age or sex. Thus, adult and newborn subjects, as well as fetuses, whether male or female, are intended to be covered. In one aspect, the subject is a mammal.

“Immune response” as used herein means the activation of a host's immune system, e.g., that of a mammal, in response to the introduction of antigen, such as a prostate consensus antigen. The immune response can be in the form of a cellular or humoral response, or both.

“Treatment” or “treating,” as used herein can mean protecting of a subject from a prostate cancer through means of preventing, suppressing, repressing, or completely eliminating the prostate cancer. In one embodiment, preventing the prostate cancer involves administering an immunogenic composition (e.g., vaccine) of the present invention to a subject prior to onset of the prostate cancer. In one embodiment, preventing the prostate cancer involves administering an immunogenic composition of the present invention to a subject following a treatment so as to prevent reoccurrence or further progression of the prostate cancer. Suppressing the prostate cancer involves administering an immunogenic composition of the present invention to a subject after induction of the prostate cancer but before its clinical appearance. Repressing the prostate cancer involves administering an immunogenic composition of the present invention to a subject after clinical appearance of the prostate cancer.

A “therapeutic treatment” is a treatment administered to a subject who exhibits signs of prostate cancer, for the purpose of diminishing or eliminating those signs.

As used herein, “treating a prostate cancer” means reducing the frequency and/or severity of a sign and/or symptom of the prostate cancer is experienced by a subject.

A prostate cancer is “alleviated” if the severity of a sign or symptom of the prostate cancer, the frequency with which such a sign or symptom is experienced by a subject, or both, is reduced.

“Electroporation,” “electro-permeabilization,” or “electro-kinetic enhancement” (“EP”) as used interchangeably herein means the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range, such as from 1 to 6, should be considered to have specifically disclosed subranges, such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides consensus prostate protein sequences and isolated nucleic acid molecules that encode them, and in particular, the prostatic acid phosphatase (PAP), prostate androgen regulated mucin-like protein 1 (PARM1), prostate carcinoma tumor antigen-1 (PCTA), prostate stem cell antigen (PSCA), prostate secretory protein of 94 amino acids (PSP94), and six-transmembrane epithelial antigen of the prostate-1 (STEAP1) antigens. The prostate cancer antigens described herein are consensus sequences derived from a pool of homologous antigens from across multiple species, including the species that the immunogenic composition and/or vaccine is targeted for. The selected species from which antigen sequences are aligned to form a consensus shall be chosen based on close proximity of the species on a phylogenic tree, e.g., H. sapiens (humans), M. mulatta (rhesus macaques), and M. fascicularis (cynomolgus monkey). The consensus antigen is not identical to the native prostate antigen but will have close identity, which sequences share at least 60%, and preferably 85%, 86%, 87%, 88%, 89%, 90%, 91%, 92%, 93%, 94%, 95%, 96%, 97%, 98%, or 99% identity. These described consensus cancer antigens are able to break tolerance in the targeted specie (or cause autoimmunity) and generate an effective immune response against the prostate cancer antigen. Provided herein are methods to generate a consensus cancer antigen based DNA vaccine.

In one aspect, the present invention provides consensus prostate protein sequences selected from a PAP consensus antigen, PARM1 consensus antigen, PCTA consensus antigen, PSCA consensus antigen, PSP94 consensus antigen, STEAP consensus antigen, or any combination thereof. Two consensus protein sequences for PAP are disclosed: PAP Consensus Antigen sequence 1 (SEQ ID NO: 2) and PAP Consensus Antigen sequence 2 lacking start and stop codon (SEQ ID NO: 4). Two consensus protein sequences for PARM1 are disclosed: PARM1 Consensus Antigen sequence 1 (SEQ ID NO: 6) and PARM1 Consensus Antigen sequence 2 lacking start and stop codon (SEQ ID NO: 8). Two consensus protein sequences for PCTA are disclosed: PCTA Consensus Antigen sequence 1 (SEQ ID NO: 10) and PCTA Consensus Antigen sequence 2 lacking start and stop codon (SEQ ID NO: 12). Two consensus protein sequences for PSCA are disclosed: PSCA Consensus Antigen sequence 1 (SEQ ID NO: 14) and PSCA Consensus Antigen sequence 2 lacking start and stop codon (SEQ ID NO: 16). Two consensus protein sequences for PSP94 are disclosed: PSP94 Consensus Antigen sequence 1 (SEQ ID NO: 18) and PSP94 Consensus Antigen sequence 2 lacking start and stop codon (SEQ ID NO: 20). Two consensus protein sequences for STEAP (also referred to herein as STEAP1) are disclosed: STEAP Consensus Antigen sequence 1 (SEQ ID NO: 22) and STEAP Consensus Antigen sequence 2 lacking start and stop codon (SEQ ID NO: 24).

Thus, in various aspects, the present invention relates, in part, to consensus proteins comprising one or more proteins selected from the group comprising: a) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, b) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, c) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, d) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, e) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, f) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, g) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, h) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, i) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, j) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, k) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and l) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments the consensus proteins are chosen from ones comprising proteins a), b), c), d), e), f), g), h), i), j), k), and/or l), above. In other embodiments the consensus proteins are ones comprising one or more proteins selected from at least one selected from ones comprising either proteins a) or b), at least one selected from ones comprising either proteins c) or d), at least one selected from ones comprising either proteins e) or f), at least one selected from ones comprising either proteins g) or h), at least one selected from ones encoding either proteins i) or j), and/or at least one selected from ones comprising either proteins k) or l).

In some embodiments, the consensus proteins comprise one or more proteins selected from the group comprising: a) an amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, b) an amino acid sequence having at least about 90% identity over an entire length of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, c) an immunogenic fragment comprising at least about 90% identity over at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, and/or d) an immunogenic fragment comprising at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof.

For example, in some embodiments, the consensus proteins can be ones that comprise one or more proteins selected from SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof.

In some embodiments, the PAP Consensus Antigen sequence 2 lacking start and stop codon (SEQ ID NO: 4) may be linked to an IgE signal peptide sequence (SEQ ID NO: 26) or a signal sequence other than the IgE signal peptide sequence. In some embodiments, the PARM1 Consensus Antigen sequence 2 lacking start and stop codon (SEQ ID NO: 8) may be linked to an IgE signal peptide sequence (SEQ ID NO: 26) or a signal sequence other than the IgE signal peptide sequence. In some embodiments, the PCTA Consensus Antigen sequence 2 lacking start and stop codon (SEQ ID NO: 12) may be linked to an IgE signal peptide sequence (SEQ ID NO: 26) or a signal sequence other than the IgE signal peptide sequence. In some embodiments, the PSCA Consensus Antigen sequence 2 lacking start and stop codon (SEQ ID NO: 16) may be linked to an IgE signal peptide sequence (SEQ ID NO: 26) or a signal sequence other than the IgE signal peptide sequence. In some embodiments, the PSP94 Consensus Antigen sequence 2 lacking start and stop codon (SEQ ID NO: 20) may be linked to an IgE signal peptide sequence (SEQ ID NO: 26) or a signal sequence other than the IgE signal peptide sequence. In some embodiments, the STEAP1 Consensus Antigen sequence 2 lacking start and stop codon (SEQ ID NO: 24) may be linked to an IgE signal peptide sequence (SEQ ID NO: 26) or a signal sequence other than the IgE signal peptide sequence.

In one aspect, the present invention relates, in part, to nucleic acid molecules comprising a coding sequence encoding one or more proteins selected from the group comprising: a) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, b) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, c) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, d) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, e) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, f) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, g) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, h) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, i) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, j) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, k) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and l) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

In some embodiments the nucleic acid molecules are chosen from ones encoding proteins a), b), c), d), e), f), g), h), i), j), k), and/or l), above. In other embodiments the nucleic acid molecules are ones encoding one or more proteins selected from the group comprising: at least one selected from ones encoding either proteins a) or b), at least one selected from ones encoding either proteins c) or d), at least one selected from ones encoding either proteins e) or f), at least one selected from ones encoding either proteins g) or h), at least one selected from ones encoding either proteins i) or j), and/or at least one selected from ones encoding either proteins k) or l).

In some embodiments, the nucleic acid molecules comprise a coding sequence encoding one or more proteins selected from the group comprising: a) an amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, b) an amino acid sequence having at least about 90% identity over an entire length of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, c) an immunogenic fragment comprising at least about 90% identity over at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof, and/or d) an immunogenic fragment comprising at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof.

For example, in some embodiments, the nucleic acid molecule can be ones that encode one or more proteins selected from SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof.

In one aspect, the present invention relates, in part, to nucleic acid molecules comprising nucleic acid coding sequences that have been generated to improve and optimize expression. The codons used in these nucleic acid molecules were selected to generate RNA having reduced secondary structure formation due to intramolecular hybridization. Nucleic acid sequences encoding PAP consensus antigen, PARM1 consensus antigen, PCTA consensus antigen, PSCA consensus antigen, PSP94 consensus antigen, STEAP consensus antigen, or any combination thereof, are disclosed. Two consensus nucleotide sequences for PAP are disclosed: PAP Consensus Antigen sequence 1 (SEQ ID NO: 1) and PAP Consensus Antigen sequence 2 lacking start and stop codon (SEQ ID NO: 3). Two consensus nucleotide sequences for PARM1 are disclosed: PARM1 Consensus Antigen sequence 1 (SEQ ID NO: 5) and PARM1 Consensus Antigen sequence 2 lacking start and stop codon (SEQ ID NO: 7). Two consensus nucleotide sequences for PCTA are disclosed: PCTA Consensus Antigen sequence 1 (SEQ ID NO: 9) and PCTA Consensus Antigen sequence 2 lacking start and stop codon (SEQ ID NO: 11). Two consensus nucleotide sequences for PSCA are disclosed: PSCA Consensus Antigen sequence 1 (SEQ ID NO: 13) and PSCA Consensus Antigen sequence 2 lacking start and stop codon (SEQ ID NO: 15). Two consensus nucleotide sequences for PSP94 are disclosed: PSP94 Consensus Antigen sequence 1 (SEQ ID NO: 17) and PSP94 Consensus Antigen sequence 2 lacking start and stop codon (SEQ ID NO: 19). Two consensus nucleotide sequences for STEAP (also referred to herein as STEAP1) are disclosed: STEAP Consensus Antigen sequence 1 (SEQ ID NO: 21) and STEAP Consensus Antigen sequence 2 lacking start and stop codon (SEQ ID NO: 23).

Thus, in various aspects, the present invention relates, in part, to nucleic acid molecules comprising one or more nucleotide sequences selected from the group comprising: a) SEQ ID NO: 1, a nucleotide sequence that is at least about 90% homologous to SEQ ID NO: 1, an immunogenic fragment of SEQ ID NO: 1, an immunogenic fragment of a nucleotide sequence that is at least about 90% homologous to SEQ ID NO: 1, or any combination thereof, b) SEQ ID NO: 3, a nucleotide sequence that is at least about 90% homologous to SEQ ID NO: 3, an immunogenic fragment of SEQ ID NO: 3, an immunogenic fragment of a nucleotide sequence that is at least about 90% homologous to SEQ ID NO: 3, or any combination thereof, c) SEQ ID NO: 5, a nucleotide sequence that is at least about 90% homologous to SEQ ID NO: 5, an immunogenic fragment of SEQ ID NO: 5, an immunogenic fragment of a nucleotide sequence that is at least about 90% homologous to SEQ ID NO: 5, or any combination thereof, d) SEQ ID NO: 7, a nucleotide sequence that is at least about 90% homologous to SEQ ID NO: 7, an immunogenic fragment of SEQ ID NO: 7, an immunogenic fragment of a nucleotide sequence that is at least about 90% homologous to SEQ ID NO: 7, or any combination thereof, e) SEQ ID NO: 9, a nucleotide sequence that is at least about 90% homologous to SEQ ID NO: 9, an immunogenic fragment of SEQ ID NO: 9, an immunogenic fragment of a nucleotide sequence that is at least about 90% homologous to SEQ ID NO: 9, or any combination thereof, f) SEQ ID NO: 11, a nucleotide sequence that is at least about 90% homologous to SEQ ID NO: 11, an immunogenic fragment of SEQ ID NO: 11, an immunogenic fragment of a nucleotide sequence that is at least about 90% homologous to SEQ ID NO: 11, or any combination thereof, g) SEQ ID NO: 13, a nucleotide sequence that is at least about 90% homologous to SEQ ID NO: 13, an immunogenic fragment of SEQ ID NO: 13, an immunogenic fragment of a nucleotide sequence that is at least about 90% homologous to SEQ ID NO: 13, or any combination thereof, h) SEQ ID NO: 15, a nucleotide sequence that is at least about 90% homologous to SEQ ID NO: 15, an immunogenic fragment of SEQ ID NO: 15, an immunogenic fragment of a nucleotide sequence that is at least about 90% homologous to SEQ ID NO: 15, or any combination thereof, i) SEQ ID NO: 17, a nucleotide sequence that is at least about 90% homologous to SEQ ID NO: 17, an immunogenic fragment of SEQ ID NO: 17, an immunogenic fragment of a nucleotide sequence that is at least about 90% homologous to SEQ ID NO: 17, or any combination thereof, j) SEQ ID NO: 19, a nucleotide sequence that is at least about 90% homologous to SEQ ID NO:20, an immunogenic fragment of SEQ ID NO: 19, an immunogenic fragment of a nucleotide sequence that is at least about 90% homologous to SEQ ID NO: 19, or any combination thereof, k) SEQ ID NO: 21, a nucleotide sequence that is at least about 90% homologous to SEQ ID NO: 21, an immunogenic fragment of SEQ ID NO: 21, an immunogenic fragment of a nucleotide sequence that is at least about 90% homologous to SEQ ID NO: 21, or any combination thereof, and l) SEQ ID NO: 23, a nucleotide sequence that is at least about 90% homologous to SEQ ID NO: 23, an immunogenic fragment of SEQ ID NO: 23, an immunogenic fragment of a nucleotide sequence that is at least about 90% homologous to SEQ ID NO: 23, or any combination thereof.

In some embodiments the nucleic acid molecules are chosen from ones comprising nucleotide sequences a), b), c), d), e), f), g), h), i), j), k), and/or l), above. In other embodiments the nucleic acid molecules are ones comprising one or more nucleotide sequences selected from at least one selected from ones comprising either nucleotide sequences a) or b), at least one selected from ones comprising either nucleotide sequences c) or d), at least one selected from ones comprising either nucleotide sequences e) or f), at least one selected from ones comprising either nucleotide sequences g) or h), at least one selected from ones encoding either nucleotide sequences i) or j), and/or at least one selected from ones comprising either nucleotide sequences k) or l).

In some embodiments, the nucleic acid molecules comprise one or more nucleotide sequences selected from the group comprising: a) a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof, b) a nucleotide sequence having at least about 90% identity over an entire length of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof, c) an immunogenic fragment comprising at least about 90% identity over at least 60% of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof, and/or d) an immunogenic fragment comprising at least 60% of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof.

For example, in some embodiments, the nucleic acid molecules can be ones that comprise one or more nucleotide sequences selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof.

In some embodiments, the nucleotide sequence lacking start and stop codon for PAP Consensus Antigen sequence 2 (SEQ ID NO: 3) may be linked to a nucleotide sequence for an IgE signal peptide (SEQ ID NO: 25) or a nucleotide sequence for a signal peptide other than the IgE signal peptide. In some embodiments, the nucleotide sequence lacking start and stop codon PARM1 Consensus Antigen sequence 2 (SEQ ID NO: 7) may be linked to a nucleotide sequence for an IgE signal peptide (SEQ ID NO: 25) or a nucleotide sequence for a signal peptide other than the IgE signal peptide. In some embodiments, the nucleotide sequence lacking start and stop codon for PCTA Consensus Antigen sequence 2 (SEQ ID NO: 11) may be linked to a nucleotide sequence for an IgE signal peptide (SEQ ID NO: 25) or a nucleotide sequence for a signal peptide other than the IgE signal peptide. In some embodiments, the nucleotide sequence lacking start and stop codon for PSCA Consensus Antigen sequence 2 (SEQ ID NO: 15) may be linked to a nucleotide sequence for an IgE signal peptide (SEQ ID NO: 25) or a nucleotide sequence for a signal peptide other than the IgE signal peptide. In some embodiments, the nucleotide sequence lacking start and stop codon for PSP94 Consensus Antigen sequence 2 (SEQ ID NO: 19) may be linked to a nucleotide sequence for an IgE signal peptide (SEQ ID NO: 25) or a nucleotide sequence for a signal peptide other than the IgE signal peptide. In some embodiments, the nucleotide sequence lacking start and stop codon for STEAP1 Consensus Antigen sequence 2 (SEQ ID NO: 23) may be linked to a nucleotide sequence for an IgE signal peptide (SEQ ID NO: 25) or a nucleotide sequence for a signal peptide other than the IgE signal peptide.

Thus, in some embodiments, the nucleic acid molecules of the present invention comprises one or more nucleotide sequences selected from the group comprising: a) a nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof linked to a nucleotide sequence for an IgE signal peptide (SEQ ID NO: 25) or a nucleotide sequence for a signal peptide other than the IgE signal peptide; b) a nucleotide sequence having at least about 90% identity over an entire length of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof linked to a nucleotide sequence for an IgE signal peptide (SEQ ID NO: 25) or a nucleotide sequence for a signal peptide other than the IgE signal peptide; c) an immunogenic fragment comprising at least about 90% identity over at least 60% of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof linked to a nucleotide sequence for an IgE signal peptide (SEQ ID NO: 25) or a nucleotide sequence for a signal peptide other than the IgE signal peptide; and/or d) an immunogenic fragment comprising at least 60% of the nucleotide sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, or any combination thereof linked to a nucleotide sequence for an IgE signal peptide (SEQ ID NO: 25) or a nucleotide sequence for a signal peptide other than the IgE signal peptide.

In one aspect, the preset invention provides compositions comprising one or more consensus proteins described herein. In one aspect, the preset invention provides compositions comprising one or more nucleic acid molecules described herein. In various embodiments, the compositions of the present invention may be useful for inducing immune responses against a prostate protein when administered into a subject. For example, in some embodiments, the composition of the present invention comprising one or more consensus proteins may be useful for inducing immune responses against a prostate protein when administered into a subject. In some embodiments, the composition of the present invention comprising the nucleic acid molecules which comprise the coding sequences of the isolated nucleic acid molecules provided herein may be useful for inducing immune responses against a prostate protein when administered into a subject. Thus, in various embodiments, the compositions of the present invention are immunogenic compositions. In other embodiments, the compositions of the present invention are vaccines.

Compositions containing one or more of these nucleic acid sequences may be used as vaccines or vaccine components to prophylactically or therapeutically immunize against prostate cancer. Likewise, compositions comprising consensus proteins may be useful for inducing immune responses against a prostate protein when administered into a subject. Combinations of compositions comprising nucleic acid molecules which comprise the coding sequences of the isolated nucleic acid molecules provided herein may be useful to induce immune responses against a prostate protein and may collectively be used as vaccines or vaccine components to prophylactically or therapeutically immunize against prostate cancer. Likewise, combinations of compositions comprising consensus proteins may be useful for inducing immune responses against a prostate protein when administered into a subject. Compositions containing one or more of these consensus proteins may be used as vaccines or vaccine components to prophylactically or therapeutically immunize against prostate cancer.

In various aspects, the present invention also provides vaccines comprising nucleic acid sequences provided herein. In some embodiments, vaccines are provided which comprises nucleic acid sequences encoding one or more consensus prostate antigens selected from a consensus prostate antigen selected from a consensus PAP antigen 1, consensus PAP antigen 2 lacking start and stop codon, consensus PARM1 antigen 1, consensus PARM1 antigen 2 lacking start and stop codon, consensus PCTA antigen 1, consensus PCTA antigen 2 lacking start and stop codon, consensus PSCA antigen 1, consensus PSCA antigen 2 lacking start and stop codon, consensus PSP94 antigen 1, consensus PSP94 antigen 2 lacking start and stop codon, consensus STEAP antigen 1, consensus STEAP antigen 2 lacking start and stop codon, or any combination thereof. Thus, methods of inducing immune responses using nucleic acid sequences encoding one or more prostate antigens selected from the group consisting of: a consensus PAP antigen 1, consensus PAP antigen 2 lacking start and stop codon, consensus PARM1 antigen 1, consensus PARM1 antigen 2 lacking start and stop codon, consensus PCTA antigen 1, consensus PCTA antigen 2 lacking start and stop codon, consensus PSCA antigen 1, consensus PSCA antigen 2 lacking start and stop codon, consensus PSP94 antigen 1, consensus PSP94 antigen 2 lacking start and stop codon, consensus STEAP antigen 1, consensus STEAP antigen 2 lacking start and stop codon, or any combination thereof are provided.

In various aspects, the present invention also provides vaccines comprising consensus proteins provided herein. In some embodiments, vaccines are provided which comprise consensus prostate antigen comprising one or more selected from a consensus prostate antigen selected from a consensus PAP antigen 1, consensus PAP antigen 2 lacking start and stop codon, consensus PARM1 antigen 1, consensus PARM1 antigen 2 lacking start and stop codon, consensus PCTA antigen 1, consensus PCTA antigen 2 lacking start and stop codon, consensus PSCA antigen 1, consensus PSCA antigen 2 lacking start and stop codon, consensus PSP94 antigen 1, consensus PSP94 antigen 2 lacking start and stop codon, consensus STEAP antigen 1, consensus STEAP antigen 2 lacking start and stop codon, or any combination thereof. Thus, methods of inducing immune responses using one or more of consensus PAP antigen 1, consensus PAP antigen 2 lacking start and stop codon, consensus PARM1 antigen 1, consensus PARM1 antigen 2 lacking start and stop codon, consensus PCTA antigen 1, consensus PCTA antigen 2 lacking start and stop codon, consensus PSCA antigen 1, consensus PSCA antigen 2 lacking start and stop codon, consensus PSP94 antigen 1, consensus PSP94 antigen 2 lacking start and stop codon, consensus STEAP antigen 1, consensus STEAP antigen 2 lacking start and stop codon, or any combination thereof are also provided.

In various aspects, the present invention also relates, in part, to methods of protecting a subject against prostate cancer or of treating a subject who has been identified as having prostate cancer are provided. The methods comprise the step of. administering to said subject an effective amount of one or more nucleic acid molecules comprising one or more nucleic acid sequences provided herein. In some methods, the delivery of the nucleic acid molecules is facilitated by electroporation of the targeted tissue or the tissue that receives the nucleic acid molecules. The nucleic acid sequence is expressed in cells of the subject and an immune response is induced against the prostate protein encoded by the nucleic acid sequence.

### Consensus Prostate Antigens

The present invention provides, in part, consensus antigens capable of eliciting an immune response in a subject against a prostate antigen. The consensus antigen can comprise epitopes that make them particularly effective as immunogens against prostate cancer cells can be induced. The consensus prostate antigen can comprise the full length translation product, a variant thereof, a fragment thereof or a combination thereof.

Twelve different consensus prostate antigens have been designed. Two of the consensus prostate antigens are consensus PAP antigen sequence 1 (SEQ ID NO: 2) and consensus PAP antigen sequence 2 lacking start and stop codon (SEQ ID NO: 4). Two of the consensus prostate antigens are consensus PARM1 antigen sequence 1 (SEQ ID NO: 6) and consensus PARM1 antigen sequence 2 lacking start and stop codon (SEQ ID NO: 8). Two of the consensus prostate antigens are consensus PCTA antigen sequence 1 (SEQ ID NO: 10) and consensus PCTA antigen sequence 2 lacking start and stop codon (SEQ ID NO: 12). Two of the consensus prostate antigens are consensus PSCA antigen sequence 1 (SEQ ID NO: 14) and consensus PSCA antigen sequence 2 lacking start and stop codon (SEQ ID NO: 16). Two of the consensus prostate antigens are consensus PSP94 antigen sequence 1 (SEQ ID NO: 18) and consensus PSP94 antigen sequence 2 lacking start and stop codon (SEQ ID NO: 20). Two of the consensus prostate antigens are consensus STEAP (also referred to herein as STEAP1) antigen sequence 1 (SEQ ID NO: 22) and consensus STEAP antigen sequence 2 lacking start and stop codon (SEQ ID NO: 24).

In some embodiments, the proteins may comprise sequences homologous to the prostate antigens, fragments of the prostate antigens, and/or proteins with sequences homologous to fragments of the prostate antigens.

In some embodiments, the proteins may comprise one or more sequences that are at least about 60%, at least about 70%, at least about 80%, or at least about 90% homologous to consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

For example, in some embodiments, the consensus proteins may comprise one or more sequences that are at least about 90% homologous to consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the consensus proteins may comprise one or more sequences that are at least about 95% homologous to consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the consensus proteins may comprise one or more sequences that are at least about 98% homologous to consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the consensus proteins may comprise one or more sequences that are at least about 99% homologous to consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

For example, in some embodiments, the consensus proteins may comprise one or more proteins selected from the group comprising: a) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof, b) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof, c) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof, d) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof, e) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof, f) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof, g) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof, h) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof, i) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof, j) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof, k) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and l) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

The consensus PAP antigen sequence is about 386 amino acids. Fragments of consensus PAP antigen 1 may comprise at least about 60%, 70%, 80%, 90%, 91%, 92%, 93%, 94%, 95%, 96%, 97%, 98%, or 99% of SEQ ID NO: 2. Fragments of consensus PAP antigen 2 lacking start and stop codon may comprise at least about 60%, 70%, 80%, 90%, 91%, 92%, 93%, 94%, 95%, 96%, 97%, 98%, or 99% of SEQ ID NO: 4. Fragments of consensus PAP antigen 1 may comprise 230, 255, 256, 257, 258, 259, 260, 280, 300, 345, 360, or 380 amino acids or more of SEQ ID NO: 2. Fragments of consensus PAP antigen 2 lacking start and stop codon may comprise 255, 256, 257, 258, 259, 260, 280, 300, 345, 360, or 380 amino acids or more of SEQ ID NO: 4.

The consensus PARM1 antigen sequence is about 310 amino acids. Fragments of consensus PARM1 antigen 1 may comprise at least about 60%, 70%, 80%, 90%, 91%, 92%, 93%, 94%, 95%, 96%, 97%, 98%, or 99% of SEQ ID NO: 4. Fragments of consensus PARM1 antigen 2 lacking start and stop codon may comprise at least about 60%, 70%, 80%, 90%, 91%, 92%, 93%, 94%, 95%, 96%, 97%, 98%, or 99% of SEQ ID NO: 8. Fragments of consensus PARM1 antigen 1 may comprise 230, 255, 256, 257, 258, 259, 260, 280, 290, 295, 300, or 304 amino acids or more of SEQ ID NO: 4. Fragments of consensus PARM1 antigen 2 lacking start and stop codon may comprise 255, 256, 257, 258, 259, 260, 280, 290, 295, 300, or 304 amino acids or more of SEQ ID NO: 8.

The consensus PCTA antigen sequence is about 316 amino acids. Fragments of consensus PCTA antigen 1 may comprise at least about 60%, 70%, 80%, 90%, 91%, 92%, 93%, 94%, 95%, 96%, 97%, 98%, or 99% of SEQ ID NO: 10. Fragments of consensus PCTA antigen 2 lacking start and stop codon may comprise at least about 60%, 70%, 80%, 90%, 91%, 92%, 93%, 94%, 95%, 96%, 97%, 98%, or 99% of SEQ ID NO: 12. Fragments of consensus PCTA antigen 1 may comprise 230, 255, 256, 257, 258, 259, 260, 280, 290, 295, 300, or 312 amino acids or more of SEQ ID NO: 10. Fragments of consensus PCTA antigen 2 lacking start and stop codon may comprise 255, 256, 257, 258, 259, 260, 280, 290, 295, 300, or 312 amino acids or more of SEQ ID NO: 12.

The consensus PSCA antigen sequence is about 123 amino acids. Fragments of consensus PSCA antigen 1 may comprise at least about 60%, 70%, 80%, 90%, 91%, 92%, 93%, 94%, 95%, 96%, 97%, 98%, or 99% of SEQ ID NO: 14. Fragments of consensus PSCA antigen 2 lacking start and stop codon may comprise at least about 60%, 70%, 80%, 90%, 91%, 92%, 93%, 94%, 95%, 96%, 97%, 98%, or 99% of SEQ ID NO: 16. Fragments of consensus PSCA antigen 1 may comprise 55, 56, 57, 58, 59, 60, 80, 100, 115, 116, or 120 amino acids or more of SEQ ID NO: 14. Fragments of consensus PSCA antigen 2 lacking start and stop codon may comprise 55, 56, 57, 58, 59, 60, 80, 100, 115, 116, or 120 amino acids or more of SEQ ID NO: 16.

The consensus PSP94 antigen sequence is about 114 amino acids. Fragments of consensus PSP94 antigen 1 may comprise at least about 60%, 70%, 80%, 90%, 91%, 92%, 93%, 94%, 95%, 96%, 97%, 98%, or 99% of SEQ ID NO: 18. Fragments of consensus PSP94 antigen 2 lacking start and stop codon may comprise at least about 60%, 70%, 80%, 90%, 91%, 92%, 93%, 94%, 95%, 96%, 97%, 98%, or 99% of SEQ ID NO: 20. Fragments of consensus PSP94 antigen 1 may comprise 55, 56, 57, 58, 59, 60, 80, 100, or 112 amino acids or more of SEQ ID NO: 18. Fragments of consensus PSP94 antigen 2 lacking start and stop codon may comprise 55, 56, 57, 58, 59, 60, 80, 100, or 112 amino acids or more of SEQ ID NO: 20.

The consensus STEAP1 antigen sequence is about 339 amino acids. Fragments of consensus STEAP1 antigen 1 may comprise at least about 60%, 70%, 80%, 90%, 91%, 92%, 93%, 94%, 95%, 96%, 97%, 98%, or 99% of SEQ ID NO: 22. Fragments of consensus STEAP1 antigen 2 lacking start and stop codon may comprise at least about 60%, 70%, 80%, 90%, 91%, 92%, 93%, 94%, 95%, 96%, 97%, 98%, or 99% of SEQ ID NO: 24. Fragments of consensus STEAP1 antigen 1 may comprise 230, 255, 256, 257, 258, 259, 260, 280, 300, 315, 320, or 330 amino acids or more of SEQ ID NO: 22. Fragments of consensus STEAP1 antigen 2 lacking start and stop codon may comprise 255, 256, 257, 258, 259, 260, 280, 300, 315, 320, or 330 amino acids or more of SEQ ID NO: 24.

In various embodiments, homology of multiple sequence alignments and phylogram were generated using ClustalW, a general purpose multiple sequence alignment program for DNA or proteins.

As noted above, in some embodiments, the consensus protein sequences may comprise a leader sequence at the N terminus. In some embodiments, the leader sequence is an IgE leader sequence as set forth in SEQ ID NO: 26. In some embodiments, the consensus protein sequences provided herein lack an IgE leader sequence as set forth in SEQ ID NO: 26. In some embodiments of the nucleic acid sequences provided herein, SEQ ID NO: 25 (which encodes SEQ ID NO: 26) is removed therefrom.

Accordingly, in some embodiments, the consensus proteins may comprise a signal peptide linked to SEQ ID NO: 2, SEQ ID NO: 6, SEQ ID NO: 10, SEQ ID NO: 14, SEQ ID NO: 18, or SEQ ID NO: 22, or a variant or fragment thereof in place of the N terminal methionine set forth in the claim (the coding sequence of the signal peptide typically includes a start codon encoding an N terminal methionine). Some embodiments relate to a protein that comprises a signal peptide linked to SEQ ID NO: 4, SEQ ID NO: 8, SEQ ID NO: 12, SEQ ID NO: 16, SEQ ID NO: 20, or SEQ ID NO: 24, or a variant or fragment thereof.

Thus, in some embodiments, the consensus proteins of the present invention comprises one or more proteins selected from the group comprising: a) an amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; b) an amino acid sequence having at least about 90% identity over an entire length of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; c) an immunogenic fragment comprising at least about 90% identity over at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; and/or d) an immunogenic fragment comprising at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence.

### Genetic Sequences, Constructs and Plasmids

The present invention provides, in part, nucleic acid molecules encoding the consensus amino acid sequences were generated to optimize stability and expression in humans. Codon selection was determined based upon, inter alia, an effort to minimize intramolecular interactions and secondary structure formation as well as using codons which result in improved expression. Vaccines may comprise one or more nucleic acid sequences that encode one or more of the consensus versions of the immunogenic proteins selected from this group of sequences generated to optimize stability and expression in humans. In some embodiments, the nucleic acid sequences incorporating coding sequence for the IgE leader at the 5′ end of the optimized, consensus encoding nucleic acid sequence were generated which encoded proteins having the IgE leader sequence (e.g., SEQ ID NO: 26) at the N terminus of the consensus amino acid sequence. In some embodiments, the nucleic acid sequence that encodes the IgE leader is SEQ ID NO: 25. In some embodiments, the nucleic acid sequences lack coding sequence for the IgE leader at the 5′ end of the optimized, consensus encoding nucleic acid sequence were generated which encoded proteins having the IgE leader sequence (e.g., SEQ ID NO: 26) at the N terminus of the consensus amino acid sequence.

Nucleic acid sequences are provided which encode consensus PAP antigen sequence 1 (protein sequence SEQ ID NO: 2; nucleic acid sequence SEQ ID NO: 1), consensus PAP antigen sequence 2 lacking start and stop codon (protein sequence SEQ ID NO: 4; nucleic acid sequence SEQ ID NO: 3), consensus PARM1 antigen sequence 1 (protein sequence SEQ ID NO: 6; nucleic acid sequence SEQ ID NO: 5), consensus PARM1 antigen sequence 2 lacking start and stop codon (protein sequence SEQ ID NO: 8; nucleic acid sequence SEQ ID NO: 7), consensus PCTA antigen sequence 1 (protein sequence SEQ ID NO: 10; nucleic acid sequence SEQ ID NO: 9), consensus PCTA antigen sequence 2 lacking start and stop codon (protein sequence SEQ ID NO: 12; nucleic acid sequence SEQ ID NO: 11), consensus PSCA antigen sequence 1 (protein sequence SEQ ID NO: 14; nucleic acid sequence SEQ ID NO: 13), consensus PSCA antigen sequence 2 lacking start and stop codon (protein sequence SEQ ID NO: 16), consensus PSP94 antigen sequence 1 (protein sequence SEQ ID NO: 18; nucleic acid sequence SEQ ID NO: 17), consensus PSP94 antigen sequence 2 lacking start and stop codon (protein sequence SEQ ID NO: 20; nucleic acid sequence SEQ ID NO: 19), consensus STEAP (also referred to herein as STEAP1) antigen sequence 1 (protein sequence SEQ ID NO: 22; nucleic acid sequence SEQ ID NO: 21), and/or consensus STEAP antigen sequence 2 lacking start and stop codon (protein sequence SEQ ID NO: 24; nucleic acid sequence SEQ ID NO: 23).

In some embodiments, the consensus PAP antigen is encoded by SEQ ID NO: 1 and comprises a proteins having an amino acid sequence of SEQ ID NO: 2. In some embodiments, the consensus PAP antigen is encoded by SEQ ID NO: 3 and comprises a proteins having an amino acid sequence of SEQ ID NO: 4. In some embodiments, the consensus PARM1 antigen is encoded by SEQ ID NO: 5 and comprises a proteins having an amino acid sequence of SEQ ID NO: 6. In some embodiments, the consensus PARM1 antigen is encoded by SEQ ID NO: 7 and comprises a proteins having an amino acid sequence of SEQ ID NO: 8. In some embodiments, the consensus PCTA antigen is encoded by SEQ ID NO: 9 and comprises a proteins having an amino acid sequence of SEQ ID NO: 10. In some embodiments, the consensus PCTA antigen is encoded by SEQ ID NO: 11 and comprises a proteins having an amino acid sequence of SEQ ID NO: 12. In some embodiments, the consensus PSCA antigen is encoded by SEQ ID NO: 13 and comprises a proteins having an amino acid sequence of SEQ ID NO: 14. In some embodiments, the consensus PSCA antigen is encoded by SEQ ID NO: 15 and comprises a proteins having an amino acid sequence of SEQ ID NO: 16. In some embodiments, the consensus PSP94 antigen is encoded by SEQ ID NO: 17 and comprises a proteins having an amino acid sequence of SEQ ID NO: 18. In some embodiments, the consensus PSP94 antigen is encoded by SEQ ID NO: 19 and comprises a proteins having an amino acid sequence of SEQ ID NO: 20. In some embodiments, the consensus STEAP1 antigen is encoded by SEQ ID NO: 21 and comprises a proteins having an amino acid sequence of SEQ ID NO: 22. In some embodiments, the consensus STEAP1 antigen is encoded by SEQ ID NO: 23 and comprises a proteins having an amino acid sequence of SEQ ID NO: 24.

Consensus PAP antigen is encoded by nucleotides 1-1164 of SEQ ID NO: 1 and comprises a proteins having an amino acid sequence of SEQ ID NO: 2. The coding sequence having nucleotides 1-1164 of SEQ ID NO: 1 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus PAP antigen sequence 1 (SEQ ID NO: 2).

Consensus PAP antigen is encoded by nucleotides 1-1155 of SEQ ID NO: 3 and comprises a proteins having an amino acid sequence of SEQ ID NO: 4. Isolated nucleic acid molecules can encode proteins that have sequences at least about 60%, 70%, 80%, 90%, 91%, 92%, 93%, 94%, 95%, 96%, 97%, 98%, or 99% homologous to consensus PAP antigen sequence 2 lacking start and stop codon (SEQ ID NO: 4).

Consensus PARM1 antigen is encoded by nucleotides 1-936 of SEQ ID NO: 5 and comprises a proteins having an amino acid sequence of SEQ ID NO: 6. The coding sequence having nucleotides 1-936 of SEQ ID NO: 5 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus PARM1 antigen sequence 1 (SEQ ID NO: 6).

Consensus PARM1 antigen is encoded by nucleotides 1-927 of SEQ ID NO: 7 and comprises a proteins having an amino acid sequence of SEQ ID NO: 8. Isolated nucleic acid molecules can encode proteins that have sequences at least about 60%, 70%, 80%, 90%, 91%, 92%, 93%, 94%, 95%, 96%, 97%, 98%, or 99% homologous to consensus PARM1 antigen sequence 2 lacking start and stop codon (SEQ ID NO: 8).

Consensus PCTA antigen is encoded by nucleotides 1-954 of SEQ ID NO: 9 and comprises a proteins having an amino acid sequence of SEQ ID NO: 10. The coding sequence having nucleotides 1-954 of SEQ ID NO: 9 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus PCTA antigen sequence 1 (SEQ ID NO: 10).

Consensus PCTA antigen is encoded by nucleotides 1-945 of SEQ ID NO: 11 and comprises a proteins having an amino acid sequence of SEQ ID NO: 12. Isolated nucleic acid molecules can encode proteins that have sequences at least about 60%, 70%, 80%, 90%, 91%, 92%, 93%, 94%, 95%, 96%, 97%, 98%, or 99% homologous to consensus PCTA antigen sequence 2 lacking start and stop codon (SEQ ID NO: 12).

Consensus PSCA antigen is encoded by nucleotides 1-375 of SEQ ID NO: 13 and comprises a proteins having an amino acid sequence of SEQ ID NO: 14. The coding sequence having nucleotides 1-375 of SEQ ID NO: 13 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus PSCA antigen sequence 1 (SEQ ID NO: 14).

Consensus PSCA antigen is encoded by nucleotides 1-366 of SEQ ID NO: 15 and comprises a proteins having an amino acid sequence of SEQ ID NO: 16. Isolated nucleic acid molecules can encode proteins that have sequences at least about 60%, 70%, 80%, 90%, 91%, 92%, 93%, 94%, 95%, 96%, 97%, 98%, or 99% homologous to consensus PSCA antigen sequence 2 lacking start and stop codon (SEQ ID NO: 16).

Consensus PSP94 antigen is encoded by nucleotides 1-348 of SEQ ID NO: 17 and comprises a proteins having an amino acid sequence of SEQ ID NO: 18. The coding sequence having nucleotides 1-348 of SEQ ID NO: 17 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus PSP94 antigen sequence 1 (SEQ ID NO: 18).

Consensus PSP94 antigen is encoded by nucleotides 1-339 of SEQ ID NO: 19 and comprises a proteins having an amino acid sequence of SEQ ID NO: 20. Isolated nucleic acid molecules can encode proteins that have sequences at least about 60%, 70%, 80%, 90%, 91%, 92%, 93%, 94%, 95%, 96%, 97%, 98%, or 99% homologous to consensus PSP94 antigen sequence 2 lacking start and stop codon (SEQ ID NO: 20).

Consensus STEAP1 antigen is encoded by nucleotides 1-1023 of SEQ ID NO: 21 and comprises a proteins having an amino acid sequence of SEQ ID NO: 22. The coding sequence having nucleotides 1-1023 of SEQ ID NO: 21 has one or more stop codons at its 3′ end. Isolated nucleic acid molecules can encode proteins that have sequences at least about 60%, 70%, 80%, 90%, 91%, 92%, 93%, 94%, 95%, 96%, 97%, 98%, or 99% homologous to consensus STEAP1 antigen sequence 1 (SEQ ID NO: 22).

Consensus STEAP1 antigen is encoded by nucleotides 1-1014 of SEQ ID NO: 23 and comprises a proteins having an amino acid sequence of SEQ ID NO: 24. Isolated nucleic acid molecules can encode proteins that have sequences at least about 60%, 70%, 80%, 90%, 91%, 92%, 93%, 94%, 95%, 96%, 97%, 98%, or 99% homologous to consensus STEAP1 antigen sequence 2 lacking start and stop codon (SEQ ID NO: 24).

Isolated nucleic acid molecules can encode proteins that have sequences at least about 60%, 70%, 80%, 90%, 91%, 92%, 93%, 94%, 95%, 96%, 97%, 98%, or 99% of consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

For example, in some embodiments, the isolated nucleic acid molecules can encode proteins that have sequences at least about 90% of consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the isolated nucleic acid molecules can encode proteins that have sequences at least about 95% of consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the isolated nucleic acid molecules can encode proteins that have sequences at least about 98% of consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the isolated nucleic acid molecules can encode proteins that have sequences at least about 99% of consensus PAP antigen sequence 1 (SEQ ID NO: 2), consensus PAP antigen sequence 2 lacking start and stop codon (SEQ ID NO: 4), consensus PARM1 antigen sequence 1 (SEQ ID NO: 6), consensus PARM1 antigen sequence 2 lacking start and stop codon (SEQ ID NO: 8), consensus PCTA antigen sequence 1 (SEQ ID NO: 10), consensus PCTA antigen sequence 2 lacking start and stop codon (SEQ ID NO: 12), consensus PSCA antigen sequence 1 (SEQ ID NO: 14), consensus PSCA antigen sequence 2 lacking start and stop codon (SEQ ID NO: 16), consensus PSP94 antigen sequence 1 (SEQ ID NO: 18), consensus PSP94 antigen sequence 2 lacking start and stop codon (SEQ ID NO: 20), consensus STEAP (also referred to herein as STEAP1) antigen sequence 1 (SEQ ID NO: 22), and/or consensus STEAP antigen sequence 2 lacking start and stop codon (SEQ ID NO: 24).

In some embodiments, the isolated nucleic acid molecules can encode proteins that comprise a leader sequence at the N terminus. In some embodiments, the isolated nucleic acid molecules can encode proteins that lack a leader sequence at the N terminus. In some embodiments, the nucleic acid molecules can encode the IgE leader sequence that is SEQ ID NO: 26. Thus, in some embodiments of the nucleic acid sequences provided herein, SEQ ID NO: 25 (which encodes SEQ ID NO: 26) is removed therefrom.

In some embodiments, the isolated nucleic acid molecules can encode proteins that comprise a signal peptide linked to SEQ ID NO: 4, SEQ ID NO: 8, SEQ ID NO: 12, SEQ ID NO: 16, SEQ ID NO: 20, or SEQ ID NO: 24, or a variant or fragment thereof. In some embodiments, the isolated nucleic acid molecules can encode proteins that comprise a signal peptide linked to SEQ ID NO: 2, SEQ ID NO: 6, SEQ ID NO: 10, SEQ ID NO: 14, SEQ ID NO: 18, or SEQ ID NO: 22, or a variant or fragment thereof in place of the N terminal methionine set forth in the claim (the coding sequence of the signal peptide typically includes a start codon encoding an N terminal methionine).

In some embodiments, the nucleic acid molecules comprise a coding sequence encoding one or more proteins selected from the group comprising: a) an amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; b) an amino acid sequence having at least about 90% identity over an entire length of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; c) an immunogenic fragment comprising at least about 90% identity over at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence; and/or d) an immunogenic fragment comprising at least 60% of the amino acid sequence selected from the group consisting of SEQ ID NO:2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, or any combination thereof linked to an IgE signal peptide sequence (SEQ ID NO: 26) or a signal sequence other than the IgE signal peptide sequence.

In one aspect, the present invention relates, in part, to nucleic acid molecules comprising nucleic acid coding sequences that have been generated to improve and optimize expression. The codons used in these nucleic acid molecules were selected to generate RNA having reduced secondary structure formation due to intramolecular hybridization. Nucleic acid sequences encoding PAP consensus antigen, PARM11 consensus antigen, PCTA consensus antigen, PSCA consensus antigen, PSP94 consensus antigen, STEAP consensus antigen, or any combination thereof, are disclosed. Two consensus nucleotide sequences for PAP are disclosed: PAP Consensus Antigen sequence 1 (SEQ ID NO: 1) and PAP Consensus Antigen sequence 2 lacking start and stop codon (SEQ ID NO: 3). Two consensus nucleotide sequences for PARM1 are disclosed: PARM1 Consensus Antigen sequence 1 (SEQ ID NO: 5) and PARM1 Consensus Antigen sequence 2 lacking start and stop codon (SEQ ID NO: 7). Two consensus nucleotide sequences for PCTA are disclosed: PCTA Consensus Antigen sequence 1 (SEQ ID NO: 9) and PCTA Consensus Antigen sequence 2 lacking start and stop codon (SEQ ID NO: 11). Two consensus nucleotide sequences for PSCA are disclosed: PSCA Consensus Antigen sequence 1 (SEQ ID NO: 13) and PSCA Consensus Antigen sequence 2 lacking start and stop codon (SEQ ID NO: 15). Two consensus nucleotide sequences for PSP94 are disclosed: PSP94 Consensus Antigen sequence 1 (SEQ ID NO: 17) and PSP94 Consensus Antigen sequence 2 lacking start and stop codon (SEQ ID NO: 19). Two consensus nucleotide sequences for STEAP (also referred to herein as STEAP1) are disclosed: STEAP Consensus Antigen sequence 1 (SEQ ID NO: 21) and STEAP Consensus Antigen sequence 2 lacking start and stop codon (SEQ ID NO: 23).

Provided herein are genetic constructs that can comprise a nucleic acid sequence that encodes consensus prostate antigen disclosed herein including consensus protein sequences, sequences homologous to consensus protein sequences, fragments of consensus protein sequences and sequences homologous to fragments of consensus protein sequences. The genetic construct can be present in the cell as a functioning extrachromosomal molecule. The genetic construct can be linear minichromosome including centromere, telomers or plasmids or cosmids.

The genetic construct can also be part of a genome of a recombinant viral vector, including recombinant adenovirus, recombinant adenovirus associated virus and recombinant vaccinia. The genetic construct can be part of the genetic material in attenuated live microorganisms or recombinant microbial vectors which live in cells. The genetic constructs can comprise regulatory elements for gene expression of the coding sequences of the nucleic acid. The regulatory elements can be a promoter, an enhancer an initiation codon, a stop codon, or a polyadenylation signal.

The nucleic acid sequences may make up a genetic construct that can be a vector. The vector can be capable of expressing an antigen in the cell of a mammal in a quantity effective to elicit an immune response in the mammal. The vector can be recombinant. The vector can comprise heterologous nucleic acid encoding the antigen. The vector can be a plasmid. The vector can be useful for transfecting cells with nucleic acid encoding an antigen, which the transformed host cell is cultured and maintained under conditions wherein expression of the antigen takes place.

In some embodiments, coding sequences for a single consensus prostate antigen is provided on a single vector. In some embodiments, coding sequences for a multiple consensus prostate antigen are provided on a single vector. In some embodiments, compositions are provided comprising coding sequences for a multiple consensus prostate antigens on multiple vectors, either one antigen per vector or multiple antigens per vector.

In some embodiments, coding sequences for two or more different consensus prostate antigens may be provided on a single vector. In some embodiments, the coding sequences may have separate promoters controlling expression. In some embodiments, the coding sequences may have a single promoters controlling expression with an IRES sequence separating coding sequence. The presence of the IRES sequence results in the separate translation of the transcription product. In some embodiments, the coding sequences may have a single promoters controlling expression with coding sequence encoding a proteolytic cleavage peptide sequence separating coding sequences of the antigens. A single translation product is produced which is then processed by the protease that recognizes the protease cleavage site to generate separate protein molecules. The protease cleave sites used is typically recognized by a protease endogenously present in the cell where expression occurs. In some embodiments, a separate coding sequence for a protease may be included to provide for the production of the protease needed to process the polyprotein translation product. In some embodiment, vectors comprise coding sequences for one, two, three, four, five, six or all seven consensus prostate antigens.

In each and every instance set forth herein, coding sequences may be optimized for stability and high levels of expression. In some instances, codons are selected to reduce secondary structure formation of the RNA such as that formed due to intramolecular bonding.

The vector can comprise heterologous nucleic acid encoding an antigen and can further comprise an initiation codon, which can be upstream of the antigen coding sequence, and a stop codon, which can be downstream of the antigen coding sequence. The initiation and termination codon can be in frame with the antigen coding sequence. The vector can also comprise a promoter that is operably linked to the antigen coding sequence. The promoter operably linked to the antigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the consensus prostate antigen coding sequence. The polyadenylation signal can be a SV40 polyadenylation signal, LTR polyadenylation signal, bovine growth hormone (bGH) polyadenylation signal, human growth hormone (hGH) polyadenylation signal, or human (3-globin polyadenylation signal. The SV40 polyadenylation signal can be a polyadenylation signal from a pCEP4 vector (Invitrogen, San Diego, CA).

The vector can also comprise an enhancer upstream of the consensus prostate antigen coding sequence. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference.

The vector can also comprise a mammalian origin of replication in order to maintain the vector extrachromosomally and produce multiple copies of the vector in a cell. The vector can be pVAXl, pCEP4 or pREP4 from Invitrogen (San Diego, CA), which can comprise the Epstein Barr virus origin of replication and nuclear antigen EBNA-1 coding region, which can produce high copy episomal replication without integration. The backbone of the vector can be pAV0242. The vector can be a replication defective adenovirus type 5 (Ad5) vector.

The vector can also comprise a regulatory sequence, which can be well suited for gene expression in a mammalian or human cell into which the vector is administered. The consensus prostate antigen coding sequence can comprise a codon, which can allow more efficient transcription of the coding sequence in the host cell.

The vector can be pSE420 (Invitrogen, San Diego, Calif), which can be used for protein production in Escherichia coli (E. coli). The vector can also be pYES2 (Invitrogen, San Diego, Calif), which can be used for protein production in Saccharomyces cerevisiae strains of yeast. The vector can also be of the MAXBAC™ complete baculovirus expression system (Invitrogen, San Diego, Calif), which can be used for protein production in insect cells. The vector can also be pcDNA I or pcDNA3 (Invitrogen, San Diego, Calif), which may be used for protein production in mammalian cells such as Chinese hamster ovary (CHO) cells. The vector can be expression vectors or systems to produce protein by routine techniques and readily available starting materials including Sambrook et al, Molecular Cloning and Laboratory Manual, Second Ed., Cold Spring Harbor (1989), which is incorporated fully by reference.

### Vaccines

Vaccines may comprise one or more of the prostate antigens set forth herein and/or vaccines may comprise one or more nucleic acid sequences that encode one or more of the consensus prostate antigen selected from this group. Vaccines may comprise one or more of the consensus prostate antigens set forth herein in combination with other immunogenic prostate proteins with sequences other than the consensus sequences disclosed herein including native sequences and/or vaccines may comprise one or more nucleic acid sequences that encode one or more of the consensus prostate antigens selected from this group in combination with nucleic acid molecules that encode other prostate antigens with sequences other than the consensus sequences disclosed herein.

In various embodiments, the vaccine of the present invention that can be used to elicit an immune response (humoral, cellular, or both) broadly against prostate cancer cells may comprise one or more of the following nucleic acid sequences that encodes one or more proteins selected from consensus PAP antigen sequence 1, consensus PAP antigen sequence 2 lacking start and stop codon, consensus PARM1 antigen sequence 1, consensus PARM11 antigen sequence 2 lacking start and stop codon, consensus PCTA antigen sequence 1, consensus PCTA antigen sequence 2 lacking start and stop codon, consensus PSCA antigen sequence 1, consensus PSCA antigen sequence 2 lacking start and stop codon, consensus PSP94 antigen sequence 1 (SEQ ID NO: 18), consensus PSP94 antigen sequence 2 lacking start and stop codon, consensus STEAP (also referred to herein as STEAP1) antigen sequence 1, consensus STEAP antigen sequence 2 lacking start and stop codon, or any combination thereof. Coding sequences may also include those provided herein that comprise homologous sequences, fragments, and homologous sequences of fragments.

Some embodiments provide methods of generating immune responses against prostate cancer cells comprise administering to a subject one or more compositions which collectively comprise one or more coding sequences or combinations described herein. Some embodiments provide methods of prophylactically vaccinating a subject against prostate cancer comprise administering one or more compositions which collectively comprise one or more coding sequences or combinations described herein. Some embodiments provide methods of therapeutically vaccinating a subject has prostate cancer that comprise administering one or more compositions which collectively comprise one or more coding sequences or combinations described herein.

### Pharmaceutical Compositions

Provided herein are pharmaceutical compositions according to the present invention which comprise about 1 nanogram to about 10 mg of DNA. In some embodiments, pharmaceutical compositions according to the present invention comprise from between: 1) at least 10, 15, 20, 25, 30, 35, 40, 45, 50, 55, 60, 65, 70, 75, 80, 85, 90, 95 or 100 nanograms, or at least 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995 or 1000 micrograms, or at least 1.5, 2, 2.5, 3, 3.5, 4, 4.5, 5, 5.5, 6, 6.5, 7, 7.5, 8, 8.5, 9, 9.5 or 10 mg or more; and 2) up to and including 15, 20, 25, 30, 35, 40, 45, 50, 55, 60, 65, 70, 75, 80, 85, 90, 95 or 100 nanograms, or up to and including 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895. 900, 905, 910, 915, 920, 925, 930, 935, 940, 945, 950, 955, 960, 965, 970, 975, 980, 985, 990, 995, or 1000 micrograms, or up to and including 1.5, 2, 2.5, 3, 3.5, 4, 4.5, 5, 5.5, 6, 6.5, 7, 7.5, 8, 8.5, 9, 9.5 or 10 mg. In some embodiments, pharmaceutical compositions according to the present invention comprise about 5 nanogram to about 10 mg of DNA. In some embodiments, pharmaceutical compositions according to the present invention comprise about 25 nanogram to about 5 mg of DNA. In some embodiments, the pharmaceutical compositions contain about 50 nanograms to about 1 mg of DNA. In some embodiments, the pharmaceutical compositions contain about 0.1 to about 500 micrograms of DNA. In some embodiments, the pharmaceutical compositions contain about 1 to about 350 micrograms of DNA. In some embodiments, the pharmaceutical compositions contain about 5 to about 250 micrograms of DNA. In some embodiments, the pharmaceutical compositions contain about 10 to about 200 micrograms of DNA. In some embodiments, the pharmaceutical compositions contain about 15 to about 150 micrograms of DNA. In some embodiments, the pharmaceutical compositions contain about 20 to about 100 micrograms of DNA. In some embodiments, the pharmaceutical compositions contain about 25 to about 75 micrograms of DNA. In some embodiments, the pharmaceutical compositions contain about 30 to about 50 micrograms of DNA. In some embodiments, the pharmaceutical compositions contain about 35 to about 40 micrograms of DNA. In some embodiments, the pharmaceutical compositions contain about 100 to about 200 microgram DNA. In some embodiments, the pharmaceutical compositions comprise about 10 microgram to about 100 micrograms of DNA. In some embodiments, the pharmaceutical compositions comprise about 20 micrograms to about 80 micrograms of DNA. In some embodiments, the pharmaceutical compositions comprise about 25 micrograms to about 60 micrograms of DNA. In some embodiments, the pharmaceutical compositions comprise about 30 nanograms to about 50 micrograms of DNA. In some embodiments, the pharmaceutical compositions comprise about 35 nanograms to about 45 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

Preferably the pharmaceutical composition is a vaccine, and more preferably a DNA vaccine.

The vaccine may be a DNA vaccine. The DNA vaccine may comprise a plurality of the same or different plasmids comprising nucleic acid coding sequences for one or more of consensus prostate antigens. The DNA vaccine may comprise one or more nucleic acid sequences that encode one or more of consensus prostate antigens. When the DNA vaccine comprises coding sequences of more than one consensus prostate antigens all such sequences may be present on a single plasmid, or each such sequences may be present on a different plasmids.

In some embodiments, vaccines may comprise nucleic acid sequences that encode one or more of consensus prostate antigens in combination with one or more of consensus prostate antigens.

DNA vaccines are disclosed in U.S. Pat. Nos. 5,593,972, 5,739,118, 5,817,637, 5,830,876, 5,962,428, 5,981,505, 5,580,859, 5,703,055, and 5,676,594, which are incorporated herein fully by reference. The DNA vaccine can further comprise elements or reagents that inhibit it from integrating into the chromosome. The vaccine can be an RNA of the prostate antigen. The RNA vaccine can be introduced into the cell.

The vaccine can be a recombinant vaccine comprising the genetic construct or antigen described above. The vaccine can also comprise one or more consensus prostate antigens in the form of one or more protein subunits, or one or more attenuated viral particles comprising one or more consensus prostate antigens. The attenuated vaccine can be attenuated live vaccines, killed vaccines and vaccines that use recombinant vectors to deliver foreign genes that encode one or more consensus prostate antigens, and well as subunit and glycoprotein vaccines. Examples of attenuated live vaccines, those using recombinant vectors to deliver prostate antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

The vaccine provided may be used to induce immune responses including therapeutic or prophylactic immune responses. Antibodies and/or killer T cells may be generated which are directed to the consensus prostate antigen. Such antibodies and cells may be isolated.

The vaccine can further comprise a pharmaceutically acceptable excipient. The pharmaceutically acceptable excipient can be functional molecules as vehicles, adjuvants, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the vaccine at a concentration less than 6 mg/ml. The transfection facilitating agent can also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid can also be used administered in conjunction with the genetic construct. In some embodiments, the DNA vector vaccines can also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The pharmaceutically acceptable excipient may be an adjuvant. The adjuvant may be other genes that are expressed in alternative plasmid or are delivered as proteins in combination with the plasmid above in the vaccine. The adjuvant may be selected from the group consisting of: a-interferon (IFN-a), β-interferon (IFN-β), γ-interferon, platelet derived growth factor (PDGF), TNFa, TNF, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant may be IL-12, IL-15, IL-28, CTACK, TECK, platelet derived growth factor (PDGF), TNFa, TNFp, GM-CSF, epidermal growth factor (EGF), IL-1, IL-2, IL-4, IL-5, IL-6, IL-10, IL-12, IL-18, or a combination thereof.

Other genes which may be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

The vaccine can further comprise a genetic vaccine facilitator agent as described in U.S. Ser. No. 021,579 filed Apr. 1, 1994, which is fully incorporated by reference.

### Methods of Delivery

Provided herein is a method for delivering the pharmaceutical formulations, preferably vaccines, for providing genetic constructs and consensus prostate antigen which comprise epitopes that make them particular effective immunogens against which an immune response to prostate cancer cells can be induced. The method of delivering the vaccine, or vaccination, can be provided to induce a therapeutic and/or prophylactic immune response. The vaccine can be delivered to a subject to modulate the activity of the mammal's immune system and enhance the immune response.

Upon delivery of the vaccine to the mammal, and thereupon the vector into the cells of the mammal, the transfected cells will express and secrete the corresponding prostate consensus protein. These secreted proteins, or synthetic antigens, will be recognized by the immune system, which will mount an immune response that can include: antibodies made against the antigens, and T-cell response specifically against the antigen. In some examples, a mammal vaccinated with the vaccines discussed herein will have a primed immune system. The vaccine can be delivered to a subject to modulate the activity of the subject's immune system thereby enhancing the immune response.

The vaccine can be delivered in the form of a DNA vaccine and methods of delivering a DNA vaccines are described in U.S. Pat. Nos. 4,945,050 and 5,036,006, which are both incorporated fully by reference.

The vaccine can be administered to a mammal to elicit an immune response in a mammal. The mammal can be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

### Combination Treatments

The pharmaceutical compositions, preferably vaccines, can be administered in combination with one or more other prostate proteins or genes. The vaccine can be administered in combination with proteins or genes encoding adjuvants, which can include: a-interferon (IFN-a), β-interferon (IFN-β), γ-interferon, IL-12, IL-15, IL-28, CTACK, TECK, platelet derived growth factor (PDGF), TNFa, TNFp, GM-CSF, epidermal growth factor (EGF), IL-1, IL-2, IL-4, IL-5, IL-6, IL-10, IL-12, IL-18, MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PEC AM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, INK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, or TAP2, or functional fragments thereof.

### Routes of Administration

The vaccine can be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition can be administered as a suitably acceptable formulation in accordance with normal veterinary practice. The veterinarian can readily determine the dosing regimen and route of administration that is most appropriate for a particular animal. The vaccine can be administered by traditional syringes, needleless injection devices, “microprojectile bombardment gone guns”, or other physical methods such as electroporation (“EP”), “hydrodynamic method”, or ultrasound.

The vector of the vaccine can be delivered to the mammal by several well known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The prostate antigen can be delivered via DNA injection and along with in vivo electroporation.

### Electroporation

Administration of the vaccine via electroporation of the plasmids of the vaccine may be accomplished using electroporation devices that can be configured to deliver to a desired tissue of a mammal a pulse of energy effective to cause reversible pores to form in cell membranes, and in some embodiments, the pulse of energy is a constant current similar to a preset current input by a user.

In some embodiments where electroporation is utilized, the electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA® EP system (Inovio Pharmaceuticals, Inc., Blue Bell, PA) or Elgen electroporator (Inovio Pharmaceuticals, Inc., Blue Bell, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current. A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μL, 20 μL, 10 μL, or 1 μL, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the DNA vaccines of the present invention, include those described in U.S. Pat. No. 7,245,963 by Draghia-Akli, et al, U.S. Patent Pub. 2005/0052630 submitted by Smith, et al, the contents of which are hereby incorporated by reference in their entirety. Other electroporation devices and electroporation methods that may be used for facilitating delivery of the DNA vaccines include those provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety. U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety. Another embodiment of an electroporation device to be used with the cancer antigens described herein is the Elgen EP device (Inovio Pharmaceuticals, Inc., Blue Bell, PA).

### Method of Preparing Vaccine

Provided herein are methods for preparing the DNA plasmids that comprise the DNA vaccines discussed herein. The DNA plasmids, after the final subcloning step into the mammalian expression plasmid, can be used to inoculate a cell culture in a large scale fermentation tank, using known methods in the art.

The DNA plasmids for use with the EP devices of the present invention can be formulated or manufactured using a combination of known devices and techniques, but preferably they are manufactured using an optimized plasmid manufacturing technique that is described in a licensed, co-pending U.S. provisional application U.S. Ser. No. 60/939,792, which was filed on May 23, 2007. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Immunotherapy of Prostate Cancer Using Novel Synthetic DNA Vaccines Targeting Multiple Tumor Antigens

The prostate cancer full length antigens investigated in detail have focused on prostate-specific membrane antigen (PSMA), prostate serum antigen (PSA), and prostatic acid phosphatase (PAP) expressed through viral vectors, DNA vaccines, and personalized peptide vaccines (Zahm C D et al., 2017, Pharmacol Ther., 174:27-42; Boettcher A N et al., 2019, Front Oncol., 9:884). Cell therapies, such as chimeric antigen receptor (CAR)-T cells against PSMA, was also studied (Grullich C et al., 2020, Urologe A., 59:687-694). Different checkpoint inhibitors, bispecific antibodies, and oncolytic viruses have also been investigated as immunotherapeutics for PCa (Grullich C et al., 2020, Urologe A., 59:687-694). In addition, vaccination with messenger RNA (mRNA) encoding distinctive tumor antigens, which activated CD4 and CD8 cells, was also investigated in PCa. For instance, Curevac introduced RNActive, containing both free and protamine-bound mRNA directed to four different antigens, such as PSA, PSMA, PSCA and STEAP1, which demonstrated an immunological response through activation of B and T cells for all those antigens (Grullich C et al., 2020, Urologe A., 59:687-694).

Importantly, vast majority of the studies with these antigens have been carried out using single antigen. Considering the heterogeneous nature of prostate tumors, single antigen approach did not yield optimal immunity against cancer. Thus, in the present study, a Synthetic Enhanced DNA vaccine (SEV) platform was utilized to target multiple prostate cancer antigens. Both bioinformatic approaches and literature knowledge were utilized to select the SEV candidates. These included six-transmembrane epithelial antigen of the prostate-1 (STEAP1), prostatic acid phosphatase (PAP), prostate androgen regulated mucin-like protein 1 (PARM1), prostate carcinoma tumor antigen-1 (PCTA), prostate stem cell antigen (PSCA), and prostate secretory protein of 94 amino acids (PSP94). The present study has developed a synthetic consensus strategy where gene sequences from various species were compared to determine a consensus sequence exhibiting significant homology. Notably, this approach was found to break tolerance capacity, while retaining T cell killing against native major histocompatibility complex (MHC) class I-presented sequences (Duperret E K et al., 2018, Mol Ther., 26:435-445). All these six human genes presented in this example (i.e., STAEP1, PAP, PARM1, PSCA, PCTA, and PSP94) shared high homology with mouse; in fact, above 60% identity as revealed by Homologene, National Center for Biotechnology Information (NCBI) data base. Immune responses as well as demonstrated the anti-tumor activity of these novel therapeutics in prostate specific tumor challenge model were evaluated. The results showed that targeting multiple prostate cancer antigens was an effective strategy against prostate cancer and SEV was an attractive immunotherapeutic approach against cancer owing to its safety, simplicity, and stability (Duperret E K et al., 2018, Cancer Res., 78:6363-6370; Perales-Puchalt A et al., 2019, JCI Insight, 4; Tebas P et al., 2017, N Engl J Med.; Muthumani K et al., 2015, Sci Transl Med., 7:301ra132; Bagarazzi M L et al., 2012, Transl Med., 4:155ra38).

Generation and Characterization of PCaA-SEV

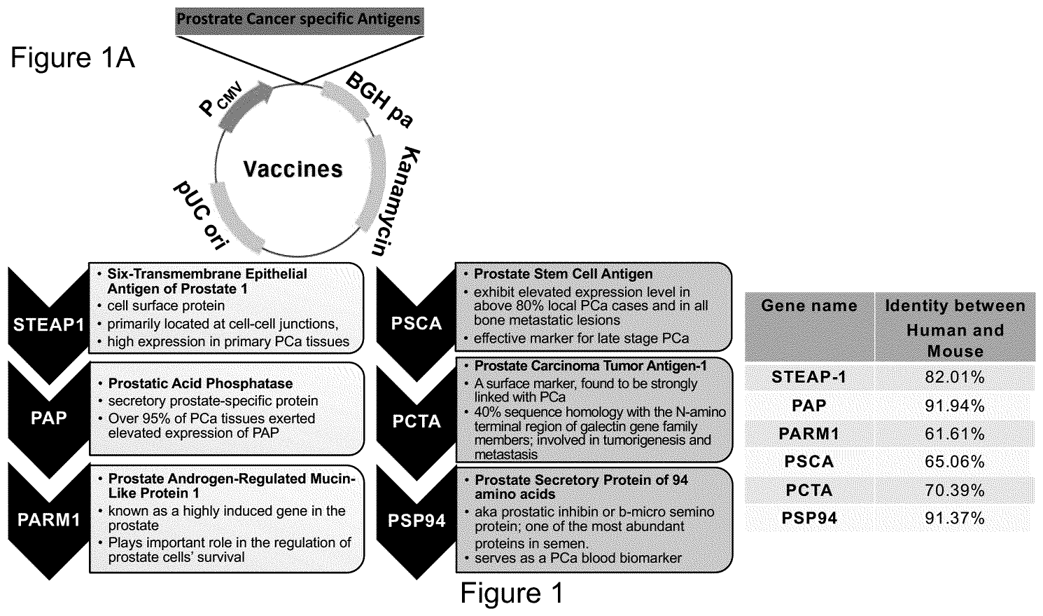

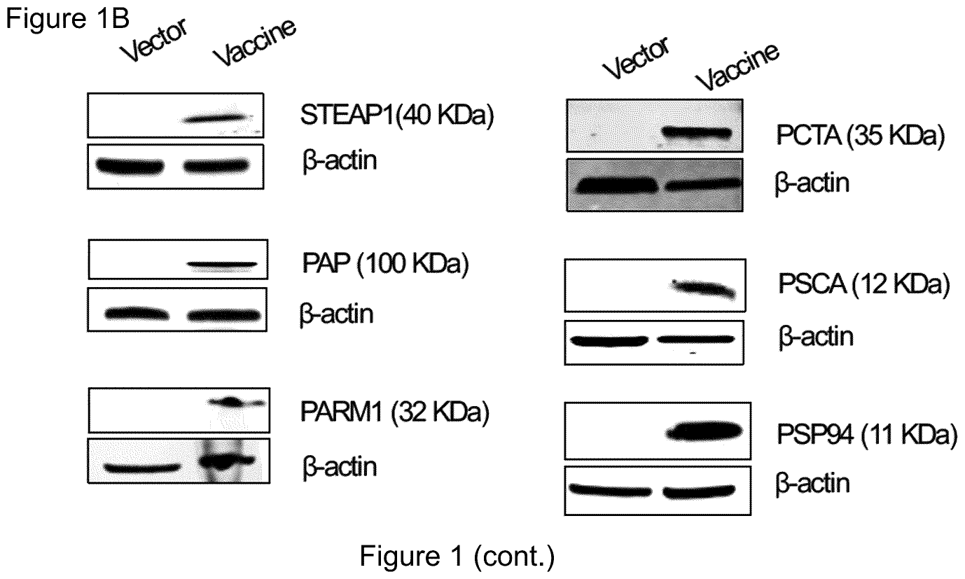

The selection of candidate tumor antigens was based on the information available from the published papers as well as the analysis of the collected databases on prostate cancer. The criteria that were used include overexpression of a gene in prostate tumors in comparison to normal prostate tissues or cells. In addition, overexpression of genes in relation to the early vs. advanced stage of disease was also taken into account. These efforts led to select STEAP1, PAP, PARM1, PSCA, PCTA, and PSP94 for the present studies. Synthetic full-length gene sequences of STEAP1, PAP, PARM1, PSCA, PCTA, and PSP94 were generated (FIG. 1A) and cloned successfully using bioinformatics and synthetic DNA technologies as described earlier (Duperret E K et al., 2018, Cancer Res., 78:6363-6370; Perales-Puchalt A et al., 2019, JCI Insight, 4; Muthumani K et al., 2017, Cancer Immunol Immunother., 66:1577-1588). For validating the prostate-specific proteins as target antigens for vaccination, initially studies focused on the evaluation of their expression by Western blot analysis using the lysates of cells transfected with PCaA-SEV. The results showed that lysates from human embryonic kidney 293 (HEK293T) cells transfected with PCaA-SEV revealed the correct molecular sizes corresponding to the expression of each PCaA protein. Expression of 3-actin served as endogenous control (FIG. 1i). Together, successful generation of six different PCaA-SEV and protein expression in cells was confirmed to proceed with further studies.

Effect of PCaA-SEV Immunization on Antigen Specific Cellular Immune Responses

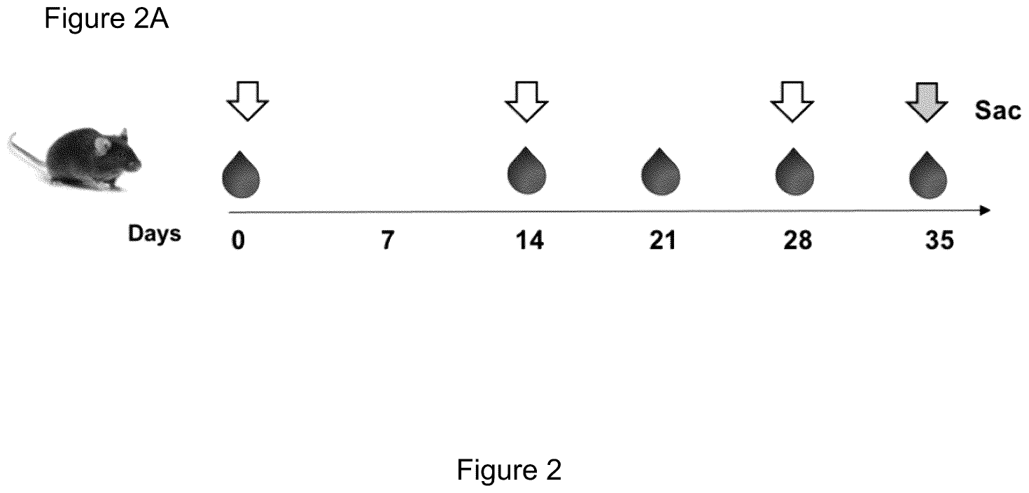

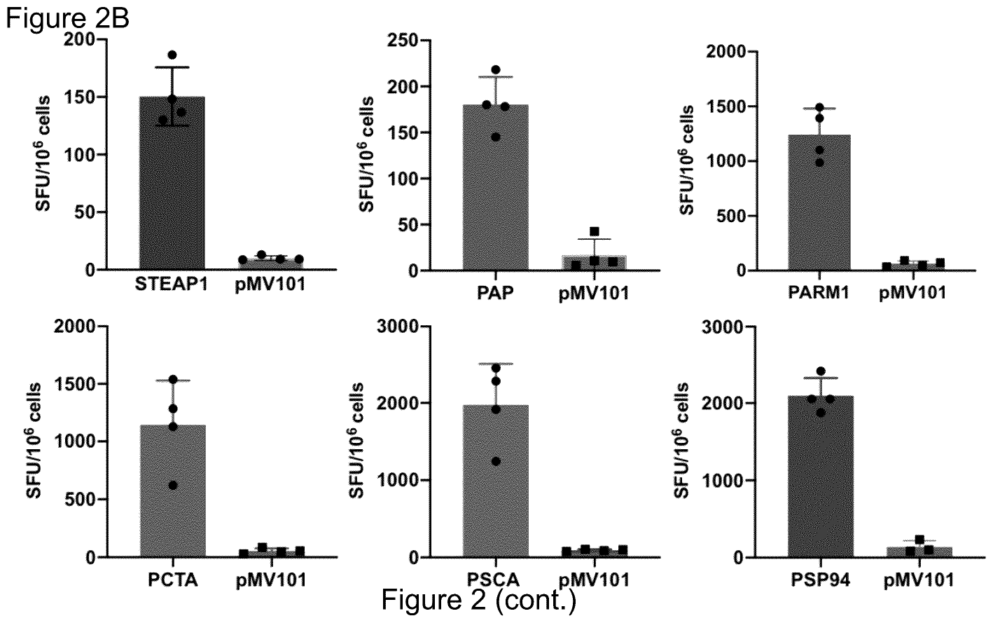

The immunization strategy adapted for vaccination dosage is presented in FIG. 2. Mice (C57BL/6, male) (n=4 per vaccine) were grouped and immunized with 50 μg of PCaA-SEV or pMV101 vector control followed by EP to enhance DNA delivery. In order to assess vaccination induced interferon gamma (IFN-γ)-producing T cells, ELISpot assays were performed using the spleen cells isolated from mice immunized with PCaA-SEV or pMV101 empty vector after stimulating with specific peptides (Bagarazzi M L et al., 2012, Transl Med., 4:155ra38). As shown in the schematic representation (FIG. 2A), one week after the third immunization (day 35), bulk splenocytes from mice immunized with the PCaA-SEV were obtained for ELISpot assay. Briefly, splenocytes from mice were ex vivo stimulated with PCaA peptides. IFN-γ produced by the cells specific to the antigens were reported as spot forming units (SFUs) per million cells (FIG. 2B). Notably, mice immunized with PSP94 DNA vaccine exhibited the most robust cellular responses. Similarly, PSCA, PCTA, and PARM1 vaccine groups also showed robust cellular responses to antigens. The splenocytes from mice immunized with STEAP1 and PAP-SEV registered low level of cellular immune responses compared to other vaccines. Collectively, these data demonstrated that PCaA-SEV induced antigen specific cellular immune responses effectively in mice.

PCaA-SEV Generated Significant Polyfunctionality in Both CD4⁺ and CD8⁺ T Cells

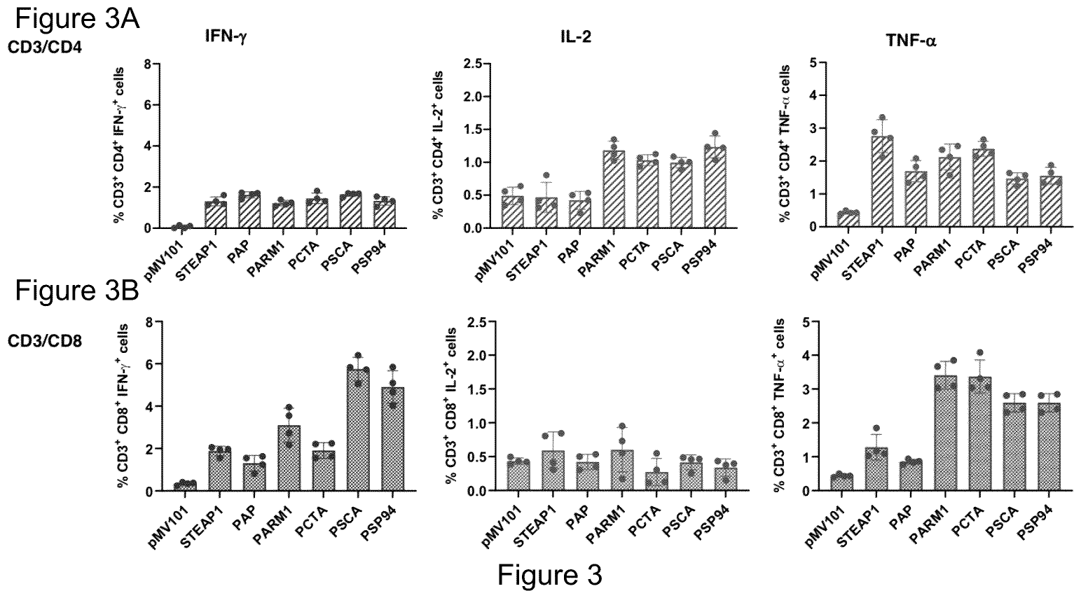

T cell polyfunctionality referred to the single-cell level co-expression of multiple functional molecules (Choi H et al., 2019, PLoS Negl Trop Dis., 13:e0007042). Upon understanding the potent PCaA-SEV induced immune responses through IFN-γ ELISpot assay, additional studies further determined the overall immunomodulatory effects of PCaA-SEV through staining of intracellular cytokines to evaluate the character of distinct functional CD8⁺/CD4⁺ T cell populations. For this purpose, splenocytes from C57BL/6 mice receiving three immunizations of PCaA vaccines or pMV101 were evaluated with the help of polychromatic flow cytometry. Specifically, bulk splenocytes were stimulated with vaccine specific PCaA peptides ex vivo. After permeabilization and fixation, cells were stained intracellularly with different fluorophore-tagged antibodies against IFN-γ, tumor necrosis factor-α (TNF-α), and interleukin 2 (IL-2). Stained cells were acquired using a FACS II flow cytometer and data were analyzed using FACS as described in the Materials and Methods, for determining CD4⁺ (FIG. 3A) as well as CD8⁺ T cells production (FIG. 3B) of the activated-state cytokines including IFN-γ, TNF-α, and IL-2. It was observed that mice immunized with different PCaA-SEV exhibited significantly higher frequency of CD4⁺ T cells secreting each intracellular cytokine upon stimulation with PCaA peptides. Similarly, CD8⁺ T cells isolated from the mice vaccinated with PCaA-SEV were also found to produce IFN-γ and TNF-α post PCaA peptides' stimulation. Thus, PCaA-SEV were noted to induce both cellular immunity to PCaA as well as polyfunctionality of antigen-specific T cells.

PCaA-SEV Vaccine Induced Humoral Immune Responses

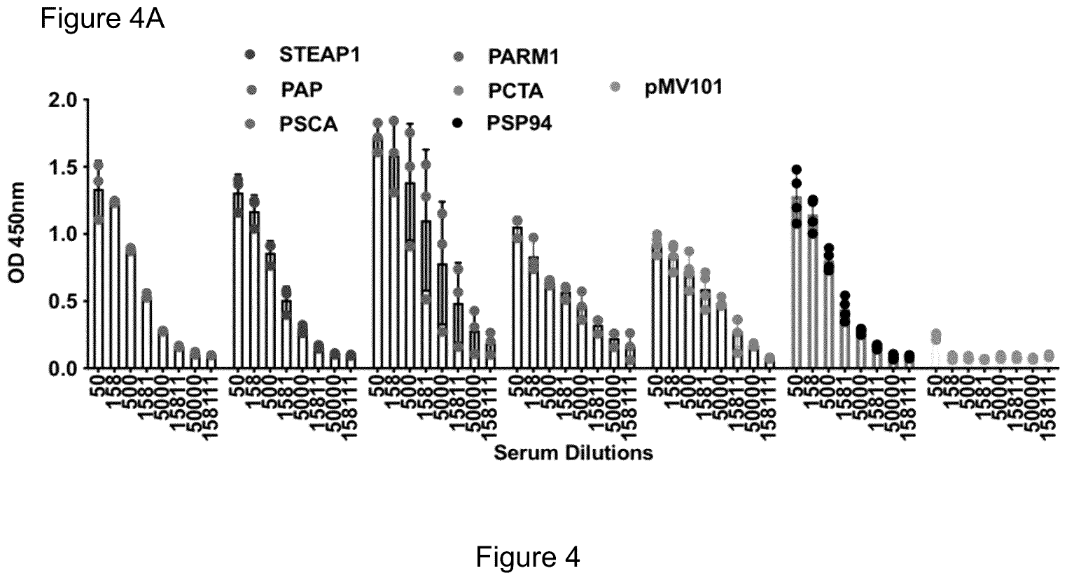

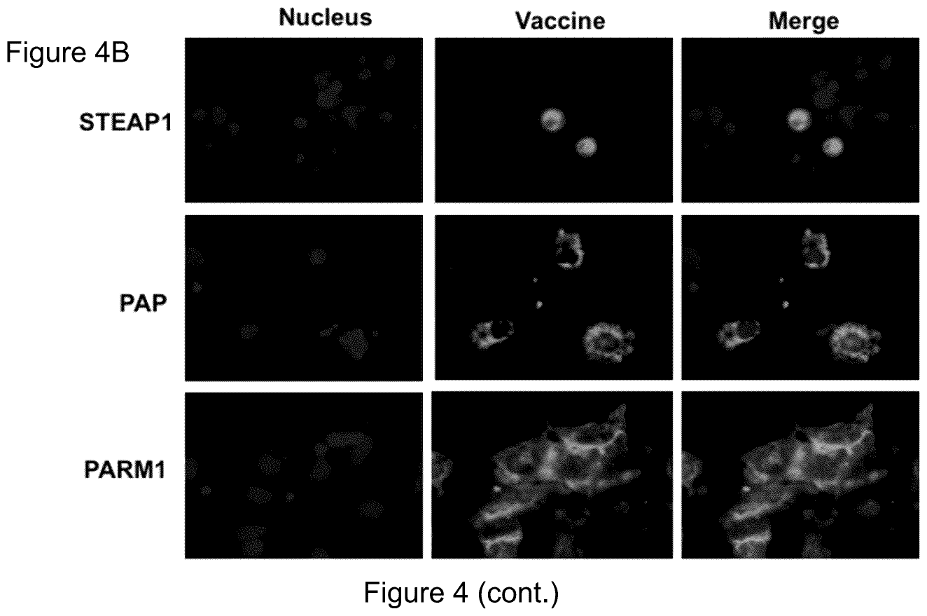

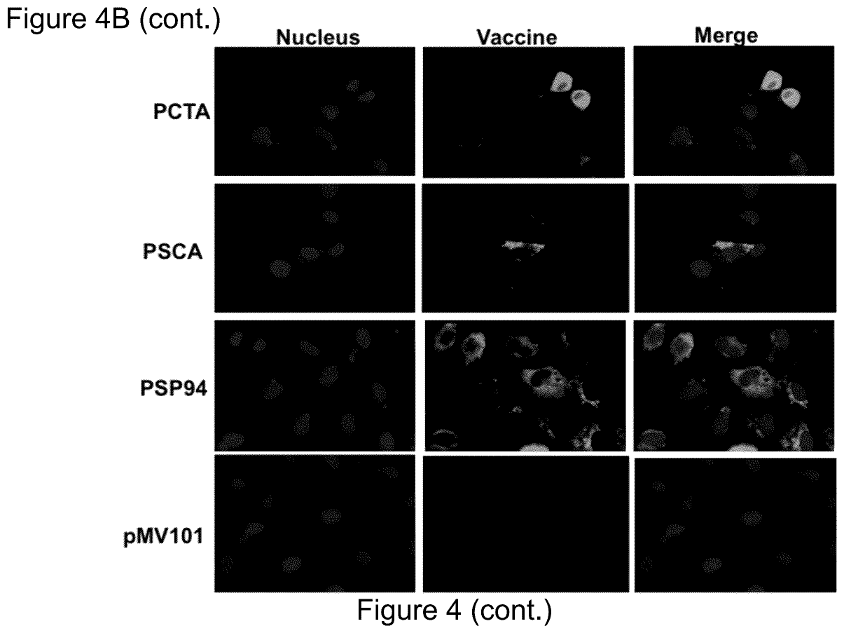

Next, the studies investigated the humoral immune responses of the PCaA-SEV. Firstly, the studies determined the antigen-specific antibody responses induced by each vaccine. Mice were immunized with the specific antigen and individual sera were collected for evaluating the reactivity of immunoglobulin G (IgG) antibodies in immune sera by ELISA (FIG. 4A). PCaA-SEV immune sera showed reactivity to the target antigen. Further, sera collected at day 35 were also tested by an immunofluorescence assay (IFA) to determine whether immune sera recognizes the production of specific antibodies against the target antigen (FIG. 4B). These data indicated that each of the individual components induced a humoral immune response, accompanied by strong binding. Importantly, these data also demonstrated that the synthetic DNA immunization along with electroporation induced a balanced antibody response similar to that induced by the cellular responses.

Intramuscular Administration of the PCaA-SEV Elicited Antitumor Immunity against Prostate Cancer

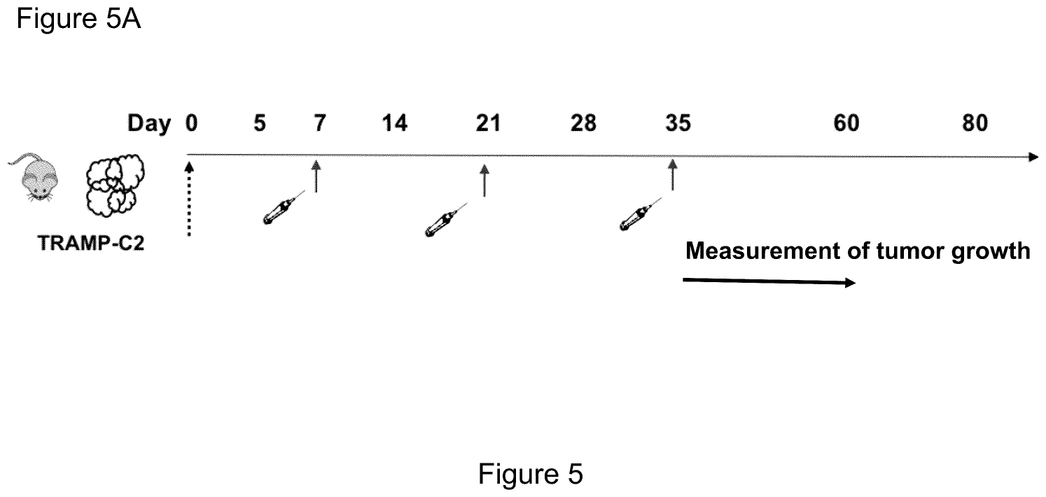

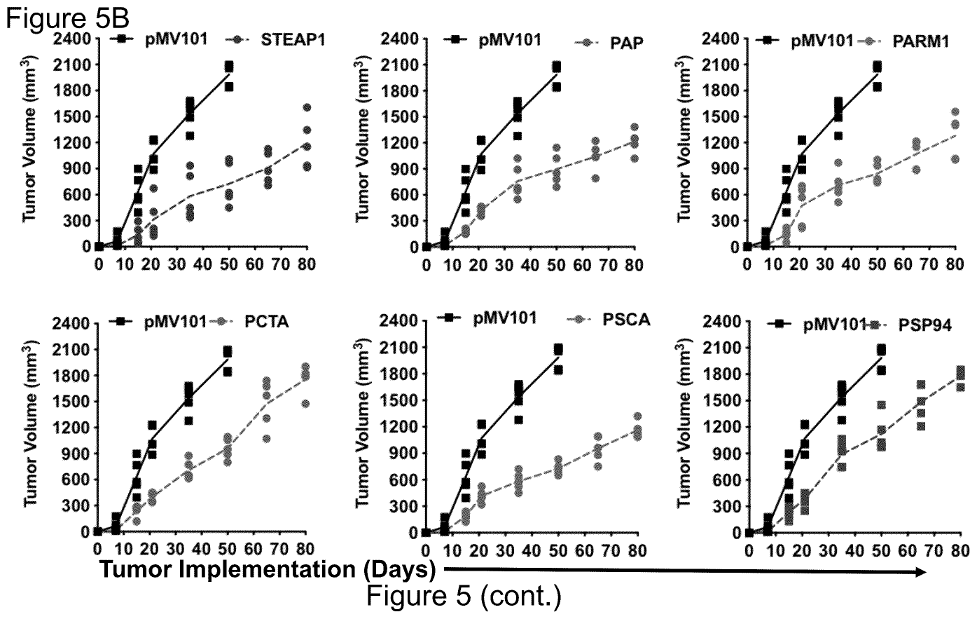

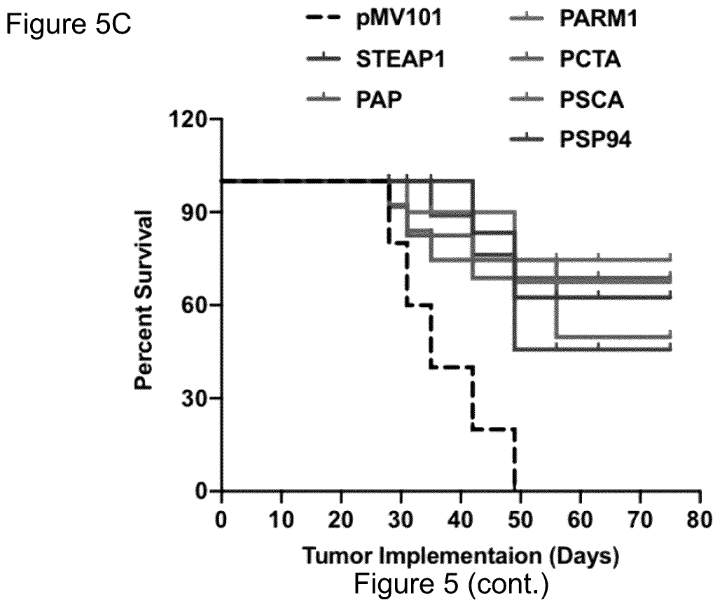

The assessment of the ability of vaccine-induced tumor-specific responses to provide protection against the disease in an animal challenge model was highly critical for establishing the therapeutic efficacy of any vaccine. For this reason, the present studies focused on evaluating the potential of PCaA-SEV by determining their ability to inhibit the growth of established prostate tumors. The schematic representation of the immunization as well as challenge strategies for the transgenic adenocarcinoma of the mouse prostate (TRAMP)-C2 mice model were shown (FIG. 5A). C57/BL6 mice were inoculated with TRAMP-C2 PCa cells as mentioned in the materials and methods section below. Seven days post inoculation with the PCa cells, the tumors were noted to be palpable. Then, the different groups of mice were immunized with 50 μg of PCaA-SEV or pMV101 vector, intramuscularly once weekly starting on day 7 for a total of three immunizations through EP-mediated delivery. Notably, vaccination with the different PCaA-SEV led to the delayed tumor progression in mice in comparison with the pMV101-vaccinated group (FIG. 5B). Thus, PCaA-SEV vaccination through EP enhanced delivery exerted potent effect against prostate tumor in TRAMP-C2 mice model, which was well evinced from the long-term survival of the PCaA vaccinated mice compared to the pMV101 vaccinated ones. (FIG. 5C).

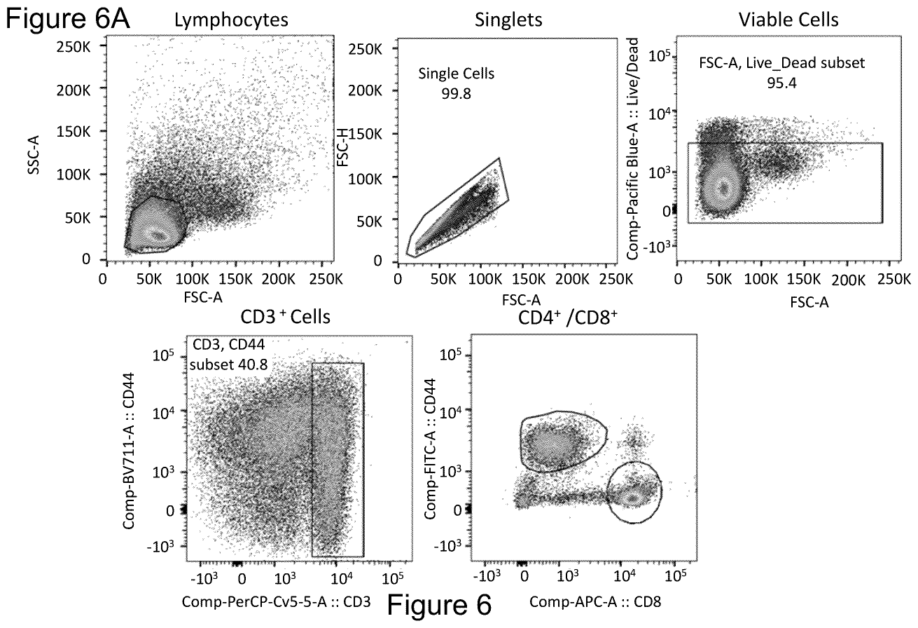

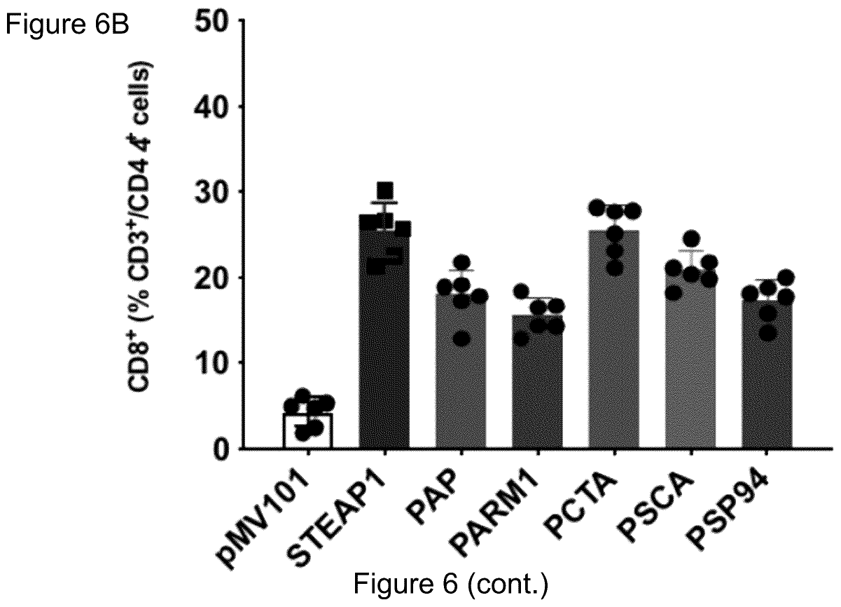

Further, the present studies investigated the percentage of CD8⁺ T cells of the total CD3⁺ and CD45⁺ cells in the tumor microenvironment in the PCaA-SEV vaccinated group of mice, 3 weeks after tumor inoculation (FIG. 6A). Upon analysis, an increase in T cell response against the prostate tumor was detected in the tumor microenvironment of vaccinated mice groups (FIG. 6B). Thus, PCaA-SEV vaccination led to enhanced infiltration of anti-tumor CD8⁺ T cells in the tumor microenvironment. Taken together, the present findings showed that EP mediated enhanced delivery of these DNA vaccines were able to generate PCaA specific CD8⁺ T cells and elevated their levels in the tumor microenvironment leading to improved survival of the mice bearing prostate tumor.

Cancer immunotherapy has emerged as a breakthrough treatment modality for diverse malignancies, through use of cancer vaccines, immune checkpoint inhibitors, adoptive cell therapy etc. Presently, different cancer vaccine platforms, such as peptide and recombinant virus vector-based vaccines, dendritic cell vaccines, engineered cellular vaccines, and idiotype vaccines have been established (Zhao Y et al., 2020, J Immunol., 204:518-530). In addition, recently emerged DNA vaccines represented another platform for treating different pathogens and evasive diseases, including cancer (Tebas P et al., 2017 N Engl J Med.; Trimble C L et al., 2015, Lancet., 386:2078-2088; Modjarrad K et al., 2019, Lancet Infect Dis., 19:1013-1022). DNA vaccines were highly flexible and versatile as they offered manipulation of vaccine targets through alteration of gene sequences of the delivered plasmid DNA (Lim M et al., 2020, Pharmaceutics., 12). Furthermore, these vaccines had the ability to induce potent antitumor cell-mediated immune responses against a diverse range of tumor antigens (Yan J et al., 2014, Cancer Gene Ther., 21:507-517). Although, there exist different tumor-specific antigens with unique expression on a lineage of distinct tumor cells, identifying the suitable tumor specific antigens to develop targeted therapy, causing minimal impairment to the normal cells, remained challenging (Yan J et al., 2014, Cancer Gene Ther., 21:507-517; Mahmoudian J et al., 2019, Cancer Immunol Immunother., 68:1039-1058).

STEAP1, PAP, PARM1, PCTA, PSCA, and PSP94 are different prostate specific proteins, which were found to be expressed in normal as well as malignant prostatic cancer tissues. STEAP1 is a cell surface protein, primarily located at cell-cell junctions, which was found to have limited expression in normal tissues, whereas high expression in primary PCa tissues (Yuan Y et al., 2019, J Ultrasound Med., 38:299-305). Increasing lines of evidence suggested STEAP1 as an effective biomarker and a potent target antigen for immunotherapy against prostatic malignancy (Challita-Eid P M et al., 2007, Cancer Res., 67:5798-5805). Cytotoxic T lymphocytes (CTLs) specific to STEAP1 led to the inhibition of transplantable prostate tumor cells' growth in vivo (Challita-Eid P M et al., 2007, Cancer Res., 67:5798-5805; Whiteland H et al., 2014, Clin Exp Metastasis., 31:909-920; Yamamoto T et al., 2013, Cell Res., 319: 2617-2626).

Another prostate tumor antigen, PAP is the target of Sipuleucel-T, the only FDA-approved anti-tumor vaccine (Wargowski E et al., 2018, J Immunother Cancer, 6:21). It is a secretory prostate-specific protein consisting of 354 amino acids. Over 95% of PCa tissues exerted elevated expression of PAP (Fujio K et al., 2015, Oncol Rep., 33:1585-1592). PARM1 codes for a 298-amino acid protein. Although low-level of PARM1 expression was detected in other organs besides prostate, its regulation by androgens seemed to be limited to this gland (Cornet A M et al., 2003, Prostate, 56:220-230). It was initially known as a highly induced gene in the prostate, post castration in rats. Elevated rat PARM1 expression was reported to cause enhanced telomerase function and the immortalization of prostate cancer cell lines, implying its role in the regulation of prostate cells' survival (Park J Y et al., 2013, Mol Endocrinol., 27:1871-1886).

Further, PCTA is another surface marker, found to be strongly linked with PCa (Gopalkrishnan R V et al., 2000, Oncogene, 19:4405-4416). It encodes a 35 kDa secreted protein having around 40% sequence homology with the N-amino terminal region of the S-type galactose-binding lectin (galectin) gene family members which are known to play role in tumorigenesis and metastasis (Su Z Z et al., 1996, Proc Natl Acad Sci USA, 93:7252-7257). PSCA, a glycosylphosphatidylinositol (GPI)-anchored cell surface protein, is a newly identified tumor associated antigen, which functions as an important marker for PCa (Wu D et al., 2020, Biomark Res., 8:3). This protein was reported to exhibit elevated expression level in above 80% local PCa cases and in all bone metastatic lesions (Kessler C et al., 2017, J Cancer Res Clin Oncol., 143:2025-2038). Notably, PSCA was considered as an effective marker for late stage PCa as its overexpression possess strong correlation with advancing tumor grade, stage, and progression to androgen independence (Kessler C et al., 2017, J Cancer Res Clin Oncol., 143:2025-2038). In addition, it anchored to cancer cell surface without exocytosis and therefore it was considered as a highly suitable target antigen for PCa immunotherapy (Mai T J et al., 2016, Braz J Med Biol Res., 49:e5620). PSP94, also known as prostatic inhibin or β-micro semino protein, was one of the most abundant proteins in semen along with PSA and PAP. As with other prostate-secreted proteins, PSP94 can leak into the blood upon benign or malignant prostate epithelial disruption and can be measured within serum. PSP94 was previously studied as a PCa blood biomarker (Reeves J R et al., 2006, Clin Cancer Res., 12:6018-6022; Shukeir N et al., 2005, Anticancer Drugs, 16:1045-1051). Consequently, targeting these proteins provided a new avenue for developing anti-tumor vaccines against PCa.

Notably, a number of pre-clinical and clinical studies have evaluated the role of these prostate specific antigens. For instance, previous studies reported that immunization with STEAP1 antigen through modified vaccinia virus Ankara vector in a murine subcutaneous tumor model led to the marked inhibition of PCa progression (Moreaux J et al., 2013, Br J Cancer, 109:676-685). Further, a renewed interest was generated in PAP due to its ability to predict intermediate to high-risk PCa cases and its success in PCa immunotherapy (Kong H Y et al., 2013, Biomol Ther (Seoul), 21:10-20). PAP-specific T-cell responses were reported to elicit and augment in human as well as animal models after antigen-specific immunization (Wagar L E et al., 2018, Genome Med., 10:73; Gerdts V et al., 2015, ILAR J., 56:53-62). Previous studies also showed a PAP encoded DNA vaccine to result in PAP-specific CD8⁺ T cell immune responses. Additionally, a phase I/II trial was conducted using a DNA vaccine encoding human PAP to treat 22 stage DO PCa patients. The findings revealed PSA values not to be dropped by more than 50% in the patients following treatment, however some patients were found to exhibit reduction in serum PSA rise rate (Yang B et al., 2014, Hum Vaccin Immunother., 10:3153-3164). Furthermore, PARM1 enabled the prostate cells to resist apoptosis via increased telomerase function (Cornet A M et al., 2003, Prostate, 56:220-230; Graham M K et al., 2017, Nat Rev Urol., 14:607-619).

In addition, anti-PSCA CAR-T cells have been considered to have potential to treat metastatic PCa (Priceman S J et al., 2018, Oncoimmunology, 7:e1380764). Furthermore, PCTA was also suggested to contribute as a low risk factor to the susceptibility of PCa in sporadic disease (Maier C et al., 2002, Eur Urol., 42:301-307). PSP94 was reported to play role in growth regulation and apoptosis induction in PCa cells. They were also known to regulate the levels of calcium during the hypercalcemic condition of malignancy (Reeves J R et al., 2006, Clin Cancer Res., 12:6018-6022). Further, its expression in radical prostatectomy tumor specimens was seemingly found to be linked with poor survival and thus signified its potent prognostic importance (Girvan A R et al., 2005, Urology, 65:719-723).

The present study evaluated the efficacy of these PCaA-SEV vaccines in the pre-clinical setting. Firstly, full-length gene sequences of different prostate specific antigens, namely STEAP1, PAP, PARM1, PSCA, PCTA, and PSP94, were synthetically designed. They were then successfully transfected, and their expressions were confirmed in HEK293T cells through Western blot analysis. The assessment of the PCaA vaccines demonstrated induction of cellular immunity as well as polyfunctionality of antigen-specific T cells. The highest cellular responses were observed with PSP94 DNA vaccine. In addition, PCTA and PSCA vaccine groups also exhibited markedly robust cellular immune responses. Both CD4⁺ and CD8⁺ induced elevated IFN-γ and TNF-α production, with more pronounced increase in case of CD8⁺ T cells. Further, the consensus sequences generated by these individual prostate antigens were capable of generating potent humoral immune responses to each antigen. Additionally, PCaA-SEV through EP mediated delivery was found to delay tumor progression and cause enhanced infiltration of anti-tumor CD8⁺ T cells in the tumor microenvironment, resulting in the long-term survival of the TRAMP-C2 mice and thus provided protection from prostate tumor.

The results presented here indicated that the selection of genes, based on multiple criteria, including expression pattern, as candidate vaccines combined with the advances in DNA delivery technologies provided a valuable immunotherapeutic approach to treat prostate cancer. Specifically, the present studies addressed the need in the art for novel treatment options for individuals who showed recurrence of prostate cancer after undergoing surgery and radiation treatments for early-stage cancer and also individuals with advanced stage of cancer. These groups greatly benefit from immunotherapeutic approaches (Zahm C D et al., 2017, Pharmacol Ther., 174:27-42; Liu M A et al., 2019, Vaccines (Basel), 7). Another advantage with the herein described strategy was the ability to combine vaccine candidates for simultaneous attack on multiple targets to suppress the tumor growth. Further, the immunotherapeutic approach can also be combined with other treatment modalities such as chemo and radiation therapies for the effective management of PCa. Therefore, additional studies are highly warranted to fully establish the clinical significance of these synthetic DNA vaccines targeting PCaA, which in turn result in the better clinical management of this neoplasm.

In summary, prostate cancer is a prevalent cancer in men and consists of both indolent and aggressive phenotypes. While active surveillance was recommended for the former, current treatments for the latter include surgery, radiation, chemo, and hormonal therapy. It has been observed that the recurrence in the treated patients was high and resulted in castration resistant prostate cancer for which treatment options are limited. This scenario has prompted the consideration of immunotherapy with synthetic DNA vaccines, as this approach generate antigen-specific tumor-killing immune cells. Utilizing a bioinformatics approach, synthetic enhanced DNA vaccine constructs were generated against STEAP1, PAP, PARM1, PSCA, PCTA, and PSP94. SEV for prostate cancer antigens were shown to elicit antigen-specific immune responses in mice and the anti-tumor activity was evident in a prostate tumor challenge mouse model. These data highlighted the advantages of using the synthetic DNA platform to target important tumor targets for immunotherapy of prostate cancer. Overall, the inclusion of multiple tumor antigens as vaccine candidates elicited an optimal response, and targeting multiple antigens conferred better protection in comparison to single antigen alone. Thus, the present studies demonstrated that synthetic DNA vaccines targeting different prostate specific antigens induce broader and improved immunity in subjects who are at high risk as well as advanced clinical stage of prostate cancer, compared to a single antigen approach.

The materials and methods employed in the present experimental examples are now described.

Cell Lines and Reagents

HEK293T, hepatocellular carcinoma cell line (HepG2), and TRAMP-C2 cells were procured from ATCC. These three cell types were maintained in D10 media: Dulbecco's Modified Eagle Medium (Invitrogen Life Science Technologies, San Diego, CA, USA) supplemented with 10% heat inactivated fetal calf serum (FCS), 3 mM glutamine, 100 U/mL penicillin, and 100 U/mL streptomycin. For mouse splenocyte cells, R10 media: (RPMI1640, Invitrogen Life Science Technologies, San Diego, CA, USA) supplemented with 10% heat-inactivated FCS, 3 mM glutamine, 100 U/mL penicillin, and 100 U/mL streptomycin was used. All the cells were maintained and grown in a 5% CO₂ regulated incubator set at 37° C. (Muthumani K et al., 2017, Cancer Immunol Immunother., 66:1577-1588).

Construction of Prostate Cancer Antigens-Synthetic Enhanced DNA Vaccine (PCaA-SEV)

Sequences of human PCaA, such as STEAP1, PAP, PARM1, PCTA, PSCA, and PSP94, were retrieved from NCBI database and immunogens were designed using codon- and RNA-optimized method as described before (Duperret E K et al., 2018, Cancer Res.; Bagarazzi M L et al., 2012, Transl Med., 4:155ra38; Choi H et al., 2019, PLoS Negl Trop Dis., 13: e0007042; Duperret E K et al., 2017, Clinical Cancer Research). Further, they were cloned individually into a pMV101 vector (GenScript, Piscataway, NJ, USA) under the control of the cytomegalovirus immediate-early promoter (Duperret E K et al., 2018, Cancer Res.; Perales-Puchalt A et al., 2019, JCI Insight., 4; Muthumani K et al., 2017, Cancer Immunol Immunother., 66:1577-1588). The SEV expressing STEAP1, PAP, PARM1, PCTA, PSCA, and PSP94 were designated as STEAP1 vaccine, PAP vaccine, PARM1 vaccine, PCTA vaccine, PSCA vaccine, and PSP94 vaccine, respectively, and together referred as PCaA-SEV henceforth.

Transfection and Expression of PCaA-SEV

HEK293T cells were seeded at a density of 6×10⁵ cells/well in six-well plates. After 24 hours, the cells were transfected with the above mentioned PCaA plasmids as well as pMV101 control plasmids using GeneJammer transfection reagent (Agilent Technologies, Santa Clara, CA, USA) as per the manufacturer's protocol. After 48 hours, the lysates of the transfected cells were collected, and Western blot analysis was performed for validating the antigen expression. Cell lysis was carried out using lysis buffer (50 mM HCl, 150 mM NaCl, 1% Nonidet P-40, 1% Triton X-100, 0.1% sodium dodecyl sulfate), and a cocktail of protease inhibitors (Roche, Basel, Switzerland). The supernatants obtained after cell lysis were then analyzed using sodium dodecyl sulfate-12% polyacrylamide gel electrophoresis. Subsequently, they were transferred to a polyvinylidene difluoride (PVDF) membrane and then incubated with primary antibodies against PCaA (i.e., anti-STEAP, anti-PCTA, anti-PSP94 (R&D Systems, Minneapolis, MN, USA); anti-PAP (Cell signaling Technology, Danvers, MA, USA); anti-PARM1, and anti-PSCA (ThermoFisher, Waltham, MA, USA)). Following this, the membrane was incubated with appropriate horseradish peroxidase (HRP) conjugated secondary antibodies (Li-Cor, Nebraska, USA). Then, the stripping of the membrane as done with the help of NewBlot Nitrocellulose 5× stripping buffer (Li-Cor, Nebraska, USA) followed by probing with 3-actin mouse monoclonal antibody (Li-Cor, Nebraska, USA). R-actin served as the loading control.

Enzyme Linked Immunosorbent Assay (ELISA)

For binding ELISA, firstly MaxiSorp high-binding 96-well ELISA plates (ThermoFisher, USA) were coated with different recombinant antigens at a concentration of 1 g/mL in PBS at 4° C. overnight. The plates were then washed 4 times with PBS containing 0.01% Tween-20 (PBST). Subsequently, blocking was done with 10% FBS in PBS for 1 hour at 37° C. Serum samples (collected from mice immunized with 50 μg of PCaA-SEV at day 35) were serially diluted (starting at 1:50, dilution factor 3.16) in PBS with 1% FBS. Then 100 μL of the diluted serum samples were added to the wells and incubated for 2 hours at 37° C. Following the incubation, the plates were washed 4 times with PBST and incubated with HRP-conjugated goat anti-mouse IgG (Sigma-Aldrich, St. Louis, MO, USA) for 1 hour at 37° C. Then 100 μL of 3,3′5,5′-tetramethylbenzidine (TMB) substrate (Sigma-Aldrich, St. Louis, MO, USA) was added to each well after the final wash and incubated for 10 minutes. The reaction was stopped by addition of 100 μL of 2N H₂SO₄ per well. Finally, the optical density of the plate was measured at 450 nm using an ELISA plate reader (Biotek, Winooski, VT, USA).

Immunofluorescence Analysis

In case of IFA, HepG2 liver cancer cells were seeded in 6-well cell culture plates on coverslips followed by transfection with PCaA-SEV as well as pMV101 empty vector as discussed (Choi H et al., 2019, PLoS Negl Trop Dis., 13). The cells were then incubated with sera collected from mice immunized with 50 μg of PCaA-SEV at day 35. Nuclear staining was done with 4′, 6-diamidino-2-phenylindole (DAPI) by incubating for 20 minutes at room temperature. Further, PCaA proteins were stained with the immunized sera (1:100) and then incubated with Alexa Fluor 488 dye. After each incubation step, washing with Phosphate-buffered saline (PBS) was carried out. Finally, the samples were mounted onto glass slides with the help of Fluoroshield mounting medium (Abcam, Cambridge, MA, USA) and then observed under a microscope (60× magnification) (Eclipse 80i, Nikon).

Animals, Study Approval, Plasmid Administration, and EP Delivery

Male C57BL/6 mice (five- to eight-week old) were procured from the Jackson Laboratory, ME, USA and vaccinated in a light-cycled, temperature- and humidity controlled animal facility. The mice were separated into different groups and immunized with 30 μL of 50 g pMV101 and 50 μg of different PCaA-SEV, intramuscularly, thrice at the intervals of 2-week followed by EP (CELLECTRA; Inovio Pharmaceuticals, Plymouth Meeting, PA, USA) (Choi H et al., 2019, PLoS Negl Trop Dis., 13). Specific pulsing parameters used for delivery were 2 pulses of 0.1 Amp constant current, 4 s apart and 52 ms in length (Broderick K E et al., 2017, Methods Mol Biol., 1499:193-200). The mice were housed in a barrier animal facility.

Isolation of Splenocyte and IFN-γ ELISpot Assay

The spleens of the mice were dissected and crushed using a Stomacher device (Seward, UK) and the splenocytes were filtered through a 40 m cell strainer (ThermoFisher, Waltham, MA, USA). For the lysis of red blood cells, the splenocytes were treated with Ammonium-Chloride-Potassium (ACK) lysis buffer (Quality Biologicals, MD, USA) for 5 minutes. Subsequently, Mouse IFN-γ ELISpot PLUS assay (Mabtech, Cincinnati, OH, USA) was carried out using the splenocytes resuspended in R10 as per the manufacturer's protocol. Precisely, splenocytes from PCaA-SEV or MV101 immunized mice were added at a density of 2×10⁵/well in plates and then incubated separately in the presence of only media (negative control), media along with cell activation cocktail (BioLegend, San Diego, CA, USA), pre-mixed phorbol 12-myristate-13-acetate (PMA) and ionomycin (positive control), and media with peptides with a final concentration of 1 μg/mL, for 18 hours at 37° C. in a 5% CO₂ regulated incubator. PCaA-SEV derived synthetic peptides were synthetized by Genscript, USA. The peptides were dissolved in DMSO and stored at −80° C. Bioinformatics approach using the SYFPEITHI website (syfpeithi.com) was utilized to define the dominant epitopes. Subsequently, upon addition of 5-bromo-4-chloro-3-indolyl-phosphate/nitro blue tetrazolium (BCIP/NBT) color development substrate (R&D Systems, Minneapolis, MN, USA), formation of spots were observed and the spot forming units (SFU) were then quantified with the help of automated ELISpot reader (CTL Limited, Ohio, USA).

Flow Cytometry and Intracellular Cytokine Staining Assay

Mouse splenocyte cells were seeded at a density of 2×10⁶ cells/well to a U-bottom 96-well plate (ThermoFisher, Waltham, MA, USA). The cells were then stimulated in the presence of media alone (negative control), or media with Cell Activation Cocktail (BioLegend, San Diego, CA, USA) containing pre-mixed PMA and ionomycin (positive control), or with media containing different PCaA peptides (1 μg/mL), where all the samples contained a protein transport inhibitor cocktail (eBioscience, San Diego, CA, USA) at 37° C. for 5 hours in a CO₂ regulated incubator. Following stimulation, the cells were washed with FACS buffer (PBS containing 0.1% sodium azide and 1% FBS) and then stained for the surface proteins using fluorochrome-conjugated antibodies. The cells were again washed with FACS buffer. Before staining with intracellular cytokines using fluorchrome-conjugated antibodies, cells were fixed and permeabilized with the help of BD Cytofix/Cytoperm (BD Biosciences, San Diego, CA, USA). Mouse antibodies used for staining in this assay were CD19 (V450; clone 1D3; BD Biosciences), CD3 (145-2C11; Biolegend), CD4 (RM4-5; eBioscience), CD8 (53-6.7; BD Biosciences), CD44 (IM7; BioLegend) IFN-γ (XMG1.2; Biolegend), TNF-α (MP6-XT22; eBioscience), IL-2 (JES6-SH4; eBioscience) and CD45 (30-F11, Biolegend). Live/dead exclusion was done with the Violet viability kit (Invitrogen Life Science Technologies, San Diego, CA, USA). All the data were acquired from an LSRII flow cytometer (BD Biosciences) and FlowJo software (Tree Star, Ashland, OR, USA) was used for analysis.

Tumor Challenge: Tumor Inoculation and Monitoring

For the tumor challenge study, C57BL/6 male mice were inoculated with 1.0×10⁶ TRAMP-C2 cells (in 200 μL PBS) subcutaneously in the right flank at day 0 followed by three vaccinations with PCaA-SEV or pMV101 at days 7, 21, and 35. Tumor masses were measured with a digital caliper, and tumor volumes were calculated approximating the tumor mass to a sphere, according to the following equation: {tumor volume=%/(length×width²)}. Moreover, the tumor-bearing mice were monitored daily for their survival. When the tumors obtained a size of 2 cm in diameter, they were humanely euthanized.

Statistics

All the statistical analyses were carried out with the help of GraphPad Prism software. Data are represented as the mean±Standard Error of the Mean (SEM). A two-tailed t test for studies with only 2 experimental groups and one-way ANOVA to test for experiments with more than 2 experimental groups were performed for determining the statistical significance. Statistical differences between vaccinated group compared to the pMV101 group were performed to levels of p<0.05.

### Example 2: Sequence Listings

TABLE 1
Sequence Listings.

| SEQ | | |
|---|---|---|
| ID | | |
| NO. | Name | Sequence |
| 1 | PAP Antigen | ATGCGGGCTGCTCCTCTGCTGCTGGCTCGGGCTGCTTCACTGTCCCT |
| | Nucleic Acid | GGGGTTCCTGTTTCTGCTGTTTTTCTGGCTGGATAGGTCTGTGCTGG |
| | Consensus | CCAAGGAGCTGAAGTTCGTGACCCTGGTGTTTCGCCACGGCGACAGG |
| | Sequence | TCCCCAATCGATACCTTCCCAACAGACCCCATCAAGGAGAGCTCCTG |
| | | GCCCCAGGGCTTTGGCCAGCTGACACAGCTGGGCATGGAGCAGCACT |
| | | ACGAGCTGGGCGAGTATATCAGGAAGAGATACCGGAAGTTCCTGAAC |
| | | GAGAGCTATAAGCACGAGCAGGTGTACATCCGCTCCACCGACGTGGA |
| | | TAGGACACTGATGTCTGCCATGACCAACCTGGCCGCCCTGTTTCCCC |
| | | CTGAGGGCGTGTCTATCTGGAATCCCATCCTGCTGTGGCAGCCTATC |
| | | CCAGTGCACACAGTGCCTCTGAGCGAGGATCAGCTGCTGTATCTGCC |
| | | ATTCAGAAACTGCCCCCGGTTTCAGGAGCTGGAGTCCGAGACACTGA |
| | | AGTCTGAGGAGTTCCAGAAGAGACTGCACCCCTACAAGGACTTTATC |
| | | GCCACACTGGGCAAGCTGAGCGGCCTGCACGGACAGGATCTGTTCGG |
| | | CATCTGGTCCAAGGTGTACGACCCTCTGTATTGTGAGTCTGTGCACA |
| | | ACTTCACCCTGCCAAGCTGGGCCACAGAGGATACCATGACAAAGCTG |
| | | AGGGAGCTGTCCGAGCTGTCTCTGCTGAGCCTGTATGGCATCCACAA |
| | | GCAGAAGGAGAAGTCCAGACTGCAGGGCGGCGTGCTGGTGAACGAGA |
| | | TCCTGAATCACATGAAGCGGGCCACCCAGATCCCTTCTTATAAGAAG |
| | | CTGATCATGTACAGCGCCCACGATACCACAGTGTCCGGCCTGCAGAT |
| | | GGCCCTGGACGTGTATAACGGCCTGCTGCCACCCTACGCATCCTGCC |
| | | ACCTGACCGAGCTGTATTTCGAGAAGGGCGAGTACTTTGTGGAGATG |
| | | TACTATCGCAATGAGACACAGCACGAGCCCTACCCTCTGATGCTGCC |
| | | AGGCTGCTCTCCTAGCTGTCCACTGGAGAGGTTCGCAGAGCTGGTGG |
| | | GACCCGTGATCCCTCAGGATTGGAGCACAGAATGTATGACCACTAAC |
| | | TCACACCAGGGGACAGAAGATAGCACTGATTGATAA |
| 2 | PAP Antigen | MRAAPLLLARAASLSLGFLFLLFFWLDRSVLAKELKFVTLVFRHGDR |
| | Amino Acid | SPIDTFPTDPIKESSWPQGFGQLTQLGMEQHYELGEYIRKRYRKFLN |
| | Consensus | ESYKHEQVYIRSTDVDRTLMSAMTNLAALFPPEGVSIWNPILLWQPI |
| | Sequence | PVHTVPLSEDQLLYLPFRNCPRFQELESETLKSEEFQKRLHPYKDFI |
| | | ATLGKLSGLHGQDLFGIWSKVYDPLYCESVHNFTLPSWATEDTMTKL |
| | | RELSELSLLSLYGIHKQKEKSRLQGGVLVNEILNHMKRATQIPSYKK |
| | | LIMYSAHDTTVSGLQMALDVYNGLLPPYASCHLTELYFEKGEYFVEM |
| | | YYRNETQHEPYPLMLPGCSPSCPLERFAELVGPVIPQDWSTECMTTN |
| | | SHQGTEDSTD |
| 3 | PAP Antigen | CGGGCTGCTCCTCTGCTGCTGGCTCGGGCTGCTTCACTGTCCCTGGG |
| | Nucleic Acid | GTTCCTGTTTCTGCTGTTTTTCTGGCTGGATAGGTCTGTGCTGGCCA |
| | Consensus | AGGAGCTGAAGTTCGTGACCCTGGTGTTTCGCCACGGCGACAGGTCC |
| | Sequence Lacking | CCAATCGATACCTTCCCAACAGACCCCATCAAGGAGAGCTCCTGGCC |
| | Start and Stop | CCAGGGCTTTGGCCAGCTGACACAGCTGGGCATGGAGCAGCACTACG |
| | Codons | AGCTGGGCGAGTATATCAGGAAGAGATACCGGAAGTTCCTGAACGAG |
| | | AGCTATAAGCACGAGCAGGTGTACATCCGCTCCACCGACGTGGATAG |
| | | GACACTGATGTCTGCCATGACCAACCTGGCCGCCCTGTTTCCCCCTG |
| | | AGGGCGTGTCTATCTGGAATCCCATCCTGCTGTGGCAGCCTATCCCA |
| | | GTGCACACAGTGCCTCTGAGCGAGGATCAGCTGCTGTATCTGCCATT |
| | | CAGAAACTGCCCCCGGTTTCAGGAGCTGGAGTCCGAGACACTGAAGT |
| | | CTGAGGAGTTCCAGAAGAGACTGCACCCCTACAAGGACTTTATCGCC |
| | | ACACTGGGCAAGCTGAGCGGCCTGCACGGACAGGATCTGTTCGGCAT |
| | | CTGGTCCAAGGTGTACGACCCTCTGTATTGTGAGTCTGTGCACAACT |
| | | TCACCCTGCCAAGCTGGGCCACAGAGGATACCATGACAAAGCTGAGG |
| | | GAGCTGTCCGAGCTGTCTCTGCTGAGCCTGTATGGCATCCACAAGCA |
| | | GAAGGAGAAGTCCAGACTGCAGGGCGGCGTGCTGGTGAACGAGATCC |
| | | TGAATCACATGAAGCGGGCCACCCAGATCCCTTCTTATAAGAAGCTG |
| | | ATCATGTACAGCGCCCACGATACCACAGTGTCCGGCCTGCAGATGGC |
| | | CCTGGACGTGTATAACGGCCTGCTGCCACCCTACGCATCCTGCCACC |
| | | TGACCGAGCTGTATTTCGAGAAGGGCGAGTACTTTGTGGAGATGTAC |
| | | TATCGCAATGAGACACAGCACGAGCCCTACCCTCTGATGCTGCCAGG |
| | | CTGCTCTCCTAGCTGTCCACTGGAGAGGTTCGCAGAGCTGGTGGGAC |
| | | CCGTGATCCCTCAGGATTGGAGCACAGAATGTATGACCACTAACTCA |
| | | CACCAGGGGACAGAAGATAGCACTGAT |
| 4 | PAP Antigen | RAAPLLLARAASLSLGFLFLLFFWLDRSVLAKELKFVTLVFRHGDRS |
| | Amino Acid | PIDTFPTDPIKESSWPQGFGQLTQLGMEQHYELGEYIRKRYRKFLNE |
| | Consensus | SYKHEQVYIRSTDVDRTLMSAMTNLAALFPPEGVSIWNPILLWQPIP |
| | Sequence Lacking | VHTVPLSEDQLLYLPFRNCPRFQELESETLKSEEFQKRLHPYKDFIA |
| | Start and Stop | TLGKLSGLHGQDLFGIWSKVYDPLYCESVHNFTLPSWATEDTMTKLR |
| | Codons | ELSELSLLSLYGIHKQKEKSRLQGGVLVNEILNHMKRATQIPSYKKL |
| | | IMYSAHDTTVSGLQMALDVYNGLLPPYASCHLTELYFEKGEYFVEMY |
| | | YRNETQHEPYPLMLPGCSPSCPLERFAELVGPVIPQDWSTECMTTNS |
| | | HQGTEDSTD |
| 5 | PARM1 Antigen | ATGGTGTATAAGACCCTGTTTGCCCTGTGCATTCTGACCGCCGGCTG |
| | Nucleic Acid | GAGAGTCCAGTCCCTGCCAACTTCCGCCCCTCTGAGCGTGTCCCTGC |
| | Consensus | CCACCAACATCGTGCCCCCTACCACAATCTGGACAAGCTCCCCTCAG |
| | Sequence | AACACCGACGCCGATACAGCCTCCCCATCTAATGGCACCCACAACAA |
| | | TAGCGTGCTGCCAGTGACCGCAAGCGCCCCAACATCCCTGCTGCCCA |
| | | AGAATATCTCTATCGAGAGCCGGGAGGAGGAGATCACCTCCCCAGGC |
| | | TCTAACTGGGAGGGCACAAATACCGACCCAAGCCCATCCGGCTTCTC |
| | | TAGCACCTCCGGCGGCGTGCACCTGACCACAACCCTGGAGGAGCACT |
| | | CCTCTGGAACCCCTGAGGCAGGAGTGGCAGCCACACTGTCTCAGAGC |
| | | GCCGCAGAGCCACCCACCCTGATCAGCCCCCAGGCCCCTGCCAGCTC |
| | | CCCATCTAGCCTGTCCACATCTCCTCCAGAGGTGTTTAGCGCCTCCG |
| | | TGACAACCAACCACTCCTCTACAGTGACCTCTACACAGCCAACCGGA |
| | | GCACCAACAGCACCAGAGAGCCCTACCGAGGAGAGCTCCTCTGACCA |
| | | CACCCCTACATCCCACGCAACCGCAGAGCCTGTGCCACAGGAGAAGA |
| | | CACCCCCTACAACCGTGTCTGGCAAAGTGATGTGCGAGCTGATCGAT |
| | | ATGGAGACAACCACAACCTTCCCTCGCGTGATCATGCAGGAGGTGGA |
| | | GCACGCACTGAGCTCCGGCAGCATCGCAGCCATCACCGTGACAGTGA |
| | | TCGCCGTGGTGCTGCTGGTGTTTGGCGTGGCCGCCTACCTGAAGATC |
| | | AGGCACTCTAGCTATGGCAGACTGCTGGACGACCACGACTACGGCTC |
| | | TTGGGGCAACTATAACAATCCTCTGTATGACGACTCATGATAA |
| 6 | PARM1 Antigen | MVYKTLFALCILTAGWRVQSLPTSAPLSVSLPTNIVPPTTIWTSSPQ |
| | Amino Acid | NTDADTASPSNGTHNNSVLPVTASAPTSLLPKNISIESREEEITSPG |
| | Consensus | SNWEGTNTDPSPSGFSSTSGGVHLTTTLEEHSSGTPEAGVAATLSQS |
| | Sequence | AAEPPTLISPQAPASSPSSLSTSPPEVFSASVTTNHSSTVTSTQPTG |
| | | APTAPESPTEESSSDHTPTSHATAEPVPQEKTPPTTVSGKVMCELID |
| | | METTTTFPRVIMQEVEHALSSGSIAAITVTVIAVVLLVFGVAAYLKI |
| | | RHSSYGRLLDDHDYGSWGNYNNPLYDDS |
| 7 | PARM1 Antigen | GTGTATAAGACCCTGTTTGCCCTGTGCATTCTGACCGCCGGCTGGAG |
| | Nucleic Acid | AGTCCAGTCCCTGCCAACTTCCGCCCCTCTGAGCGTGTCCCTGCCCA |
| | Consensus | CCAACATCGTGCCCCCTACCACAATCTGGACAAGCTCCCCTCAGAAC |
| | Sequence Lacking | ACCGACGCCGATACAGCCTCCCCATCTAATGGCACCCACAACAATAG |
| | Start and Stop | CGTGCTGCCAGTGACCGCAAGCGCCCCAACATCCCTGCTGCCCAAGA |
| | Codon | ATATCTCTATCGAGAGCCGGGAGGAGGAGATCACCTCCCCAGGCTCT |
| | | AACTGGGAGGGCACAAATACCGACCCAAGCCCATCCGGCTTCTCTAG |
| | | CACCTCCGGCGGCGTGCACCTGACCACAACCCTGGAGGAGCACTCCT |
| | | CTGGAACCCCTGAGGCAGGAGTGGCAGCCACACTGTCTCAGAGCGCC |
| | | GCAGAGCCACCCACCCTGATCAGCCCCCAGGCCCCTGCCAGCTCCCC |
| | | ATCTAGCCTGTCCACATCTCCTCCAGAGGTGTTTAGCGCCTCCGTGA |
| | | CAACCAACCACTCCTCTACAGTGACCTCTACACAGCCAACCGGAGCA |
| | | CCAACAGCACCAGAGAGCCCTACCGAGGAGAGCTCCTCTGACCACAC |
| | | CCCTACATCCCACGCAACCGCAGAGCCTGTGCCACAGGAGAAGACAC |
| | | CCCCTACAACCGTGTCTGGCAAAGTGATGTGCGAGCTGATCGATATG |
| | | GAGACAACCACAACCTTCCCTCGCGTGATCATGCAGGAGGTGGAGCA |
| | | CGCACTGAGCTCCGGCAGCATCGCAGCCATCACCGTGACAGTGATCG |
| | | CCGTGGTGCTGCTGGTGTTTGGCGTGGCCGCCTACCTGAAGATCAGG |
| | | CACTCTAGCTATGGCAGACTGCTGGACGACCACGACTACGGCTCTTG |
| | | GGGCAACTATAACAATCCTCTGTATGACGACTCA |
| 8 | PARM1 Antigen | VYKTLFALCILTAGWRVQSLPTSAPLSVSLPTNIVPPTTIWTSSPQN |
| | Amino Acid | TDADTASPSNGTHNNSVLPVTASAPTSLLPKNISIESREEEITSPGS |
| | Consensus | NWEGTNTDPSPSGFSSTSGGVHLTTTLEEHSSGTPEAGVAATLSQSA |
| | Sequence Lacking | AEPPTLISPQAPASSPSSLSTSPPEVFSASVTTNHSSTVTSTQPTGA |
| | Start and Stop | PTAPESPTEESSSDHTPTSHATAEPVPQEKTPPTTVSGKVMCELIDM |
| | Codon | ETTTTFPRVIMQEVEHALSSGSIAAITVTVIAVVLLVFGVAAYLKIR |
| | | HSSYGRLLDDHDYGSWGNYNNPLYDDS |
| 9 | PCTA Antigen | ATGCTGTCACTGAACAACCTGCAGAACATCATCTATAACCCTGTCAT |
| | Nucleic Acid | CCCCTTCGTCGGAACCATCCCAGACCAGCTGGACCCCGGCACCCTGA |
| | Consensus | TCGTGATCAGGGGCCACGTGCCAAGCGACGCCGATAGATTCCAGGTG |
| | Sequence | GACCTGCAGAACGGCAGCTCCGTGAAGCCTCGGGCCGATGTGGCCTT |
| | | CCACTTTAATCCAAGGTTTAAGAGAGCCGGCTGCATCGTGTGCAACA |
| | | CACTGATCAATGAGAAGTGGGGCAGGGAGGAGATCACCTACGACACA |
| | | CCCTTCAAGAGAGAGAAGTCCTTTGAGATCGTGATCATGGTGCTGAA |
| | | GGATAAGTTCCAGGTGGCCGTGAACGGCAAGCACACCCTGCTGTACG |
| | | GCCACAGGATCGGCCCTGAGAAGATCGACACACTGGGCATCTATGGC |
| | | AAGGTCAATATCCACAGCATCGGCTTCTCCTTTTCTAGCGATCTGCA |
| | | GTCTACCCAGGCCTCCTCTCTGGAGCTGACAGAGATCGTGAGAGAGA |
| | | ACGTGCCTAAGAGCGGCACCCCACAGCTGTCCCTGCCCTTCGCCGCA |
| | | CGGCTGAATACCCCAATGGGACCTGGAAGGACAGTGGTGGTGCAGGG |
| | | AGAGGTGAACGCCAATGCCAAGTCTTTTAACGTGGACCTGCTGGCCG |
| | | GCAAGAGCAAGGATATCGCCCTGCACCTGAACCCCCGGCTGAATATC |
| | | AAGGCCTTCGTGCGCAACTCCTTTCTGCAGGAGTCTTGGGGCGAGGA |
| | | GGAGCGCAATATCACATCTTTCCCCTTCAGCCCCGGCATGTACTTCG |
| | | AGATGATCATCTATTGCGACGTGCGGGAGTTTAAGGTGGCCGTGAAT |
| | | GGCGTGCACTCCCTGGAGTATAAGCACCGCTTCAAGGAGCTGAGTAG |
| | | CATTGACACCCTGGAGATTAACGGAGACATTCACCTGCTGGAAGTGC |
| | | GGTCCTGGTGATAA |
| 10 | PCTA Antigen | MLSLNNLQNIIYNPVIPFVGTIPDQLDPGTLIVIRGHVPSDADRFQV |
| | Amino Acid | DLQNGSSVKPRADVAFHFNPRFKRAGCIVCNTLINEKWGREEITYDT |
| | Consensus | PFKREKSFEIVIMVLKDKFQVAVNGKHTLLYGHRIGPEKIDTLGIYG |
| | Sequence | KVNIHSIGFSFSSDLQSTQASSLELTEIVRENVPKSGTPQLSLPFAA |
| | | RLNTPMGPGRTVVVQGEVNANAKSFNVDLLAGKSKDIALHLNPRLNI |
| | | KAFVRNSFLQESWGEEERNITSFPFSPGMYFEMIIYCDVREFKVAVN |
| | | GVHSLEYKHRFKELSSIDTLEINGDIHLLEVRSW |
| 11 | PCTA Antigen | CTGTCACTGAACAACCTGCAGAACATCATCTATAACCCTGTCATCCC |
| | Nucleic Acid | CTTCGTCGGAACCATCCCAGACCAGCTGGACCCCGGCACCCTGATCG |
| | Consensus | TGATCAGGGGCCACGTGCCAAGCGACGCCGATAGATTCCAGGTGGAC |
| | Sequence Lacking | CTGCAGAACGGCAGCTCCGTGAAGCCTCGGGCCGATGTGGCCTTCCA |
| | Start and Stop | CTTTAATCCAAGGTTTAAGAGAGCCGGCTGCATCGTGTGCAACACAC |
| | Codon | TGATCAATGAGAAGTGGGGCAGGGAGGAGATCACCTACGACACACCC |
| | | TTCAAGAGAGAGAAGTCCTTTGAGATCGTGATCATGGTGCTGAAGGA |
| | | TAAGTTCCAGGTGGCCGTGAACGGCAAGCACACCCTGCTGTACGGCC |
| | | ACAGGATCGGCCCTGAGAAGATCGACACACTGGGCATCTATGGCAAG |
| | | GTCAATATCCACAGCATCGGCTTCTCCTTTTCTAGCGATCTGCAGTC |
| | | TACCCAGGCCTCCTCTCTGGAGCTGACAGAGATCGTGAGAGAGAACG |
| | | TGCCTAAGAGCGGCACCCCACAGCTGTCCCTGCCCTTCGCCGCACGG |
| | | CTGAATACCCCAATGGGACCTGGAAGGACAGTGGTGGTGCAGGGAGA |
| | | GGTGAACGCCAATGCCAAGTCTTTTAACGTGGACCTGCTGGCCGGCA |
| | | AGAGCAAGGATATCGCCCTGCACCTGAACCCCCGGCTGAATATCAAG |
| | | GCCTTCGTGCGCAACTCCTTTCTGCAGGAGTCTTGGGGCGAGGAGGA |
| | | GCGCAATATCACATCTTTCCCCTTCAGCCCCGGCATGTACTTCGAGA |
| | | TGATCATCTATTGCGACGTGCGGGAGTTTAAGGTGGCCGTGAATGGC |
| | | GTGCACTCCCTGGAGTATAAGCACCGCTTCAAGGAGCTGAGTAGCAT |
| | | TGACACCCTGGAGATTAACGGAGACATTCACCTGCTGGAAGTGCGGT |
| | | CCTGG |
| 12 | PCTA Antigen | LSLNNLQNIIYNPVIPFVGTIPDQLDPGTLIVIRGHVPSDADRFQVD |
| | Amino Acid | LQNGSSVKPRADVAFHFNPRFKRAGCIVCNTLINEKWGREEITYDTP |
| | Consensus | FKREKSFEIVIMVLKDKFQVAVNGKHTLLYGHRIGPEKIDTLGIYGK |
| | Sequence Lacking | VNIHSIGFSFSSDLQSTQASSLELTEIVRENVPKSGTPQLSLPFAAR |
| | Start and Stop | LNTPMGPGRTVVVQGEVNANAKSENVDLLAGKSKDIALHLNPRLNIK |
| | Codon | AFVRNSFLQESWGEEERNITSFPFSPGMYFEMIIYCDVREFKVAVNG |
| | | VHSLEYKHRFKELSSIDTLEINGDIHLLEVRSW |
| 13 | PSCA Antigen | ATGAAAGCCGTCCTGCTGGCACTGCTGATGGCCGGCCTGGCACTGCA |
| | Nucleic Acid | GCCCGGAACCGCTCTGCTGTGCTACTCCTGTAAAGCCCAGGTGAGCA |
| | Consensus | ACGAGGACTGCCTGCAGGTGGAGAATTGTACCCAGCTGGGCGAGCAG |
| | Sequence | TGCTGGACAGCCAGGATCAGAGCCGTGGGCCTGCTGACCGTGATCAG |
| | | CAAGGGCTGCTCCCTGAACTGCGTGGACGATTCTCAGGATTACTATG |
| | | TGGGCAAGAAGAACATCACCTGCTGTGACACAGACCTGTGCAATGCC |
| | | AGCGGCGCCCACGCCCTGCAGCCCGCCGCCGCAATCCTGGCTCTGCT |
| | | GCCCGCCCTGGGACTGCTGCTGTGGGGACCTGGACAGCTGTGATAA |
| 14 | PSCA Antigen | MKAVLLALLMAGLALQPGTALLCYSCKAQVSNEDCLQVENCTQLGEQ |
| | Amino Acid | CWTARIRAVGLLTVISKGCSLNCVDDSQDYYVGKKNITCCDTDLCNA |
| | Consensus | SGAHALQPAAAILALLPALGLLLWGPGQL |
| | Sequence | |
| 15 | PSCA Antigen | AAAGCCGTCCTGCTGGCACTGCTGATGGCCGGCCTGGCACTGCAGCC |
| | Nucleic Acid | CGGAACCGCTCTGCTGTGCTACTCCTGTAAAGCCCAGGTGAGCAACG |
| | Consensus | AGGACTGCCTGCAGGTGGAGAATTGTACCCAGCTGGGCGAGCAGTGC |
| | Sequence Lacking | TGGACAGCCAGGATCAGAGCCGTGGGCCTGCTGACCGTGATCAGCAA |
| | Start and Stop | GGGCTGCTCCCTGAACTGCGTGGACGATTCTCAGGATTACTATGTGG |
| | Codon | GCAAGAAGAACATCACCTGCTGTGACACAGACCTGTGCAATGCCAGC |
| | | GGCGCCCACGCCCTGCAGCCCGCCGCCGCAATCCTGGCTCTGCTGCC |
| | | CGCCCTGGGACTGCTGCTGTGGGGACCTGGACAGCTG |
| 16 | PSCA Antigen | KAVLLALLMAGLALQPGTALLCYSCKAQVSNEDCLQVENCTQLGEQC |
| | Amino Acid | WTARIRAVGLLTVISKGCSLNCVDDSQDYYVGKKNITCCDTDLCNAS |
| | Consensus | GAHALQPAAAILALLPALGLLLWGPGQL |
| | Sequence Lacking | |
| | Start and Stop | |
| | Codon | |
| 17 | PSP94 Antigen | ATGAACGTGCTGCTGGGCTCCGTGGTCATTTTCGCTACATTCGTCAC |
| | Nucleic Acid | TCTGTGCAACGCTTCCTGTTACTTTATTCCAAACGAGGGGGTGCCCG |
| | Consensus | GCGACTCTACCAGGAAGTGCATGGATCTGAAGGGCAACAAGCACCCT |
| | Sequence | ATCAATAGCGAGTGGCAGACCGACAACTGTGAGACATGCACATGTTA |
| | | CGAGACAGAGATCAGCTGCTGTACCCTGGTGTCCACACCCGTGGGCT |
| | | ATGACAAGGATAATTGCCAGAGAATCTTCAAGAAGGAGGATTGTAAG |
| | | TACATCGTGGTGGAGAAAAAGGACCCCAAAAAGACCTGTTCAGTCAG |
| | | CGAATGGATTATTTGATAA |
| 18 | PSP94 Antigen | MNVLLGSVVIFATFVTLCNASCYFIPNEGVPGDSTRKCMDLKGNKHP |
| | Amino Acid | INSEWQTDNCETCTCYETEISCCTLVSTPVGYDKDNCQRIFKKEDCK |
| | Consensus | YIVVEKKDPKKTCSVSEWII |
| | Sequence | |
| 19 | PSP94 Antigen | AACGTGCTGCTGGGCTCCGTGGTCATTTTCGCTACATTCGTCACTCT |
| | Nucleic Acid | GTGCAACGCTTCCTGTTACTTTATTCCAAACGAGGGGGTGCCCGGCG |
| | Consensus | ACTCTACCAGGAAGTGCATGGATCTGAAGGGCAACAAGCACCCTATC |
| | Sequence Lacking | AATAGCGAGTGGCAGACCGACAACTGTGAGACATGCACATGTTACGA |
| | Start and Stop | GACAGAGATCAGCTGCTGTACCCTGGTGTCCACACCCGTGGGCTATG |
| | Codon | ACAAGGATAATTGCCAGAGAATCTTCAAGAAGGAGGATTGTAAGTAC |
| | | ATCGTGGTGGAGAAAAAGGACCCCAAAAAGACCTGTTCAGTCAGCGA |
| | | ATGGATTATT |
| 20 | PSP94 Antigen | NVLLGSVVIFATFVTLCNASCYFIPNEGVPGDSTRKCMDLKGNKHPI |
| | Amino Acid | NSEWQTDNCETCTCYETEISCCTLVSTPVGYDKDNCQRIFKKEDCKY |
| | Consensus | IVVEKKDPKKTCSVSEWII |
| | Sequence Lacking | |
| | Start and Stop | |
| | Codon | |
| 21 | STEAP1 Antigen | ATGGAATCAAGGAAGGACATCACCAATCAGGAGGAACTGTGGAAAAT |
| | Nucleic Acid | GAAGCCCCGAAGAAATCTGGAAGAGGACGACTACCTGCATAAGGACA |
| | Consensus | CCGGCGAGACATCCATGCTGAAGAGGCCAGTGCTGCTGCACCTGCAC |
| | Sequence | CAGACCGCACACGCCGACGAGTTTGATTGCCCCTCTGAGCTGCAGCA |
| | | CACACAGGAGCTGTTCCCACAGTGGCACCTGCCCATCAAGATCGCCG |
| | | CCATCATCGCCAGCCTGACCTTTCTGTACACACTGCTGAGAGAAGTG |
| | | ATCCACCCCCTGGCCACCTCCCACCAGCAGTACTTCTATAAGATCCC |
| | | TATCCTGGTCATCAACAAGGTGCTGCCAATGGTGTCCATCACACTGC |
| | | TGGCCCTGGTGTACCTGCCTGGCGTGATCGCCGCCATCGTGCAGCTG |
| | | CACAATGGCACCAAGTATAAGAAGTTTCCACACTGGCTGGATAAGTG |
| | | GATGCTGACAAGGAAGCAGTTCGGCCTGCTGTCTTTCTTTTTCGCCG |
| | | TGCTGCACGCCATCTACAGCCTGTCCTATCCCATGAGGAGAAGCTAC |
| | | CGGTATAAGCTGCTGAACTGGGCCTACCAGCAGGTGCAGCAGAATAA |
| | | GGAGGACGCCTGGATCGAGCACGACGTGTGGCGCATGGAGATCTACG |
| | | TGAGCCTGGGAATCGTGGGCCTGGCAATCCTGGCCCTGCTGGCAGTG |
| | | ACCTCTATCCCTTCTGTGAGCGACTCCCTGACATGGCGGGAGTTTCA |
| | | CTACATCCAGTCTAAGCTGGGCATCGTGAGCCTGCTGCTGGGCACCA |
| | | TCCACGCCCTGATCTTTGCCTGGAACAAGTGGATCGATATCAAGCAG |
| | | TTCGTGTGGTATACCCCTCCCACCTTCATGATCGCCGTGTTCCTGCC |
| | | CATCGTGGTGCTGATCTTTAAGAGCATCCTGTTCCTGCCTTGTCTGC |
| | | GCAAGAAGATCCTGAAGATTCGGCACGGGTGGGAAGATGTGACTAAG |
| | | ATCAATAAGACAGAGATTTGTAGCCAGCTGTGATAA |
| 22 | STEAP1 Antigen | MESRKDITNQEELWKMKPRRNLEEDDYLHKDTGETSMLKRPVLLHLH |
| | Amino Acid | QTAHADEFDCPSELQHTQELFPQWHLPIKIAAIIASLTFLYTLLREV |
| | Consensus | IHPLATSHQQYFYKIPILVINKVLPMVSITLLALVYLPGVIAAIVQL |
| | Sequence | HNGTKYKKFPHWLDKWMLTRKQFGLLSFFFAVLHAIYSLSYPMRRSY |
| | | RYKLLNWAYQQVQQNKEDAWIEHDVWRMEIYVSLGIVGLAILALLAV |
| | | TSIPSVSDSLTWREFHYIQSKLGIVSLLLGTIHALIFAWNKWIDIKQ |
| | | FVWYTPPTFMIAVFLPIVVLIFKSILFLPCLRKKILKIRHGWEDVTK |
| | | INKTEICSQL |
| 23 | STEAP1 Antigen | GAATCAAGGAAGGACATCACCAATCAGGAGGAACTGTGGAAAATGAA |
| | Nucleic Acid | GCCCCGAAGAAATCTGGAAGAGGACGACTACCTGCATAAGGACACCG |
| | Consensus | GCGAGACATCCATGCTGAAGAGGCCAGTGCTGCTGCACCTGCACCAG |
| | Sequence | ACCGCACACGCCGACGAGTTTGATTGCCCCTCTGAGCTGCAGCACAC |
| | Lacking | ACAGGAGCTGTTCCCACAGTGGCACCTGCCCATCAAGATCGCCGCCA |
| | Start and | TCATCGCCAGCCTGACCTTTCTGTACACACTGCTGAGAGAAGTGATC |
| | Stop | CACCCCCTGGCCACCTCCCACCAGCAGTACTTCTATAAGATCCCTAT |
| | Codon | CCTGGTCATCAACAAGGTGCTGCCAATGGTGTCCATCACACTGCTGG |
| | | CCCTGGTGTACCTGCCTGGCGTGATCGCCGCCATCGTGCAGCTGCAC |
| | | AATGGCACCAAGTATAAGAAGTTTCCACACTGGCTGGATAAGTGGAT |
| | | GCTGACAAGGAAGCAGTTCGGCCTGCTGTCTTTCTTTTTCGCCGTGC |
| | | TGCACGCCATCTACAGCCTGTCCTATCCCATGAGGAGAAGCTACCGG |
| | | TATAAGCTGCTGAACTGGGCCTACCAGCAGGTGCAGCAGAATAAGGA |
| | | GGACGCCTGGATCGAGCACGACGTGTGGCGCATGGAGATCTACGTGA |
| | | GCCTGGGAATCGTGGGCCTGGCAATCCTGGCCCTGCTGGCAGTGACC |
| | | TCTATCCCTTCTGTGAGCGACTCCCTGACATGGCGGGAGTTTCACTA |
| | | CATCCAGTCTAAGCTGGGCATCGTGAGCCTGCTGCTGGGCACCATCC |
| | | ACGCCCTGATCTTTGCCTGGAACAAGTGGATCGATATCAAGCAGTTC |
| | | GTGTGGTATACCCCTCCCACCTTCATGATCGCCGTGTTCCTGCCCAT |
| | | CGTGGTGCTGATCTTTAAGAGCATCCTGTTCCTGCCTTGTCTGCGCA |
| | | AGAAGATCCTGAAGATTCGGCACGGGTGGGAAGATGTGACTAAGATC |
| | | AATAAGACAGAGATTTGTAGCCAGCTG |
| 24 | STEAP1 Antigen | ESRKDITNQEELWKMKPRRNLEEDDYLHKDTGETSMLKRPVLLHLHQ |
| | Amino Acid | TAHADEFDCPSELQHTQELFPQWHLPIKIAAIIASLTFLYTLLREVI |
| | Consensus | HPLATSHQQYFYKIPILVINKVLPMVSITLLALVYLPGVIAAIVQLH |
| | Sequence | NGTKYKKFPHWLDKWMLTRKQFGLLSFFFAVLHAIYSLSYPMRRSYR |
| | Lacking | YKLLNWAYQQVQQNKEDAWIEHDVWRMEIYVSLGIVGLAILALLAVT |
| | Start and | SIPSVSDSLTWREFHYIQSKLGIVSLLLGTIHALIFAWNKWIDIKQF |
| | Stop | VWYTPPTFMIAVFLPIVVLIFKSILFLPCLRKKILKIRHGWEDVTKI |
| | Codon | NKTEICSQL |
| 25 | IgE Leader | ATGGACTGGACATGGATTCTGTTTCTGGTCGCTGCCGCAACCCGCGT |
| | Sequence- | GCATTCA |
| | Nucleic Acid | |
| 26 | IgE Leader | MDWTWILFLVAAATRVHS |
| | Sequence- | |
| | Amino Acid | |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A nucleic acid molecule comprising a coding sequence encoding one or more proteins selected from the group consisting of:
a) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof,
b) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof,
c) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof,
d) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof,
e) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof,
f) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof,
g) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof,
h) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof,
i) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof,
j) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof,
k) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and
l) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

2. The nucleic acid molecule of claim 1, wherein the nucleic acid molecule encodes one or more proteins selected from the group consisting of: a), b), c), d), e), f), g), h), i), j), k), and l).

3. The nucleic acid molecule of claim 1, wherein the nucleic acid molecule encodes one or more proteins selected from the group consisting of:
i) at least one selected from either elements a) or b);
ii) at least one selected from either elements c) or d);
iii) at least one selected from either elements e) or f);
iv) at least one selected from either elements g) or h);
v) at least one selected from either elements i) or j); and
vi) at least one selected from either elements k) or l).

4. The nucleic acid molecule of any one of claims 1-3, wherein the nucleic acid molecule encodes one or more proteins selected from the group consisting of: SEQ ID NO:2; SEQ ID NO:4; SEQ ID NO:6; SEQ ID NO:8; SEQ ID NO:10; SEQ ID NO: 12; SEQ ID NO: 14; SEQ ID NO:16; SEQ ID NO: 18; SEQ ID NO: 20; SEQ ID NO:22; and SEQ ID NO: 24.

5. The nucleic acid molecule of claim 1, wherein the nucleic acid molecule comprises one or more nucleotide sequences selected from the group consisting of:
m) SEQ ID NO:1, a coding sequence that is at least about 90% homologous to SEQ ID NO:1;
n) SEQ ID NO:3, a coding sequence that is at least about 90% homologous to SEQ ID NO:3;
o) SEQ ID NO:5, a coding sequence that is at least about 90% homologous to SEQ ID NO:5;
p) SEQ ID NO:7, a coding sequence that is at least about 90% homologous to SEQ ID NO:7;
q) SEQ ID NO:9, a coding sequence that is at least about 90% homologous to SEQ ID NO:9;
r) SEQ ID NO:11, a coding sequence that is at least about 90% homologous to SEQ ID NO:11;
s) SEQ ID NO:13, a coding sequence that is at least about 90% homologous to SEQ ID NO:13;
t) SEQ ID NO:15, a coding sequence that is at least about 90% homologous to SEQ ID NO:15;
u) SEQ ID NO:17, a coding sequence that is at least about 90% homologous to SEQ ID NO:17;
v) SEQ ID NO:19, a coding sequence that is at least about 90% homologous to SEQ ID NO:19;
w) SEQ ID NO:21, a coding sequence that is at least about 90% homologous to SEQ ID NO:21; and
x) SEQ ID NO:23, a coding sequence that is at least about 90% homologous to SEQ ID NO:23.

6. The nucleic acid molecule of claim 5, wherein the nucleic acid molecule comprises one or more nucleotide sequences selected from the group consisting of:
i) at least one selected from either elements a) or b);
ii) at least one selected from either elements c) or d);
iii) at least one selected from either elements e) or f);
iv) at least one selected from either elements g) or h);
v) at least one selected from either elements i) or j); and
vi) at least one selected from either elements k) or l).

7. The nucleic acid molecule of any one of claims 5-6, wherein the nucleic acid molecule comprises one or more nucleotide sequences selected from the group consisting of: SEQ ID NO:1; SEQ ID NO:3; SEQ ID NO:5; SEQ ID NO:7; SEQ ID NO:9; SEQ ID NO:11; SEQ ID NO:13; SEQ ID NO: 15; SEQ ID NO: 17; SEQ ID NO: 19; SEQ ID NO:21; and SEQ ID NO:23.

8. A nucleic acid molecule comprising one or more nucleotide sequences selected from the group consisting of:
a) SEQ ID NO:1, a coding sequence that is at least about 90% homologous to SEQ ID NO:1;
b) SEQ ID NO:3, a coding sequence that is at least about 90% homologous to SEQ ID NO:3;
c) SEQ ID NO:5, a coding sequence that is at least about 90% homologous to SEQ ID NO:5;
d) SEQ ID NO:7, a coding sequence that is at least about 90% homologous to SEQ ID NO:7;
e) SEQ ID NO:9, a coding sequence that is at least about 90% homologous to SEQ ID NO:9;
f) SEQ ID NO:11, a coding sequence that is at least about 90% homologous to SEQ ID NO:11;
g) SEQ ID NO:13, a coding sequence that is at least about 90% homologous to SEQ ID NO:13;
h) SEQ ID NO:15, a coding sequence that is at least about 90% homologous to SEQ ID NO:15;
i) SEQ ID NO:17, a coding sequence that is at least about 90% homologous to SEQ ID NO:17;
j) SEQ ID NO:19, a coding sequence that is at least about 90% homologous to SEQ ID NO:19;
k) SEQ ID NO:21, a coding sequence that is at least about 90% homologous to SEQ ID NO:21; and
l) SEQ ID NO:23, a coding sequence that is at least about 90% homologous to SEQ ID NO:23.

9. The nucleic acid molecule of claim 8, wherein the nucleic acid molecule comprises one or more nucleotide sequences selected from the group consisting of:
i) at least one selected from either elements a) or b);
ii) at least one selected from either elements c) or d);
iii) at least one selected from either elements e) or f);
iv) at least one selected from either elements g) or h);
v) at least one selected from either elements i) or j); and
vi) at least one selected from either elements k) or l).

10. The nucleic acid molecule of any one of claims 8-9, wherein the nucleic acid molecule comprises one or more nucleotide sequences selected from the group consisting of: SEQ ID NO:1; SEQ ID NO:3; SEQ ID NO:5; SEQ ID NO:7; SEQ ID NO:9; SEQ ID NO:11; SEQ ID NO:13; SEQ ID NO: 15; SEQ ID NO: 17; SEQ ID NO: 19; SEQ ID NO:21; and SEQ ID NO:23.

11. The nucleic acid molecule of claim 8, wherein the nucleic acid molecule encodes one or more proteins selected from the group consisting of:
y) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof,
z) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof,
aa) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof,
bb) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof,
cc) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof,
dd) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof,
ee) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof,
ff) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof,
gg) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof,
hh) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof,
ii) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and
jj) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

12. The nucleic acid molecule of any one of claims 1-11, wherein the nucleic acid molecule is a plasmid.

13. The nucleic acid molecule of any one of claims 1-12, wherein the nucleic acid molecule is an expression vector and sequences encoding said one or more proteins are operable linked to regulatory elements.

14. A composition comprising at least one nucleic acid molecule of any one of claims 1-13.

15. A method of treating a subject who has been diagnosed with prostate cancer comprising administering a nucleic acid molecule of any one of claims 1-13 to the subject.

16. A method of treating a subject who has been diagnosed with prostate cancer comprising administering a composition of claim 14 to the subject.

17. A method of inducing an immune response in a subject, the method comprising administering a nucleic acid molecule of any one of claims 1-13 to the subject.

18. A method of inducing an immune response in a subject, the method comprising administering a composition of claim 14 to the subject.

19. A protein comprising one or more proteins selected from the group consisting of:
kk) SEQ ID NO:2, a protein that is at least about 90% homologous to SEQ ID NO:2, an immunogenic fragment of SEQ ID NO:2, an immunogenic fragment of a protein that is at least about 90% homologous to SEQ ID NO:2, or any combination thereof,
ll) SEQ ID NO:4, a protein that is at least about 90% homologous to SEQ ID NO:4, an immunogenic fragment of SEQ ID NO:4, an immunogenic fragment of a protein that is at least about 90% homologous to SEQ ID NO:4, or any combination thereof,
mm) SEQ ID NO:6, a protein that is at least about 90% homologous to SEQ ID NO:6, an immunogenic fragment of SEQ ID NO:6, an immunogenic fragment of a protein that is at least about 90% homologous to SEQ ID NO:6, or any combination thereof,
nn) SEQ ID NO:8, a protein that is at least about 90% homologous to SEQ ID NO:8, an immunogenic fragment of SEQ ID NO:8, an immunogenic fragment of a protein that is at least about 90% homologous to SEQ ID NO:8, or any combination thereof,
oo) SEQ ID NO:10, a protein that is at least about 90% homologous to SEQ ID NO:10, an immunogenic fragment of SEQ ID NO:10, an immunogenic fragment of a protein that is at least about 90% homologous to SEQ ID NO:10, or any combination thereof,
pp) SEQ ID NO:12, a protein that is at least about 90% homologous to SEQ ID NO:12, an immunogenic fragment of SEQ ID NO:12, an immunogenic fragment of a protein that is at least about 90% homologous to SEQ ID NO:12, or any combination thereof,
qq) SEQ ID NO:14, a protein that is at least about 90% homologous to SEQ ID NO:14, an immunogenic fragment of SEQ ID NO:14, an immunogenic fragment of a protein that is at least about 90% homologous to SEQ ID NO:14, or any combination thereof,
rr) SEQ ID NO:16, a protein that is at least about 90% homologous to SEQ ID NO:16, an immunogenic fragment of SEQ ID NO:16, an immunogenic fragment of a protein that is at least about 90% homologous to SEQ ID NO:16, or any combination thereof,
ss) SEQ ID NO:18, a protein that is at least about 90% homologous to SEQ ID NO:18, an immunogenic fragment of SEQ ID NO:18, an immunogenic fragment of a protein that is at least about 90% homologous to SEQ ID NO:18, or any combination thereof,
tt) SEQ ID NO:20, a protein that is at least about 90% homologous to SEQ ID NO:20, an immunogenic fragment of SEQ ID NO:20, an immunogenic fragment of a protein that is at least about 90% homologous to SEQ ID NO:20, or any combination thereof,
uu) SEQ ID NO:22, a protein that is at least about 90% homologous to SEQ ID NO:22, an immunogenic fragment of SEQ ID NO:22, an immunogenic fragment of a protein that is at least about 90% homologous to SEQ ID NO:22, or any combination thereof, and
vv) SEQ ID NO:24, a protein that is at least about 90% homologous to SEQ ID NO:24, an immunogenic fragment of SEQ ID NO:24, an immunogenic fragment of a protein that is at least about 90% homologous to SEQ ID NO:24, or any combination thereof.

20. The protein of claim 19, wherein the protein is selected from the group consisting of a), b), c), d), e), f), g), h), i), j), k), and l).

21. The protein of claim 19, wherein the protein comprises one or more proteins selected from the group consisting of:
i) at least one selected from either elements a) or b);
ii) at least one selected from either elements c) or d);
iii) at least one selected from either elements e) or f);
iv) at least one selected from either elements g) or h);
v) at least one selected from either elements i) or j); and
vi) at least one selected from either elements k) or l).

22. The protein of any one of claims 19-21, wherein the protein comprises one or more proteins selected from the group consisting of: SEQ ID NO:2; SEQ ID NO:4; SEQ ID NO:6; SEQ ID NO:8; SEQ ID NO:10; SEQ ID NO:12; SEQ ID NO: 14; SEQ ID NO: 16; SEQ ID NO: 18; SEQ ID NO: 20; SEQ ID NO: 22; and SEQ ID NO:24.

23. A composition comprising at least one protein of any one of claims 19-22.

24. A method of treating a subject who has been diagnosed with prostate cancer comprising administering a protein of any one of claims 19-22 to the subject.

25. A method of treating a subject who has been diagnosed with prostate cancer comprising administering a composition of claim 23 to the subject.

26. A method of inducing an immune response in a subject, the method comprising administering a protein of any one of claims 19-22 to the subject.

27. A method of inducing an immune response in a subject, the method comprising administering a composition of claim 23 to the subject.


SEQUENCE LISTING (26 sequences)

SEQ ID NO: 1 - DNA, 1164 bp, Artificial Sequence
  PAP Antigen Nucleic Acid Consensus Sequence
atgcgggctgctcctctgctgctggctcgggctgcttcactgtccctggggttcctgtttctgctgtttttctggctggataggtctgtgctggccaaggagctgaagttcgtgaccctggtgtttcgccacggcgacaggtccccaatcgataccttcccaacagaccccatcaaggagagctcctggccccagggctttggccagctgacacagctgggcatggagcagcactacgagctgggcgagtatatcaggaagagataccggaagttcctgaacgagagctataagcacgagcaggtgtacatccgctccaccgacgtggataggacactgatgtctgccatgaccaacctggccgccctgtttccccctgagggcgtgtctatctggaatcccatcctgctgtggcagcctatcccagtgcacacagtgcctctgagcgaggatcagctgctgtatctgccattcagaaactgcccccggtttcaggagctggagtccgagacactgaagtctgaggagttccagaagagactgcacccctacaaggactttatcgccacactgggcaagctgagcggcctgcacggacaggatctgttcggcatctggtccaaggtgtacgaccctctgtattgtgagtctgtgcacaacttcaccctgccaagctgggccacagaggataccatgacaaagctgagggagctgtccgagctgtctctgctgagcctgtatggcatccacaagcagaaggagaagtccagactgcagggcggcgtgctggtgaacgagatcctgaatcacatgaagcgggccacccagatcccttcttataagaagctgatcatgtacagcgcccacgataccacagtgtccggcctgcagatggccctggacgtgtataacggcctgctgccaccctacgcatcctgccacctgaccgagctgtatttcgagaagggcgagtactttgtggagatgtactatcgcaatgagacacagcacgagccctaccctctgatgctgccaggctgctctcctagctgtccactggagaggttcgcagagctggtgggacccgtgatccctcaggattggagcacagaatgtatgaccactaactcacaccaggggacagaagatagcactgattgataa

SEQ ID NO: 2 - protein, 386 aa, Artificial Sequence
  PAP Antigen Amino Acid Consensus Sequence
MRAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 3 - DNA, 1155 bp, Artificial Sequence
  PAP Antigen Nucleic Acid Consensus Sequence Lacking Start and Stop Codons
cgggctgctcctctgctgctggctcgggctgcttcactgtccctggggttcctgtttctgctgtttttctggctggataggtctgtgctggccaaggagctgaagttcgtgaccctggtgtttcgccacggcgacaggtccccaatcgataccttcccaacagaccccatcaaggagagctcctggccccagggctttggccagctgacacagctgggcatggagcagcactacgagctgggcgagtatatcaggaagagataccggaagttcctgaacgagagctataagcacgagcaggtgtacatccgctccaccgacgtggataggacactgatgtctgccatgaccaacctggccgccctgtttccccctgagggcgtgtctatctggaatcccatcctgctgtggcagcctatcccagtgcacacagtgcctctgagcgaggatcagctgctgtatctgccattcagaaactgcccccggtttcaggagctggagtccgagacactgaagtctgaggagttccagaagagactgcacccctacaaggactttatcgccacactgggcaagctgagcggcctgcacggacaggatctgttcggcatctggtccaaggtgtacgaccctctgtattgtgagtctgtgcacaacttcaccctgccaagctgggccacagaggataccatgacaaagctgagggagctgtccgagctgtctctgctgagcctgtatggcatccacaagcagaaggagaagtccagactgcagggcggcgtgctggtgaacgagatcctgaatcacatgaagcgggccacccagatcccttcttataagaagctgatcatgtacagcgcccacgataccacagtgtccggcctgcagatggccctggacgtgtataacggcctgctgccaccctacgcatcctgccacctgaccgagctgtatttcgagaagggcgagtactttgtggagatgtactatcgcaatgagacacagcacgagccctaccctctgatgctgccaggctgctctcctagctgtccactggagaggttcgcagagctggtgggacccgtgatccctcaggattggagcacagaatgtatgaccactaactcacaccaggggacagaagatagcactgat

SEQ ID NO: 4 - protein, 385 aa, Artificial Sequence
  PAP Antigen Amino Acid Consensus Sequence Lacking Start and Stop Codons
RAAPLLLARAASLSLGFLFLLFFWLDRSVLAKELKFVTLVFRHGDRSPIDTFPTDPIKESSWPQGFGQLTQLGMEQHYELGEYIRKRYRKFLNESYKHEQVYIRSTDVDRTLMSAMTNLAALFPPEGVSIWNPILLWQPIPVHTVPLSEDQLLYLPFRNCPRFQELESETLKSEEFQKRLHPYKDFIATLGKLSGLHGQDLFGIWSKVYDPLYCESVHNFTLPSWATEDTMTKLRELSELSLLSLYGIHKQKEKSRLQGGVLVNEILNHMKRATQIPSYKKLIMYSAHDTTVSGLQMALDVYNGLLPPYASCHLTELYFEKGEYFVEMYYRNETQHEPYPLMLPGCSPSCPLERFAELVGPVIPQDWSTECMTTNSHQGTEDSTD

SEQ ID NO: 5 - DNA, 936 bp, Artificial Sequence
  PARM1 Antigen Nucleic Acid Consensus Sequence
atggtgtataagaccctgtttgccctgtgcattctgaccgccggctggagagtccagtccctgccaacttccgcccctctgagcgtgtccctgcccaccaacatcgtgccccctaccacaatctggacaagctcccctcagaacaccgacgccgatacagcctccccatctaatggcacccacaacaatagcgtgctgccagtgaccgcaagcgccccaacatccctgctgcccaagaatatctctatcgagagccgggaggaggagatcacctccccaggctctaactgggagggcacaaataccgacccaagcccatccggcttctctagcacctccggcggcgtgcacctgaccacaaccctggaggagcactcctctggaacccctgaggcaggagtggcagccacactgtctcagagcgccgcagagccacccaccctgatcagcccccaggcccctgccagctccccatctagcctgtccacatctcctccagaggtgtttagcgcctccgtgacaaccaaccactcctctacagtgacctctacacagccaaccggagcaccaacagcaccagagagccctaccgaggagagctcctctgaccacacccctacatcccacgcaaccgcagagcctgtgccacaggagaagacaccccctacaaccgtgtctggcaaagtgatgtgcgagctgatcgatatggagacaaccacaaccttccctcgcgtgatcatgcaggaggtggagcacgcactgagctccggcagcatcgcagccatcaccgtgacagtgatcgccgtggtgctgctggtgtttggcgtggccgcctacctgaagatcaggcactctagctatggcagactgctggacgaccacgactacggctcttggggcaactataacaatcctctgtatgacgactcatgataa

SEQ ID NO: 6 - protein, 310 aa, Artificial Sequence
  PARM1 Antigen Amino Acid Consensus Sequence
MVYKTLFALCILTAGWRVQSLPTSAPLSVSLPTNIVPPTTIWTSSPQNTDADTASPSNGTHNNSVLPVTASAPTSLLPKNISIESREEEITSPGSNWEGTNTDPSPSGFSSTSGGVHLTTTLEEHSSGTPEAGVAATLSQSAAEPPTLISPQAPASSPSSLSTSPPEVFSASVTTNHSSTVTSTQPTGAPTAPESPTEESSSDHTPTSHATAEPVPQEKTPPTTVSGKVMCELIDMETTTTFPRVIMQEVEHALSSGSIAAITVTVIAVVLLVFGVAAYLKIRHSSYGRLLDDHDYGSWGNYNNPLYDDS

SEQ ID NO: 7 - DNA, 927 bp, Artificial Sequence
  PARM1 Antigen Nucleic Acid Consensus Sequence Lacking Start and Stop Codon
gtgtataagaccctgtttgccctgtgcattctgaccgccggctggagagtccagtccctgccaacttccgcccctctgagcgtgtccctgcccaccaacatcgtgccccctaccacaatctggacaagctcccctcagaacaccgacgccgatacagcctccccatctaatggcacccacaacaatagcgtgctgccagtgaccgcaagcgccccaacatccctgctgcccaagaatatctctatcgagagccgggaggaggagatcacctccccaggctctaactgggagggcacaaataccgacccaagcccatccggcttctctagcacctccggcggcgtgcacctgaccacaaccctggaggagcactcctctggaacccctgaggcaggagtggcagccacactgtctcagagcgccgcagagccacccaccctgatcagcccccaggcccctgccagctccccatctagcctgtccacatctcctccagaggtgtttagcgcctccgtgacaaccaaccactcctctacagtgacctctacacagccaaccggagcaccaacagcaccagagagccctaccgaggagagctcctctgaccacacccctacatcccacgcaaccgcagagcctgtgccacaggagaagacaccccctacaaccgtgtctggcaaagtgatgtgcgagctgatcgatatggagacaaccacaaccttccctcgcgtgatcatgcaggaggtggagcacgcactgagctccggcagcatcgcagccatcaccgtgacagtgatcgccgtggtgctgctggtgtttggcgtggccgcctacctgaagatcaggcactctagctatggcagactgctggacgaccacgactacggctcttggggcaactataacaatcctctgtatgacgactca

SEQ ID NO: 8 - protein, 309 aa, Artificial Sequence
  PARM1 Antigen Amino Acid Consensus Sequence Lacking Start and Stop Codon
VYKTLFALCILTAGWRVQSLPTSAPLSVSLPTNIVPPTTIWTSSPQNTDADTASPSNGTHNNSVLPVTASAPTSLLPKNISIESREEEITSPGSNWEGTNTDPSPSGFSSTSGGVHLTTTLEEHSSGTPEAGVAATLSQSAAEPPTLISPQAPASSPSSLSTSPPEVFSASVTTNHSSTVTSTQPTGAPTAPESPTEESSSDHTPTSHATAEPVPQEKTPPTTVSGKVMCELIDMETTTTFPRVIMQEVEHALSSGSIAAITVTVIAVVLLVFGVAAYLKIRHSSYGRLLDDHDYGSWGNYNNPLYDDS

SEQ ID NO: 9 - DNA, 954 bp, Artificial Sequence
  PCTA Antigen Nucleic Acid Consensus Sequence
atgctgtcactgaacaacctgcagaacatcatctataaccctgtcatccccttcgtcggaaccatcccagaccagctggaccccggcaccctgatcgtgatcaggggccacgtgccaagcgacgccgatagattccaggtggacctgcagaacggcagctccgtgaagcctcgggccgatgtggccttccactttaatccaaggtttaagagagccggctgcatcgtgtgcaacacactgatcaatgagaagtggggcagggaggagatcacctacgacacacccttcaagagagagaagtcctttgagatcgtgatcatggtgctgaaggataagttccaggtggccgtgaacggcaagcacaccctgctgtacggccacaggatcggccctgagaagatcgacacactgggcatctatggcaaggtcaatatccacagcatcggcttctccttttctagcgatctgcagtctacccaggcctcctctctggagctgacagagatcgtgagagagaacgtgcctaagagcggcaccccacagctgtccctgcccttcgccgcacggctgaataccccaatgggacctggaaggacagtggtggtgcagggagaggtgaacgccaatgccaagtcttttaacgtggacctgctggccggcaagagcaaggatatcgccctgcacctgaacccccggctgaatatcaaggccttcgtgcgcaactcctttctgcaggagtcttggggcgaggaggagcgcaatatcacatctttccccttcagccccggcatgtacttcgagatgatcatctattgcgacgtgcgggagtttaaggtggccgtgaatggcgtgcactccctggagtataagcaccgcttcaaggagctgagtagcattgacaccctggagattaacggagacattcacctgctggaagtgcggtcctggtgataa

SEQ ID NO: 10 - protein, 316 aa, Artificial Sequence [sequence omitted]

SEQ ID NO: 11 - DNA, 945 bp, Artificial Sequence
  PCTA Antigen Nucleic Acid Consensus Sequence Lacking Start and Stop Codon
ctgtcactgaacaacctgcagaacatcatctataaccctgtcatccccttcgtcggaaccatcccagaccagctggaccccggcaccctgatcgtgatcaggggccacgtgccaagcgacgccgatagattccaggtggacctgcagaacggcagctccgtgaagcctcgggccgatgtggccttccactttaatccaaggtttaagagagccggctgcatcgtgtgcaacacactgatcaatgagaagtggggcagggaggagatcacctacgacacacccttcaagagagagaagtcctttgagatcgtgatcatggtgctgaaggataagttccaggtggccgtgaacggcaagcacaccctgctgtacggccacaggatcggccctgagaagatcgacacactgggcatctatggcaaggtcaatatccacagcatcggcttctccttttctagcgatctgcagtctacccaggcctcctctctggagctgacagagatcgtgagagagaacgtgcctaagagcggcaccccacagctgtccctgcccttcgccgcacggctgaataccccaatgggacctggaaggacagtggtggtgcagggagaggtgaacgccaatgccaagtcttttaacgtggacctgctggccggcaagagcaaggatatcgccctgcacctgaacccccggctgaatatcaaggccttcgtgcgcaactcctttctgcaggagtcttggggcgaggaggagcgcaatatcacatctttccccttcagccccggcatgtacttcgagatgatcatctattgcgacgtgcgggagtttaaggtggccgtgaatggcgtgcactccctggagtataagcaccgcttcaaggagctgagtagcattgacaccctggagattaacggagacattcacctgctggaagtgcggtcctgg

SEQ ID NO: 12 - protein, 315 aa, Artificial Sequence
  PCTA Antigen Amino Acid Consensus Sequence Lacking Start and Stop Codon
LSLNNLQNIIYNPVIPFVGTIPDQLDPGTLIVIRGHVPSDADRFQVDLQNGSSVKPRADVAFHFNPRFKRAGCIVCNTLINEKWGREEITYDTPFKREKSFEIVIMVLKDKFQVAVNGKHTLLYGHRIGPEKIDTLGIYGKVNIHSIGFSFSSDLQSTQASSLELTEIVRENVPKSGTPQLSLPFAARLNTPMGPGRTVVVQGEVNANAKSFNVDLLAGKSKDIALHLNPRLNIKAFVRNSFLQESWGEEERNITSFPFSPGMYFEMIIYCDVREFKVAVNGVHSLEYKHRFKELSSIDTLEINGDIHLLEVRSW

SEQ ID NO: 13 - DNA, 375 bp, Artificial Sequence
  PSCA Antigen Nucleic Acid Consensus Sequence
atgaaagccgtcctgctggcactgctgatggccggcctggcactgcagcccggaaccgctctgctgtgctactcctgtaaagcccaggtgagcaacgaggactgcctgcaggtggagaattgtacccagctgggcgagcagtgctggacagccaggatcagagccgtgggcctgctgaccgtgatcagcaagggctgctccctgaactgcgtggacgattctcaggattactatgtgggcaagaagaacatcacctgctgtgacacagacctgtgcaatgccagcggcgcccacgccctgcagcccgccgccgcaatcctggctctgctgcccgccctgggactgctgctgtggggacctggacagctgtgataa

SEQ ID NO: 14 - protein, 123 aa, Artificial Sequence
  PSCA Antigen Amino Acid Consensus Sequence
MKAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 15 - DNA, 366 bp, Artificial Sequence
  PSCA Antigen Nucleic Acid Consensus Sequence Lacking Start and Stop Codon
aaagccgtcctgctggcactgctgatggccggcctggcactgcagcccggaaccgctctgctgtgctactcctgtaaagcccaggtgagcaacgaggactgcctgcaggtggagaattgtacccagctgggcgagcagtgctggacagccaggatcagagccgtgggcctgctgaccgtgatcagcaagggctgctccctgaactgcgtggacgattctcaggattactatgtgggcaagaagaacatcacctgctgtgacacagacctgtgcaatgccagcggcgcccacgccctgcagcccgccgccgcaatcctggctctgctgcccgccctgggactgctgctgtggggacctggacagctg

SEQ ID NO: 16 - protein, 122 aa, Artificial Sequence
  PSCA Antigen Amino Acid Consensus Sequence Lacking Start and Stop Codon
KAVLLALLMAGLALQPGTALLCYSCKAQVSNEDCLQVENCTQLGEQCWTARIRAVGLLTVISKGCSLNCVDDSQDYYVGKKNITCCDTDLCNASGAHALQPAAAILALLPALGLLLWGPGQL

SEQ ID NO: 17 - DNA, 348 bp, Artificial Sequence
  PSP94 Antigen Nucleic Acid Consensus Sequence
atgaacgtgctgctgggctccgtggtcattttcgctacattcgtcactctgtgcaacgcttcctgttactttattccaaacgagggggtgcccggcgactctaccaggaagtgcatggatctgaagggcaacaagcaccctatcaatagcgagtggcagaccgacaactgtgagacatgcacatgttacgagacagagatcagctgctgtaccctggtgtccacacccgtgggctatgacaaggataattgccagagaatcttcaagaaggaggattgtaagtacatcgtggtggagaaaaaggaccccaaaaagacctgttcagtcagcgaatggattatttgataa

SEQ ID NO: 18 - protein, 114 aa, Artificial Sequence
  PSP94 Antigen Amino Acid Consensus Sequence
MNVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 19 - DNA, 339 bp, Artificial Sequence
  PSP94 Antigen Nucleic Acid Consensus Sequence Lacking Start and Stop Codon
aacgtgctgctgggctccgtggtcattttcgctacattcgtcactctgtgcaacgcttcctgttactttattccaaacgagggggtgcccggcgactctaccaggaagtgcatggatctgaagggcaacaagcaccctatcaatagcgagtggcagaccgacaactgtgagacatgcacatgttacgagacagagatcagctgctgtaccctggtgtccacacccgtgggctatgacaaggataattgccagagaatcttcaagaaggaggattgtaagtacatcgtggtggagaaaaaggaccccaaaaagacctgttcagtcagcgaatggattatt

SEQ ID NO: 20 - protein, 113 aa, Artificial Sequence
  PSP94 Antigen Amino Acid Consensus Sequence Lacking Start and Stop Codon
NVLLGSVVIFATFVTLCNASCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 21 - DNA, 1023 bp, Artificial Sequence
  STEAP1 Antigen Nucleic Acid Consensus Sequence
atggaatcaaggaaggacatcaccaatcaggaggaactgtggaaaatgaagccccgaagaaatctggaagaggacgactacctgcataaggacaccggcgagacatccatgctgaagaggccagtgctgctgcacctgcaccagaccgcacacgccgacgagtttgattgcccctctgagctgcagcacacacaggagctgttcccacagtggcacctgcccatcaagatcgccgccatcatcgccagcctgacctttctgtacacactgctgagagaagtgatccaccccctggccacctcccaccagcagtacttctataagatccctatcctggtcatcaacaaggtgctgccaatggtgtccatcacactgctggccctggtgtacctgcctggcgtgatcgccgccatcgtgcagctgcacaatggcaccaagtataagaagtttccacactggctggataagtggatgctgacaaggaagcagttcggcctgctgtctttctttttcgccgtgctgcacgccatctacagcctgtcctatcccatgaggagaagctaccggtataagctgctgaactgggcctaccagcaggtgcagcagaataaggaggacgcctggatcgagcacgacgtgtggcgcatggagatctacgtgagcctgggaatcgtgggcctggcaatcctggccctgctggcagtgacctctatcccttctgtgagcgactccctgacatggcgggagtttcactacatccagtctaagctgggcatcgtgagcctgctgctgggcaccatccacgccctgatctttgcctggaacaagtggatcgatatcaagcagttcgtgtggtatacccctcccaccttcatgatcgccgtgttcctgcccatcgtggtgctgatctttaagagcatcctgttcctgccttgtctgcgcaagaagatcctgaagattcggcacgggtgggaagatgtgactaagatcaataagacagagatttgtagccagctgtgataa

SEQ ID NO: 22 - protein, 329 aa, Artificial Sequence
  STEAP1 Antigen Amino Acid Consensus Sequence
MESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTK

SEQ ID NO: 23 - DNA, 1014 bp, Artificial Sequence
  STEAP1 Antigen Nucleic Acid Consensus Sequence Lacking Start and Stop Codon
gaatcaaggaaggacatcaccaatcaggaggaactgtggaaaatgaagccccgaagaaatctggaagaggacgactacctgcataaggacaccggcgagacatccatgctgaagaggccagtgctgctgcacctgcaccagaccgcacacgccgacgagtttgattgcccctctgagctgcagcacacacaggagctgttcccacagtggcacctgcccatcaagatcgccgccatcatcgccagcctgacctttctgtacacactgctgagagaagtgatccaccccctggccacctcccaccagcagtacttctataagatccctatcctggtcatcaacaaggtgctgccaatggtgtccatcacactgctggccctggtgtacctgcctggcgtgatcgccgccatcgtgcagctgcacaatggcaccaagtataagaagtttccacactggctggataagtggatgctgacaaggaagcagttcggcctgctgtctttctttttcgccgtgctgcacgccatctacagcctgtcctatcccatgaggagaagctaccggtataagctgctgaactgggcctaccagcaggtgcagcagaataaggaggacgcctggatcgagcacgacgtgtggcgcatggagatctacgtgagcctgggaatcgtgggcctggcaatcctggccctgctggcagtgacctctatcccttctgtgagcgactccctgacatggcgggagtttcactacatccagtctaagctgggcatcgtgagcctgctgctgggcaccatccacgccctgatctttgcctggaacaagtggatcgatatcaagcagttcgtgtggtatacccctcccaccttcatgatcgccgtgttcctgcccatcgtggtgctgatctttaagagcatcctgttcctgccttgtctgcgcaagaagatcctgaagattcggcacgggtgggaagatgtgactaagatcaataagacagagatttgtagccagctg

SEQ ID NO: 24 - protein, 338 aa, Artificial Sequence
  STEAP1 Antigen Amino Acid Consensus Sequence Lacking Start and Stop Codon
ESRKDITNQEELWKMKPRRNLEEDDYLHKDTGETSMLKRPVLLHLHQTAHADEFDCPSELQHTQELFPQWHLPIKIAAIIASLTFLYTLLREVIHPLATSHQQYFYKIPILVINKVLPMVSITLLALVYLPGVIAAIVQLHNGTKYKKFPHWLDKWMLTRKQFGLLSFFFAVLHAIYSLSYPMRRSYRYKLLNWAYQQVQQNKEDAWIEHDVWRMEIYVSLGIVGLAILALLAVTSIPSVSDSLTWREFHYIQSKLGIVSLLLGTIHALIFAWNKWIDIKQFVWYTPPTFMIAVFLPIVVLIFKSILFLPCLRKKILKIRHGWEDVTKINKTEICSQL

SEQ ID NO: 25 - DNA, 54 bp, Artificial Sequence
  IgE Leader Sequence - Nucleic Acid
atggactggacatggattctgtttctggtcgctgccgcaacccgcgtgcattca

SEQ ID NO: 26 - protein, 18 aa, Artificial Sequence
  IgE Leader Sequence - Amino Acid
MDWTWILFLVAAATRVHS